(19) United States
(12) Patent Application Publication
(10) Pub. No.: US 2024/0209336 A1
(43) Pub. Date: Jun. 27, 2024

(54) GENETICALLY MODIFIED HUMAN CELL LINES AND USES THEREOF

(71) Applicant: SIGILON THERAPEUTICS, INC., Cambridge, MA (US)

(72) Inventors: Brian Richard Fluharty, Boston, MA (US); Elina Makino, Winchester, MA (US)

(21) Appl. No.: 18/285,102

(22) PCT Filed: Mar. 31, 2022
(86) PCT No.: PCT/US2022/022854

§ 371 Date: Sep. 29, 2023

Related U.S. Application Data
(60) Provisional application No. 63/168,727, filed on Mar. 31, 2021.

(51) Int. Cl. C12N 9/40; A61K 9/00; A61K 9/48; A61K 35/30; A61K 38/47; A61K 47/36; C12N 5/079

(52) U.S. Cl. CPC C12N 9/2465; A61K 9/0024; A61K 9/4816; A61K 35/30; A61K 38/47; A61K 47/36; C12N 5/0621; C12Y 302/01022; C12N 2510/00

(57) ABSTRACT

Described herein are genetically modified cells derived from a human cell and which contain at least one exogenous transcription unit inserted into one or more specific open chromatin regions (OCRs), as well as compositions, pharmaceutical preparations, and implantable devices comprising the genetically modified cells, and methods of using the same for preventing or treating a disease, disorder, or condition.

### CLAIM OF PRIORITY

This application claims priority to U.S. Provisional Application No. 63/168,727, filed Mar. 31, 2021. The entire disclosure of the foregoing application is incorporated by reference in its entirety.

### SEQUENCE LISTING

The instant application contains a Sequence Listing which has been submitted electronically in ASCII format and is hereby incorporated by reference in its entirety. Said ASCII copy, created on Mar. 30, 2022, is named S2225-7043WO_SL.txt and is 83,464 bytes in size.

### BACKGROUND

Treating chronic and genetic diseases by implanting cells engineered to produce a therapeutic substance capable of treating such diseases has exciting potential to improve the health of patients with such diseases. To fully achieve the potential of such therapies, the implanted cells must be capable of producing therapeutic levels of the desired therapeutic substance for several weeks, months or even longer under conditions in which a selection marker is undesirable. Thus, a general approach to achieve stable, high level expression of the therapeutic substance is to implant engineered cells from a monoclonal cell line in which the therapeutic substance is encoded by an exogenous coding sequence inserted into one or more locations in the cell genome. However, generating a suitable monoclonal cell line is a time-consuming and expensive research endeavor because it is unpredictable which genomic locations will allow long-term acceptable expression levels of the therapeutic substance without transgene silencing and with minimal negative effects on the functioning and viability of the engineered cells.

### SUMMARY

The present disclosure is based on the identification of specific open chromatin regions in human retinal pigment epithelial (RPE) cell lines that are suitable genomic insertion sites for an exogenous transcription unit, e.g., to achieve stable, high expression of a polypeptide encoded by the exogenous transcription unit.

Described herein is a genetically modified cell derived from a human cell, e.g., an immortalized human cell, and comprising at least one exogenous transcription unit inserted into at least one specific open chromatin region (OCR) located in chromosome 1, 2, 3, 4, 5, 6, 7, 8, 9, 10, 13 or 14. In an embodiment, the cell comprises insertions in a first set of OCRs, located in Chr. 1, 3, 6, 7 and 9. In an embodiment, the cell comprises insertions in a second set of 19 OCRs located in Chr. 2, 3, 4, 5, 6, 7, 8, 9, 10, 13 and 14. In an embodiment, none of the OCRs in the first set are in the second set.

In an embodiment, the location of the OCRs in a genetically modified cell described herein is defined by reference to a nucleotide sequence present in a genomic reference sequence for the corresponding chromosome (Chr), e.g., the applicable Chr sequence from hg38 (as defined herein).

In an embodiment, an OCR suitable for expression of an inserted exogenous transcription unit(s) comprises a nucleotide sequence selected from one of the OCR reference sequences shown in FIG. 1 (SEQ ID NOS: 1-23) or a nucleotide sequence that is at least 90%, 95%, 98%, or 99% identical to the reference sequence.

In an embodiment, a genomic insertion site (GIS) for the exogenous transcription unit(s) is located anywhere between two nucleotide positions that correspond to the first and last nucleotides of an OCR reference sequence described herein, or a subsequence thereof. In an embodiment, the GIS locations are defined by reference to certain nucleotide positions in the corresponding hg38 sequence, e.g., as shown in Table 1 below.

TABLE 1
Genomic insertion sites in two exemplary
GLA-expressing clonal cell lines.

| Chr | FIG. | Insertion Site | Clone 1 | Clone 2 |
|---|---|---|---|---|
| 1 | 1A | 234,417,834-234,417,837 | x | |
| 2 | 1B | 38,319,172-38,319,175 | | x |
| 2 | 1C | 207,252,999-207,253,002 | | x |
| 3 | 1D | 11,428,957-11,428,960 | x | |
| 3 | 1E | 28,334,833-28,334,836 | | x |
| 3 | 1F | 147,827,505-147,827,508 | | x |
| 4 | 1G | 74,869,264-74,869,267 | | x |
| 4 | 1H | 135,474,683-135,474,686 | | x |
| 5 | 1I | 61,952,696-61,952,699 | | x |
| 5 | 1J | 104,635,306-104,635,309 | | x |
| 6 | 1K | 35,671,304-35,671,307 | | x |
| 6 | 1L | 38,755,617-38,755,620 | | x |
| 6 | 1M | 48,101,137-48,101,140 | | x |
| 6 | 1N | 112,086,894-112,086,897 | x | |
| 6 | 1O | 119,129,613-119,129,616 | | x |
| 7 | 1P | 114,988,879-114,988,882 | x | |
| 7 | 1Q | 135,676,629-135,676,632 | | x |
| 8 | 1R | 100,900,739-100,900,742 | | x |
| 9 | 1S | 4,120,956-4,120,959 | | x |
| 9 | 1T | 109,286,607-109,286,610 | x | |
| 10 | 1U | 17,905,886-17,905,889 | | x |
| 13 | 1V | 31,244,554-31,244,557 | | x |
| 14 | 1W | 57,286,845-57,286,848 | | X |

In an embodiment, the genetically modified cell is derived from a human epithelial cell. In an embodiment, the genetically modified cell is derived from an RPE cell, e.g., an immortalized human RPE cell. In an embodiment, the genetically modified cell is derived from a human cell line available from the American Type Culture Collection (Manassas, VA), e.g., the ARPE-19 (ATCC® CRL-2302™) cell line or the hTERT RPE-1 (ATCC® CRL-4000™) cell line.

In some embodiments, the exogenous transcription unit comprises a promoter sequence operably linked to a coding sequence for a polypeptide and a poly A signal sequence operably linked to the coding sequence. The promoter, poly A signal sequences are preferably selected to achieve high expression of the polypeptide in the parental human cell line. In an embodiment, the genetically modified cell is derived from the ARPE-19 cell line, the promoter consists essentially of, or consists of, SEQ ID NO:24 or SEQ ID NO:25, and the poly A signal sequence consists essentially of, or consists of, SEQ ID NO:26. The coding sequence is preferably codon-optimized for expression of the polypeptide in the parental cell line. In an embodiment, the polypeptide is constitutively expressed by the genetically modified cell when the cell is cultured in vitro. In an embodiment, the polypeptide is an enzyme, e.g., alpha-galactosidase A (GLA or GALA).

The present disclosure also provides a composition comprising a plurality of genetically modified cells described herein and a method of manufacturing the composition. In an embodiment, the composition comprises a cell culture media or a storage medium. In an embodiment, the composition comprises a polymer solution in which the cells are suspended, e.g., a polymer solution described herein, e.g., comprising alginate and a cell-binding substance. In an embodiment, the method of manufacturing the composition comprises culturing a plurality of a genetically modified cell described herein until a desired number of cultured cells has been produced and combining the desired number of cultured cells with a cell culture media, a storage medium or a polymer solution.

In yet another aspect, the present disclosure provides a device comprising at least one cell-containing compartment which comprises a genetically modified cell described herein or a plurality of such cells. In some embodiments, the device comprises a polymer composition encapsulating the genetically modified cell(s). In an embodiment, the encapsulating polymer composition comprises at least one cell binding-substance (CBS), e.g., a cell binding peptide, e.g., RGD (SEQ ID NO: 34) or RGDSP (SEQ ID NO: 35). In some embodiments, the device further comprises at least one means for mitigating the foreign body response (FBR), as defined herein, to the device when the device is placed inside a subject. In an embodiment, the means for mitigating the FBR comprises an afibrotic compound or afibrotic polymer, as defined herein, disposed on an exterior surface of the device and/or within a barrier compartment surrounding the cell-containing compartment. In an embodiment, the afibrotic compound is a compound of Formula (I):

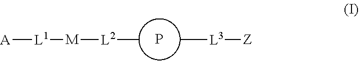

or a pharmaceutically acceptable salt thereof, wherein the variables A, Li, M, L², P, L³, and Z, as well as related subvariables, are defined herein. In some embodiments, the compound of Formula (I) or a pharmaceutically acceptable salt thereof (e.g., Formulas (I-a), (I-b), (I-b-i), (I-b-ii), (I-c), (I-d), (I-e), (I-f), (II), (II-a), (III), (III-a), (III-b), (III-c), (III-d), (III-e), (III-f), (III-g), (III-h), or (III-i), (III-e), (III-f), (III-g), (III-h), or (III-i) is a compound described herein, including for example, one of the compounds shown in Table 3 herein. In an embodiment, the afibrotic compound is Compound 100, Compound 101, or Compound 102 shown in Table 3.

In one aspect, a device of the disclosure is a 2-compartment hydrogel capsule (e.g., a microcapsule (less than 1 mm in diameter) or a millicapsule (at least 1 mm in diameter)) in which a cell-containing compartment (e.g., the inner compartment) comprising a plurality of live genetically modified cells described herein (and optionally one or more cell binding substances) is surrounded by a barrier compartment comprising an afibrotic polymer (e.g., the outer compartment, e.g., hydrogel layer). In an embodiment, the afibrotic polymer comprises an afibrotic compound. In an embodiment, the afibrotic compound is a compound of Formula (I).

In another aspect, the present disclosure features a preparation (e.g., a composition) comprising a plurality (at least any of 3, 6, 12, 25, 50 or more) of a cell-containing device described herein, e.g., a preparation of hydrogel capsules encapsulating genetically modified RPE cells. In some embodiments, the preparation is a pharmaceutically acceptable composition. In an embodiment, the hydrogel capsules in a hydrogel capsule preparation or composition comprise a population of hydrogel capsules that are sphere-like or spherical in shape, and optionally have a mean capsule diameter of about 500 μm to about 5000 μm (e.g., about 500 μm to about 4000 μm, about 500 μm to about 3000 μm, about 500 μm to about 2500 μm, about 500 μm to about 2000 μm, about 500 μm to about 1500 μm, about 500 μm to about 1000 μm, about 1000 μm to about 2500 μm). In an embodiment, the hydrogel capsules are not sphere-like or spherical in shape. In an embodiment, the hydrogel capsules in a hydrogel capsule preparation or composition comprise a population of hydrogel capsules that are sphere-like or spherical in shape, and optionally have a longest linear dimension (LLD) of about 500 μm to about 5000 μm (e.g., about 500 μm to about 4000 μm, about 500 μm to about 3000 μm, about 500 μm to about 2500 μm, about 500 μm to about 2000 μm, about 500 μm to about 1500 μm, about 500 μm to about 1000 μm, about 1000 μm to about 2500 μm). In an embodiment, the hydrogel capsules are not sphere-like or spherical in shape.

In another aspect, the present disclosure features a method of making or manufacturing a device comprising a genetically modified cell described herein. In some embodiments, the method comprises providing the genetically modified cell, or a plurality of such cells, and disposing the cell(s) in an enclosing component, e.g., a cell-containing compartment of the device as described herein. In some embodiments, the enclosing component comprises a flexible polymer (e.g., PLA, PLG, PEG, CMC, or a polysaccharide, e.g., alginate). In some embodiments, the enclosing component comprises an inflexible polymer or metal housing. In some embodiments, the surface of the device is chemically modified, e.g., with a compound of Formula (I) as described herein.

In an embodiment, a device described herein, or a plurality of devices, is combined with a pharmaceutically acceptable excipient to prepare a device preparation or a device composition which may be administered to a subject (e.g., into the intraperitoneal cavity) in need of treatment thereof with the therapeutic substance (e.g., exogenous polypeptide) produced by the device. In an embodiment, the genetically modified cells are derived from a human cell (e.g., an RPE cell, an ARPE-19 cell) and the device preparation or composition is capable of continuously delivering an effective amount of the therapeutic substance to the subject for a sustained time period, e.g., at least any of 3 months, 6 months, one year, two years or longer.

In another aspect, the present disclosure features a method of evaluating a composition, device or device preparation described herein. In some embodiments, the method comprises providing the composition, device or device preparation and evaluating a functional parameter of the composition, device or device preparation. In an embodiment, the functional parameter is the amount of the exogenous polypeptide produced by the cells in the composition, device or device preparation in vitro (e.g., when placed in a suitable culture medium) and/or in vivo (e.g., after implant into a subject, e.g., a non-human subject or a human subject.

In another aspect, the present disclosure features a method of treating a subject in need of therapy with an exogenous polypeptide expressed by a genetically modified cell described herein. The method comprises administering to the subject a device or device preparation comprising the genetically modified cell. In some embodiments, the administering step comprises placing into the subject a pharmaceutically acceptable preparation comprising a plurality of devices, each of which has the ability to produce the exogenous polypeptide. In some embodiments, the device or device preparation is administered to, placed in, or provided to a site other than the central nervous system, brain, spinal column, eye, or retina. In some embodiments, the implantable element is administered to, placed in, or injected in the peritoneal cavity (e.g., the lesser sac), the omentum, or the subcutaneous fat of a subject. In an embodiment, the method further comprises measuring the amount or activity of the exogenous polypeptide present in a tissue sample removed from the subject, e.g., in plasma separated from a blood sample, e.g., in a liver biopsy. In an embodiment, the tissue sample is removed at 15, 30, 60 or 120 days after administration, implantation, or placement of the device or device preparation. In some embodiments, the subject is a human. In an embodiment, the patient has been diagnosed with Fabry disease and the polypeptide is a GLA protein. In another embodiment, the patient has been diagnosed with Mucopolysaccharidosis type I (IPS I) and the polypeptide is an alpha-L-iduronidase protein.

The details of one or more embodiments of the disclosure are set forth herein. Other features, objects, and advantages of the disclosure will be apparent from the Detailed Description, the Figures, the Examples, and the Claims.

### BRIEF DESCRIPTION OF THE DRAWINGS

FIGS. 1A-1W show nucleotide sequences from Genome Reference Consortium Human Reference 38, Patch 12, Primary Assembly (hg38) for 23 OCRs that contain suitable genomic insertion sites for generating genetically modified cells of the present disclosure, with bold underlining indicating the 5′ and 3′ boundaries of specific insertion sites identified in two clonal cell lines, which were derived by transfecting ARPE-19 cells with a vector comprising an exogenous transcription unit encoding a GLA protein.

FIGS. 2A-2D show nucleotide sequences for various exogenous transcription unit elements that are useful to generate exemplary genetically modified cells of the present disclosure, with FIG. 2A and FIG. 2B showing nucleotide sequences of two different promoters (SEQ ID NO: 24 and SEQ ID NO:25), respectively, FIG. 2C showing the nucleotide sequence of a poly A signal sequence (SEQ ID NO:26), and FIG. 2D showing the nucleotide sequence of a complete exemplary transcription unit (SEQ ID NO:27), with underlining indicating the promoter sequence, shading indicating the Kozak sequence, [ORF] indicating the location of a coding sequence for a polypeptide of interest, and bold italics indicating the polyA signal sequence.

FIG. 3A shows the amino acid sequence (SEQ ID NO:28) of an exemplary human precursor GLA protein, with underlining indicating the signal peptide.

FIGS. 3B and 3C show exemplary codon optimized nucleotide sequences (SEQ ID NO:29 and SEQ ID NO:30) for expressing the human GLA protein of FIG. 3A in a genetically modified cell of the present disclosure.

FIG. 3D shows the amino acid sequence (SEQ ID NO:31) of an exemplary GLA fusion protein, with underlining indicating the heterologous signal peptide.

FIG. 3E shows an exemplary codon optimized nucleotide sequence (SEQ ID NO:32) for expressing the GLA fusion protein of FIG. 3D in a genetically modified cell of the present disclosure, with underlining indicating the coding sequence for the signal peptide.

FIG. 3F shows the nucleotide sequence (SEQ ID NO:33) of an exemplary transcription unit for expressing the GLA fusion protein of FIG. 3D in a genetically modified cell of the present disclosure, with underlining indicating the promoter sequence, bold, underlining indicating the coding sequence for the GLA fusion protein, and bold italics indicating the polyA signal sequence.

FIG. 4 illustrates an exemplary device of the disclosure (e.g., a two compartment hydrogel capsule), with lines indicating: a first, inner compartment formed from a polymer covalently attached to a cell binding peptide and genetically modified cells encapsulated therein; a second compartment (e.g., layer); and an afibrotic compound disposed within the second compartment and on the surface of the capsule. FIG. 4 discloses “GRGDSP” as SEQ ID NO: 37.

FIG. 5 compares the amount of GLA secreted into cell culture media from two clonal cell lines comprising multiple insertions of the exogenous transcription unit of FIG. 3F, as further described in the Examples herein.

### DETAILED DESCRIPTION

The present disclosure features genetically modified human cells (e.g., derived from human RPE cells, e.g., ARPE-19 cells), which have been engineered to produce an exogenous polypeptide of interest by inserting an exogenous transcription unit into one or more specific genomic OCRs that have been shown to allow stable, high level expression of a polypeptide encoded by the transcription unit. The present disclosure also provides compositions, devices and device preparations comprising such genetically modified cells. In some embodiments, the devices are configured to mitigate the FBR when placed inside a subject, e.g., a human subject. In some embodiments, the genetically modified cells, compositions, and devices are useful for delivering a therapeutic polypeptide to a subject in need of treatment with the therapeutic polypeptide. In some embodiments, the devices are configured to mitigate the FBR when placed inside a subject, e.g., a human subject.

### Definitions

So that the disclosure may be more readily understood, certain technical and scientific terms used herein are specifically defined below. Unless specifically defined elsewhere in this document, all other technical and scientific terms used herein have the meaning commonly understood by one of ordinary skill in the art to which this disclosure belongs.

As used herein, including the appended claims, the singular forms of words such as “a,” “an,” and “the,” include their corresponding plural references unless the context clearly dictates otherwise.

“About” or “approximately” when used herein to modify a numerically defined parameter (e.g., amount of genetically modified cells in a composition or device (e.g., hydrogel capsule), a physical description of a device such as diameter, sphericity, number of cells encapsulated therein, the number of devices in a preparation), means that the recited numerical value is within an acceptable functional range for the defined parameter as determined by one of ordinary skill in the art, which will depend in part on how the numerical value is measured or determined, e.g., the limitations of the measurement system, including the acceptable error range for that measurement system. For example, “about” can mean a range of 20% above and below the recited numerical value. As a non-limiting example, a device defined as having a diameter of about 1.5 millimeters (mm) and encapsulating about 5 million (M) cells may have a diameter of 1.2 to 1.8 mm and may encapsulate 4 M to 6 M cells. As another non-limiting example, a preparation of about 100 devices (e.g., hydrogel capsules) includes preparations having 80 to 120 devices. In some embodiments, the term “about” means that the modified parameter may vary by as much as 15%, 10% or 5% above and below the stated numerical value for that parameter.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., using a fluorescence microscope to acquire fluorescence microscopy data.

“Administer,” “administering,” or “administration,” as used herein, refer to implanting, absorbing, ingesting, injecting, placing or otherwise introducing into a subject, an entity described herein (e.g., a device or a preparation of devices), or providing such an entity to a subject for administration.

“Afibrotic”, as used herein, means a compound or material that mitigates the foreign body response (FBR) to an implanted device. For example, the amount of FBR in a biological tissue that is induced by implant into that tissue of a device (e.g., a hydrogel capsule) comprising an afibrotic compound (e.g., a hydrogel capsule comprising a polymer covalently modified with a compound listed in Table 3) is lower than the FBR induced by implantation of an afibrotic-null reference device, i.e., a device that lacks any afibrotic compound, but is of substantially the same composition (e.g., same CBP-polymer, same cell type(s)) and structure (e.g., size, shape, no. of compartments). In an embodiment, the degree of the FBR is assessed by the immunological response in the tissue containing the implanted device (e.g., hydrogel capsule), which may include, for example, protein adsorption, macrophages, multinucleated foreign body giant cells, fibroblasts, and angiogenesis, using assays known in the art, e.g., as described in WO 2017/075630, or using one or more of the assays/methods described Vegas, A., et al., Nature Biotechnol (supra), (e.g., subcutaneous cathepsin measurement of implanted capsules, Masson's trichrome (MT), hematoxylin or eosin staining of tissue sections, quantification of collagen density, cellular staining and confocal microscopy for macrophages (CD68 or F4/80), myofibroblasts (alpha-muscle actin, SMA) or general cellular deposition, quantification of 79 RNA sequences of known inflammation factors and immune cell markers, or FACS analysis for macrophage and neutrophil cells on retrieved devices (e.g., capsules) after 14 days in the intraperitoneal space of a suitable test subject, e.g., an immunocompetent mouse. In an embodiment, the FBR is assessed by measuring the levels in the tissue containing the implant of one or more biomarkers of immune response, e.g., cathepsin, TNF-α, IL-13, IL-6, G-CSF, GM-CSF, IL-4, CCL2, or CCL4. In some embodiments, the FBR induced by a device of the invention (e.g., a hydrogel capsule comprising an afibrotic compound disposed on its outer surface), is at least about 80%, about 85%, about 90%, about 95%, about 99%, or about 100% lower than the FBR induced by an FBR-null reference device, e.g., a device that is substantially identical to the test or claimed device except for lacking the means for mitigating the FBR (e.g., a hydrogel capsule that does not comprise an afibrotic compound but is otherwise substantially identical to the claimed capsule. In some embodiments, the FBR (e.g., level of a biomarker(s)) is measured after about 30 minutes, about 1 hour, about 6 hours, about 12 hours, about 1 day, about 2 days, about 3 days, about 4 days, about 1 week, about 2 weeks, about 1 month, about 2 months, about 3 months, about 6 months, or longer.

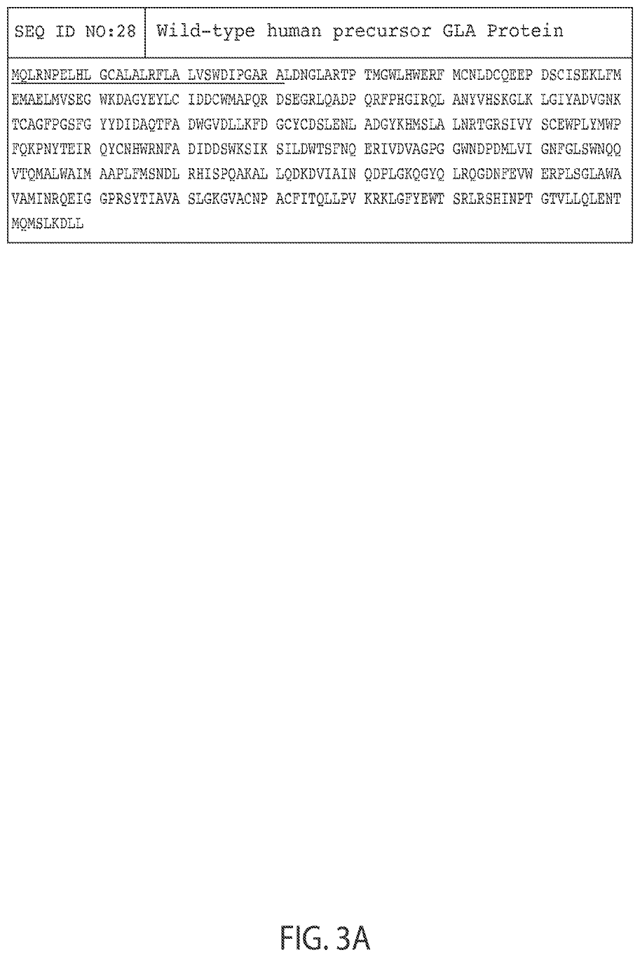

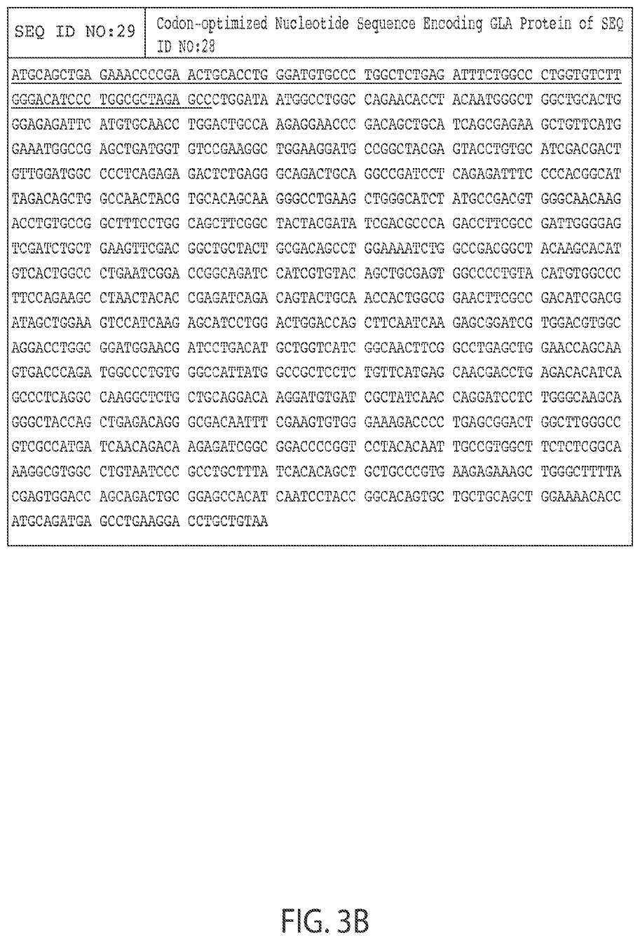

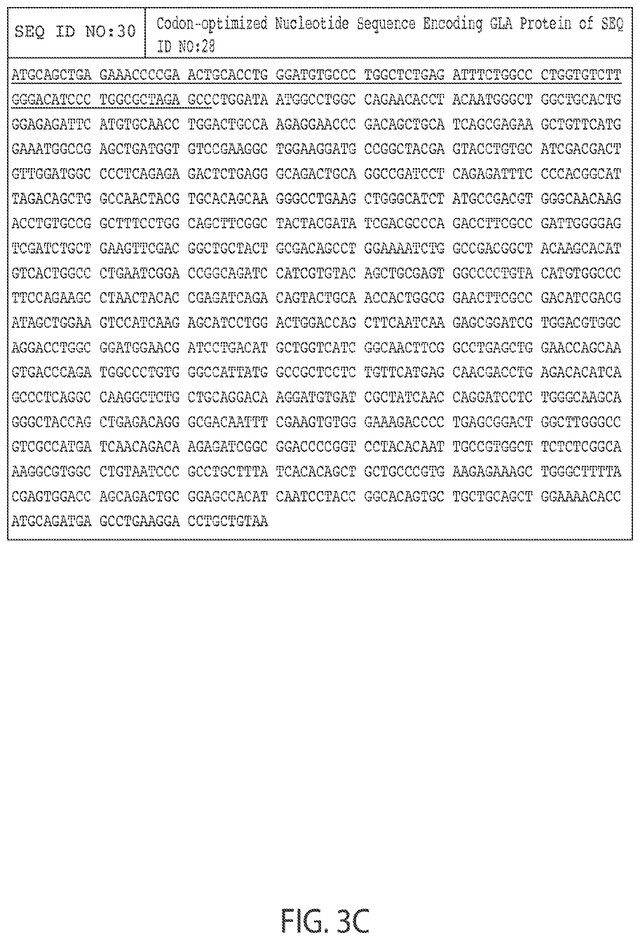

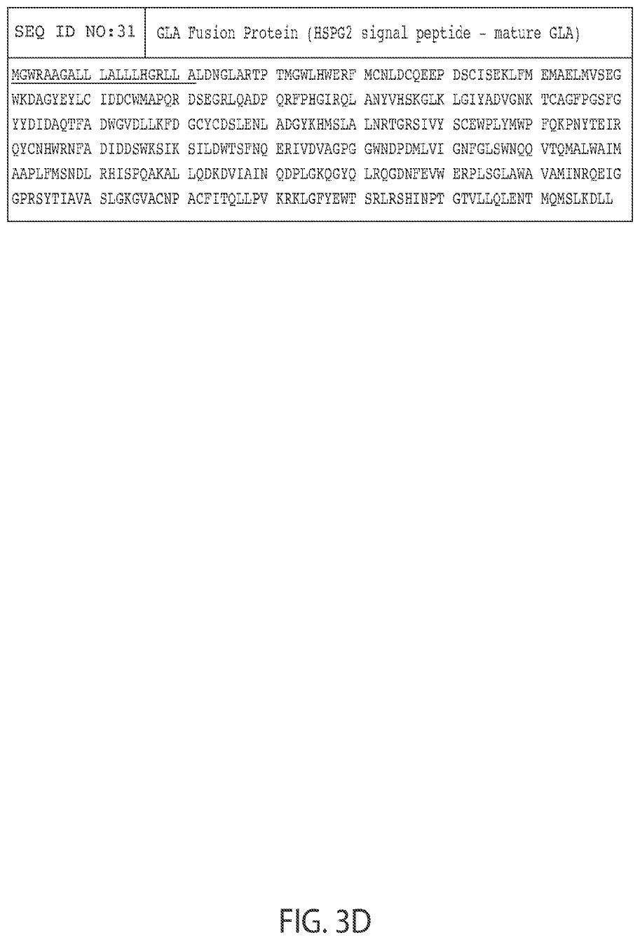

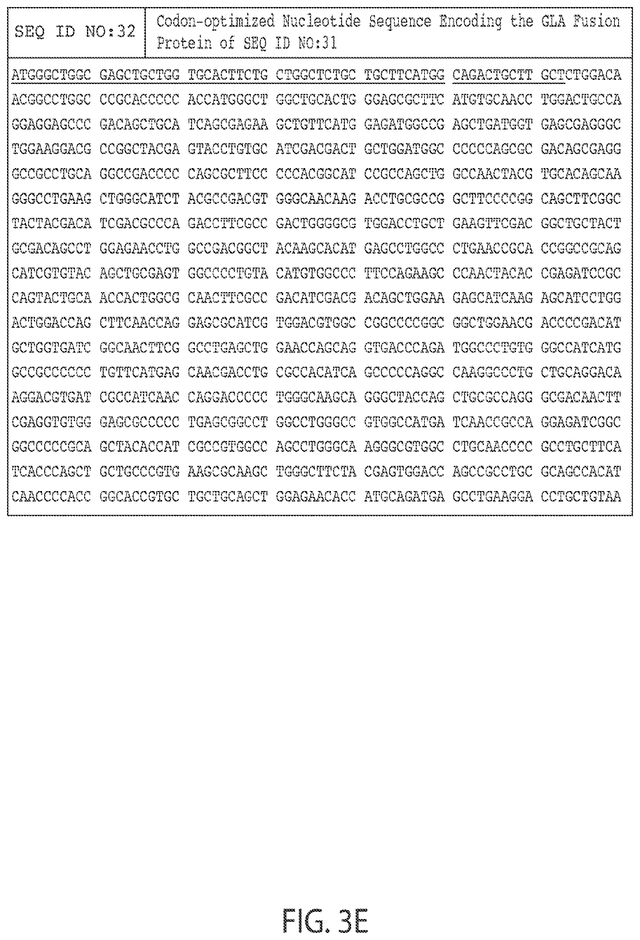

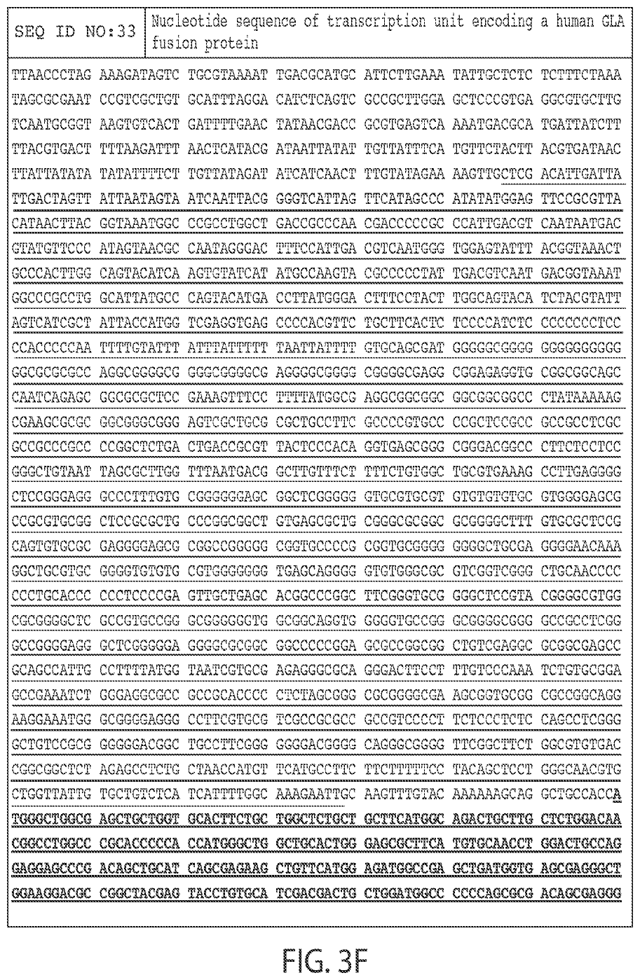

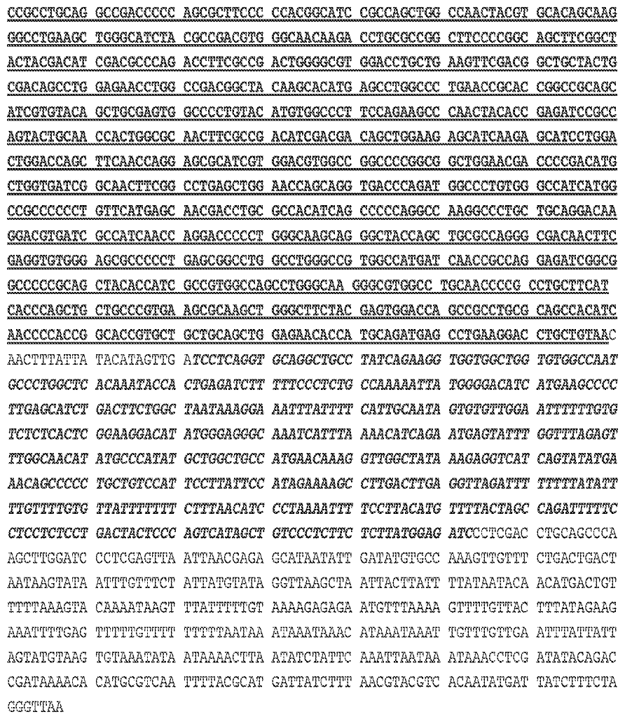

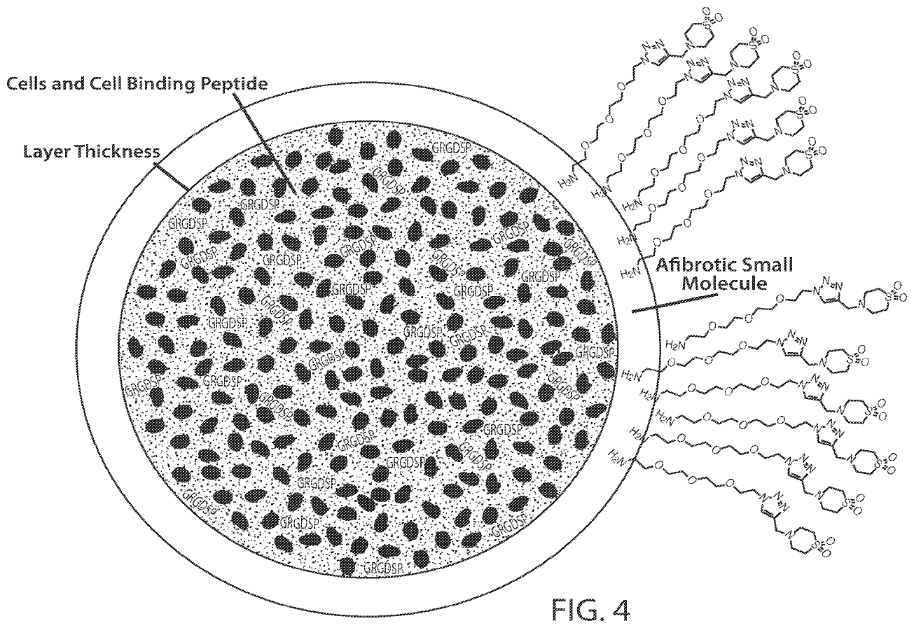

“Alpha-galactosidase A”, “α-Gal A”, “alpha-D-galactosidase-A”, “alpha-galactoside galactohydrolase”, “galactosidase alpha”, and “GLA protein” may be used interchangeably herein and refer to a homodimeric protein comprising the mature amino acid sequence encoded by a wild-type mammalian GLA gene or an amino acid sequence with conservative substitutions thereof. In an embodiment, the conservatively substituted GLA protein has enzyme activity that is within 80-120%, 85-115%, 90-110% or 95-105% of the corresponding wild-type mammalian mature GLA protein, as measured by an art recognized GLA activity assay. The wild-type human GLA gene encodes a 429-amino acid polypeptide, of which the N-terminal 31 amino acids constitute a signal peptide. The amino acid sequence for wild-type human precursor α-Gal A is shown in FIG. 3A (SEQ ID NO:28). In some embodiments, the term “GLA protein” (and any of the aforesaid synonyms) refers to a polypeptide comprising the wild-type mature amino acid sequence shown in FIG. 3A, and optionally preceded by the GLA signal peptide (underlined in FIG. 3A) or by a signal peptide for a different secretory protein, e.g., a protein secreted by human cells (e.g., human epithelial cells), e.g., the signal peptide for HSPG2, as shown in FIG. 3D, which is also referred to herein as a GLA fusion protein.

GLA activity can be directly measured in blood leukocytes from a subject, lysing of the cells, and determining the enzymatic activity in the lysate upon addition of an enzyme substrate such as 4-methylumbelliferal alpha-D-galactoside. Immunoassays for measuring GLA activity and protein to determine the concentrations of alpha-galactosidase in blood and plasma are described in Fuller et al., Clin Chem. 2004; 50(11):1979-85. In an embodiment, GLA activity is measured in culture media or in a urine sample or tissue sample collected from a subject (e.g., plasma separated from blood, a homogenate of a liver, kidney, or heart tissue sample) using the enzymatic assay described in the Examples below.

Indirect assessments of GLA activity are based on measuring a substrate e.g., levels of Gb3 and biomarker lysoGb3 in blood plasma and/or urine sample collected from the subject or in a biopsy of a tissue of interest, e.g., liver, kidney, heart. Gb3 and lysoGb3 levels can be measured using the assay described in the Examples herein or any assay known in the art. For example, a method for measuring Gb3 levels in plasma and urine of humans affected by Fabry disease is described in, e.g., Boscaro et al., Rapid Commun Mass Spectrom. 2002; 16(16):1507-14. In this method, the analyses are performed using flow injection analysis-electrospray ionization-tandem mass spectrometry (FIA-ESI-MS/MS). Gb3 accumulation in skin biopsies obtained using a “punch” device may be detected using an immunoelectron-microscopic method such as described in Kanekura et al., Br J Dermatol. 2005, 153(3):544-8. Various biopsy techniques and assays for detecting Gb3 and other surrogate biomarkers are described in US patent application publication US 2010/0113517. Other plasma surrogate biomarkers of GLA activity and/or Fabry disease progression (e.g., various inflammatory and cardiac remodeling biomarkers) are described in Yogasundaram, H. et al., J Am Heart Assoc. 2018; 7:e009098.

“Alpha-L-iduronidase protein” or “IDUA protein” as used herein means a polypeptide that comprises the mature amino acid sequence encoded by a wild-type mammalian IDUA gene or an amino acid sequence with conservative substitutions thereof, which is capable of hydrolyzing nonreducing terminal alpha-L-iduronic acid residues in glycosaminoglycans (GAGs) (e.g., dermatan sulfate and heparan sulfate). In an embodiment, the conservatively substituted IDUA protein has enzyme activity that is within 80-120%, 85-115%, 90-110% or 95-105% of the corresponding wild-type mammalian mature IDUA protein, as measured by an art recognized IDUA enzyme activity assay, e.g., hydrolysis of the substrate 4-methylumbelliferyl-α-L-iduronide (4MU-iduronide), see, e.g., Ou, L. et al., Mol Genet Metab. 2014 February: 111(2): 113-115. IDUA protein that may be expressed by a genetically modified cell described herein (e.g., derived from a human epithelial cell line, e.g., the ARPE-19 cell line), include wild-type primate (e.g., human), porcine, canine, and murine proteins, as well as variants of such wild-type proteins, including fragments, mutants, variants (including silent variants) with one or more amino acid substitutions and/or deletions. The wild-type human IDUA gene encodes a 653 amino acid precursor protein, of which the N-terminal 26 amino acids constitute a signal peptide.

“Cell,” as used herein, refers to a human cell that is engineered (genetically modified) or a human cell that is not engineered (not genetically modified). In an embodiment, a cell is an immortalized cell, or an engineered cell derived from an immortalized human cell line. In an embodiment, the cell is a live cell, e.g., is viable as measured by any technique described herein or known in the art.

“Cell-binding peptide (CBP)”, as used herein, means a linear or cyclic peptide that comprises an amino acid sequence that is derived from the cell binding domain of a ligand for a cell-adhesion molecule (CAM) (e.g., that mediates cell-matrix junctions or cell-cell junctions). The CBP is less than 50, 40 30, 25, 20, 15 or 10 amino acids in length. In an embodiment, the CBP is between 3 and 12 amino acids, 4 and 10 amino acids in length, or is 3, 4, 5, 6, 7 8, 9 or 10 amino acids in length. The CBP amino acid sequence may be identical to the naturally occurring binding domain sequence or may be a conservatively substituted variant thereof. In an embodiment, the CAM ligand is a mammalian protein. In an embodiment, the CAM ligand is a human protein selected from the group of proteins listed in Table 1 in published international application WO 2020/198685. In an embodiment, the CBP comprises, consists essentially of, or consists of a cell binding sequence listed in Table 1 of WO 2020/198685 or a conservatively substituted variant thereof. In an embodiment, the CBP is an RGD peptide (SEQ ID NO: 34), which means the peptide comprises the amino acid sequence RGD (SEQ ID NO:34) and optionally comprises one or more additional amino acids located at the N-terminus and/or the C-terminus. In an embodiment, the CBP is a cyclic peptide comprising RGD (SEQ ID NO: 34), e.g., one of the cyclic RGD peptides (SEQ ID NO: 34) described in Vilaca, H. et al., Tetrahedron 70 (35):5420-5427 (2014). In an embodiment, the CBP is a linear peptide comprising RGD (SEQ ID NO: 34) and is less than 6 amino acids in length. In an embodiment, the CBP is a linear peptide that consists essentially of RGD (SEQ ID NO: 34) or RGDSP (SEQ ID NO:35).

“CBP-polymer”, as used herein, means a polymer comprising at least one cell-binding peptide molecule covalently attached to the polymer via a linker. In an embodiment, the polymer in the CBP-polymer is not a peptide or a polypeptide. In an embodiment, the polymer in a CBP-polymer is a synthetic or naturally occurring polysaccharide, e.g., an alginate, e.g., a sodium alginate. In an embodiment, the linker is an amino acid linker (i.e., consists essentially of a single amino acid, or a peptide of several identical or different amino acids), which is joined via a peptide bond to the N-terminus or C-terminus of the CBP. In an embodiment, the C-terminus of an amino acid linker is joined to the N-terminus of the CBP and the N-terminus of the amino acid linker is joined to at least one pendant carboxyl group in the polysaccharide via an amide bond. In an embodiment, the structure of the linker-CBP is expressed as G₍₁₋₄₎-CBP, meaning that the linker has one, two, three or four glycine residues (“G₍₁₋₄₎” disclosed as SEQ ID NO: 40). In an embodiment, one or more of the monosaccharide moieties in a CBP-polysaccharide, e.g., a CBP-alginate) is not modified with the CBP, e.g., the unmodified moiety has a free carboxyl group or lacks a modifiable pendant carboxyl group. In an embodiment, the number of polysaccharide moieties with a covalently attached CBP is less than any of the following values: 99%, 95%, 90%, 80%, 70%, 60%, 50%, 40% 30%, 20%, 10%, 5%, 1%.

“Cell-binding polypeptide (CBPP)”, as used herein, means a polypeptide of at least 50, at least 75, or at least 100 amino acids in length and comprising the amino acid sequence of a cell binding domain of a CAM ligand, or a conservatively substituted variant thereof. In an embodiment, the CAM ligand is a mammalian protein. In an embodiment, the CBPP amino acid comprises the naturally occurring amino acid sequence of a full-length CAM ligand, e.g., one of the proteins listed in Table 1 or WO 2020/198685, or a conservatively substituted variant thereof.

“Cell-binding substance (CBS)”, as used herein, means any chemical, biological or other type of substance (e.g., a small organic compound, a peptide, a polypeptide) that is capable of mimicking at least one activity of a ligand for a cell-adhesion molecule (CAM) or other cell-surface molecule that mediates cell-matrix junctions or cell-cell junctions or other receptor-mediated signaling. In an embodiment, when present in a polymer composition encapsulating live cells, the CBS is capable of forming a transient or permanent bond or contact with one or more of the cells. In an embodiment, the CBS facilitates interactions between two or more live cells encapsulated in the polymer composition. In an embodiment, the CBS is physically attached to one or more polymer molecules in the polymer composition. In an embodiment, the CBS is a cell-binding peptide or cell-binding polypeptide, as defined herein.

“Conservatively modified variants” or conservative substitution”, as used herein, refers to a variant of a reference peptide or polypeptide that is identical to the reference molecule, except for having one or more conservative amino acid substitutions in its amino acid sequence. In an embodiment, a conservatively modified variant consists of an amino acid sequence that is at least 70%, 80%, 85%, 90%, 95%, 97%, 98% or 99% identical to the reference amino acid sequence. A conservative amino acid substitution refers to substitution of an amino acid with an amino acid having similar characteristics (e.g., charge, side-chain size, hydrophobicity/hydrophilicity, backbone conformation and rigidity, etc.) and which has minimal impact on the biological activity of the resulting substituted peptide or polypeptide. Conservative substitution tables of functionally similar amino acids are well known in the art, and exemplary substitutions grouped by functional features are set forth in Table 2 below.

TABLE 2
Exemplary conservative amino acid substitution groups.

| Feature | Conservative Amino Group |
|---|---|
| Charge/Polarity | His, Arg, Lys |
| | Asp, Glu |
| | Cys, Thr, Ser, Gly, Asn, Gln, Tyr |
| | Ala, Pro, Met, Leu, Ile, Val, Phe, Trp |
| Hydrophobicity | Asp, Glu, Asn, Gln, Arg, Lys |
| | Cys, Ser, Thr, Pro, Gly, His, Tyr |
| | Ala, Met, Ile Leu, Val, Phe, Trp |
| Structural/Surface | Asp, Glu, Asn, Aln, His, Arg, Lys |
| Exposure | Cys, Ser, Tyr, Pro, Ala, Gly, Trp, Tyr |
| | Met, Ile, Leu, Val, Phe |
| Secondary Structure | Ala, Glu, Aln, His, Lys, Met, Leu, Arg |
| Propensity | Cys, Thr, Ile, Val, Phe, Tyr, Trp |
| | Ser, Gly, Pro, Asp, Asn |
| Evolutionary | Asp, Glu |
| Conservation | His, Lys, Arg |
| | Asn, Gln |
| | Ser, Thr |
| | Leu, Ile, Val |
| | Phe, Tyr, Trp |
| | Ala, Gly |
| | Met, Cys |

“Consists essentially of”, and variations such as “consist essentially of” or “consisting essentially of” as used throughout the specification and claims, indicate the inclusion of any recited elements or group of elements, and the optional inclusion of other elements, of similar or different nature than the recited elements, that do not materially change the basic or novel properties of the specified molecule, composition, device, or method. As a non-limiting example, a cell-binding peptide, a GLA protein or an FVIII protein that consists essentially of a recited amino acid sequence may also include one or more amino acids, including substitutions in the recited amino acid sequence, of one or more amino acid residues, which do not materially affect the relevant biological activity of the cell-binding peptide, the GLA protein or the FVIII protein, respectively.

“Derived from”, as used herein with respect to a cell or cells, refers to cells obtained from tissue, cell lines, or cells, which optionally are then cultured, passaged, immortalized, genetically modified, differentiated and/or induced, etc. to produce the derived cell(s).

“Device”, as used herein, refers to any implantable object (e.g., a particle, a hydrogel capsule, an implant, a medical device), which contains one or more live, genetically modified human cells (e.g., derived from RPE cells) capable of expressing, and optionally secreting, an exogenous polypeptide following implant of the device, and has a configuration that supports the viability of the cells by allowing cell nutrients to enter the device. In some embodiments, the device allows release from the device of metabolic byproducts generated by the cells.

“Effective amount”, as used herein, refers to an amount of genetically modified cells (e.g., derived from human cells (e.g., epithelial cells)) producing an exogenous polypeptide or a device preparation producing the polypeptide that is sufficient to elicit a desired biological response. In an embodiment, the desired biological response is an increase in levels of the exogenous polypeptide within the cells, or for secreted polypeptides, in a tissue sample removed from a subject treated with (e.g., implanted with) the genetically modified cells, a device or a device preparation containing such cells. As will be appreciated by those of ordinary skill in this art, the effective amount may vary depending on such factors as the desired biological endpoint, the pharmacokinetics of the exogenous polypeptide, composition or device, the condition being treated, the mode of administration, and the age and health of the subject. An effective amount encompasses therapeutic and prophylactic treatment.

An “endogenous nucleic acid” as used herein, is a nucleic acid that occurs naturally in a subject cell.

An “endogenous polypeptide,” as used herein, is a polypeptide that occurs naturally in a subject cell.

“Engineered human cell” and “genetically modified human cell”, may be used interchangeably herein, and each term means a human cell (e.g., an epithelial cell) having a non-naturally occurring genetic alteration (e.g., in the cellular genome), and typically comprises an exogenous nucleic acid sequence (e.g., DNA or RNA) not present (or present at a different level than) in an otherwise similar human cell (e.g., epithelial cell) that is not engineered. In an embodiment, an engineered human cell (e.g., engineered RPE cell) comprises an exogenous nucleic acid encoding a polypeptide, e.g., a therapeutic protein. In an embodiment, the exogenous nucleic acid sequence is chromosomal (e.g., the exogenous nucleic acid sequence is an exogenous sequence disposed in endogenous chromosomal sequence) or is extra chromosomal (e.g., a non-integrated expression vector). In an embodiment, the exogenous nucleic acid sequence comprises an RNA sequence, e.g., an mRNA. In an embodiment, the exogenous nucleic acid sequence comprises a chromosomal or extra-chromosomal exogenous nucleic acid sequence that comprises a sequence which is expressed as RNA, e.g., mRNA or a regulatory RNA. In an embodiment, the exogenous nucleic acid sequence comprises a first chromosomal or extra-chromosomal exogenous nucleic acid sequence that modulates the conformation or expression of a second nucleic acid sequence the second nucleic acid sequence can be exogenous or endogenous. For example, an engineered cell can comprise an exogenous nucleic acid that controls the expression of an endogenous sequence. In an embodiment, the engineered cell comprises an exogenous nucleic acid sequence which comprises a codon optimized coding sequence for a polypeptide of interest and achieves higher expression of the polypeptide than a naturally-occurring coding sequence. The codon optimized coding sequence may be generated using a commercially available algorithm, e.g., GeneOptimizer (ThermoFisher Scientific), OptimumGene™ (GenScript, Piscataway, NJ USA), GeneGPS® (ATUM, Newark, CA USA), or Java Codon Adaptation Tool (JCat, www.jcat.de, Grote, A. et al., Nucleic Acids Research, Vol 33, Issue suppl 2, pp. W526-W531 (2005). In an embodiment, an engineered cell (e.g., engineered epithelial cell, e.g., engineered RPE cell, e.g., engineered ARPE-19 cell) is cultured from a monoclonal cell line. In some embodiments, the engineered cell is not an islet cell, as defined herein.

An “exogenous nucleic acid,” as used herein, is a nucleic acid that does not occur naturally in a subject cell.

An “exogenous polypeptide,” as used herein, is a polypeptide that is encoded by an exogenous nucleic acid in a subject cell. Reference to an amino acid position of a specific sequence means the position of said amino acid in a reference amino acid sequence, e.g., sequence of a full-length mature (after signal peptide cleavage) wild-type protein (unless otherwise stated), and does not exclude the presence of variations, e.g., deletions, insertions and/or substitutions at other positions in the reference amino acid sequence.

“Factor VII protein” or “FVII protein” as used herein, means a polypeptide that comprises the amino acid sequence of a naturally occurring factor VII protein or variant thereof that has a FVII biological activity, e.g., promoting blood clotting, as determined by an art-recognized assay, unless otherwise specified. Naturally occurring FVII exists as a single chain zymogen, a zymogen-like two-chain polypeptide and a fully activated two-chain form (FVIIa). In some embodiments, reference to FVII includes single-chain and two-chain forms thereof, including zymogen-like and FVIIa. FVII proteins that may be produced by a genetically modified cell described herein (e.g., derived from a human epithelial cell line, e.g., the ARPE-19 cell line), include wild-type primate (e.g., human), porcine, canine, and murine proteins, as well as variants of such wild-type proteins, including fragments, mutants, variants with one or more amino acid substitutions and/or deletions. In some embodiments, a variant FVII protein is capable of being activated to the fully activated two-chain form (Factor VIIa) that has at least 50%, 75%, 90% or more (including >100%) of the activity of wild-type Factor VIIa. Variants of FVII and FVIIa are known, e.g., marzeptacog alfa (activated) (MarzAA) and the variants described in European Patent No. 1373493, U.S. Pat. Nos. 7,771,996, 9,476,037 and US published application No. US20080058255.

Factor VII biological activity may be quantified by an art recognized assay, unless otherwise specified. For example, FVII biological activity in a sample of a biological fluid, e.g., plasma, may be quantified by (i) measuring the amount of Factor Xa produced in a system comprising tissue factor (TF) embedded in a lipid membrane and Factor X (Persson et al., J. Biol. Chem. 272:19919-19924, 1997); (ii) measuring Factor X hydrolysis in an aqueous system; (iii) measuring its physical binding to TF using an instrument based on surface plasmon resonance (Persson, FEBS Letts. 413:359-363, 1997); or (iv) measuring hydrolysis of a synthetic substrate; and/or (v) measuring generation of thrombin in a TF-independent in vitro system. In an embodiment, FVII activity is assessed by a commercially available chromogenic assay (BIOPHEN FVII, HYPHEN BioMed Neuville sur Oise, France), in which the biological sample containing FVII is mixed with thromboplastin calcium, Factor X and SXa-11 (a chromogenic substrate specific for Factor Xa.

“Factor VIII protein” or “FVIII protein” as used herein, means a polypeptide that comprises the amino acid sequence of a naturally occurring factor VIII polypeptide or variant thereof that has an FVIII biological activity, e.g., coagulation activity, as determined by an art-recognized assay, unless otherwise specified. FVIII proteins that may be expressed by a genetically modified cell described herein (e.g., derived from a human epithelial cell line, e.g., the ARPE-19 cell line), include wild-type primate (e.g., human), porcine, canine, and murine proteins, as well as variants of such wild-type proteins, including fragments, mutants, variants with one or more amino acid substitutions and/or deletions, B-domain deletion (BDD) variants, single chain variants and fusions of any of the foregoing wild-type or variants with a half-life extending polypeptide. In an embodiment, the cells are engineered to encode a precursor factor VIII polypeptide (e.g., with the signal sequence) with a full or partial deletion of the B domain. In an embodiment, the cells are engineered to encode a single chain factor VIII polypeptide which contains a variant FVIII protein preferably has at least 50%, 75%, 90% or more (including >100%) of the coagulation activity of the corresponding wild-type factor VIII. Assays for measuring the coagulation activity of FVIII proteins include the one stage or two stage coagulation assay (Rizza et al., 1982, Coagulation assay of FVIII:C and FIXa in Bloom ed. The Hemophelias. NY Churchill Livingston 1992) or the chromogenic substrate FVIII:C assay (Rosen, S. 1984. Scand J Haematol 33:139-145, suppl.).

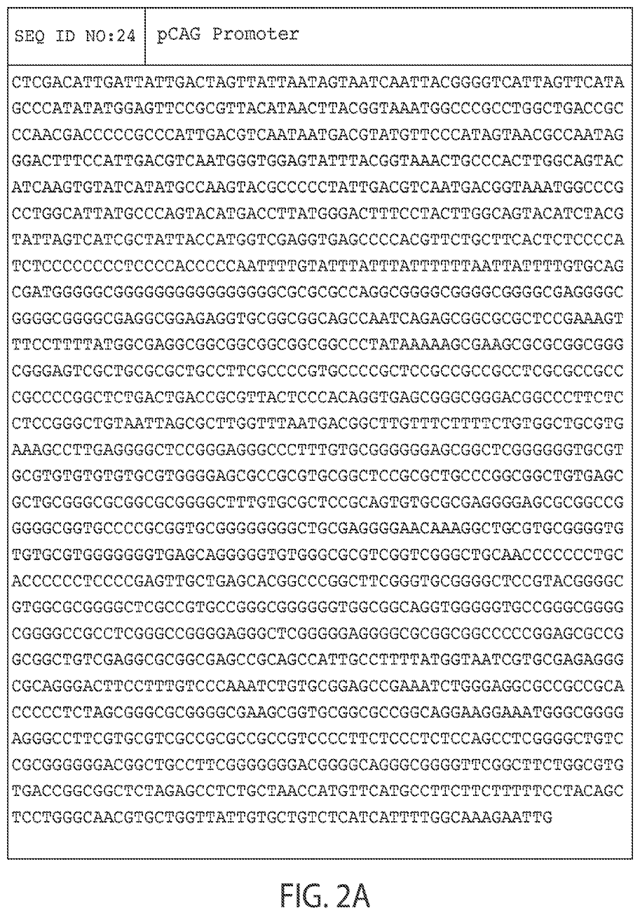

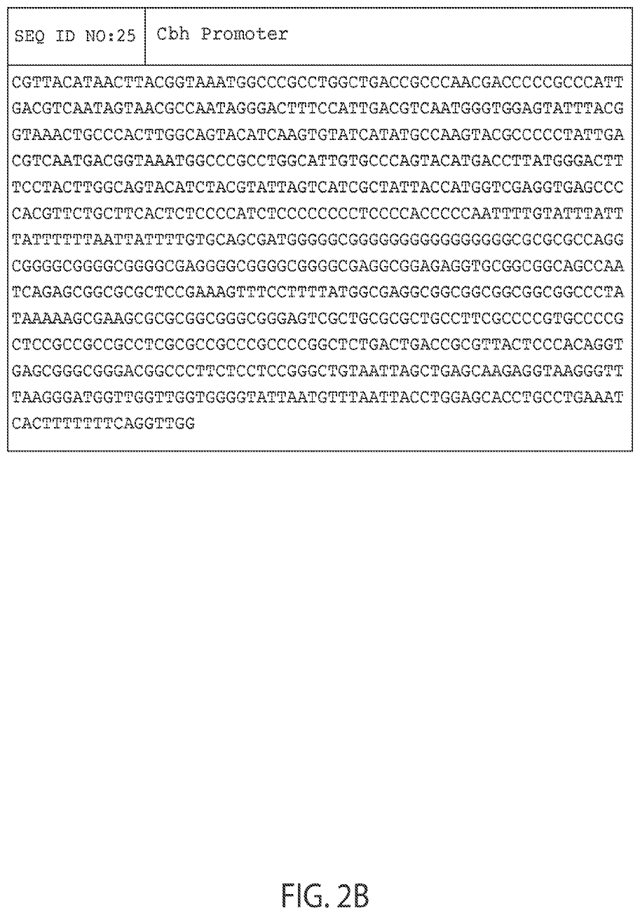

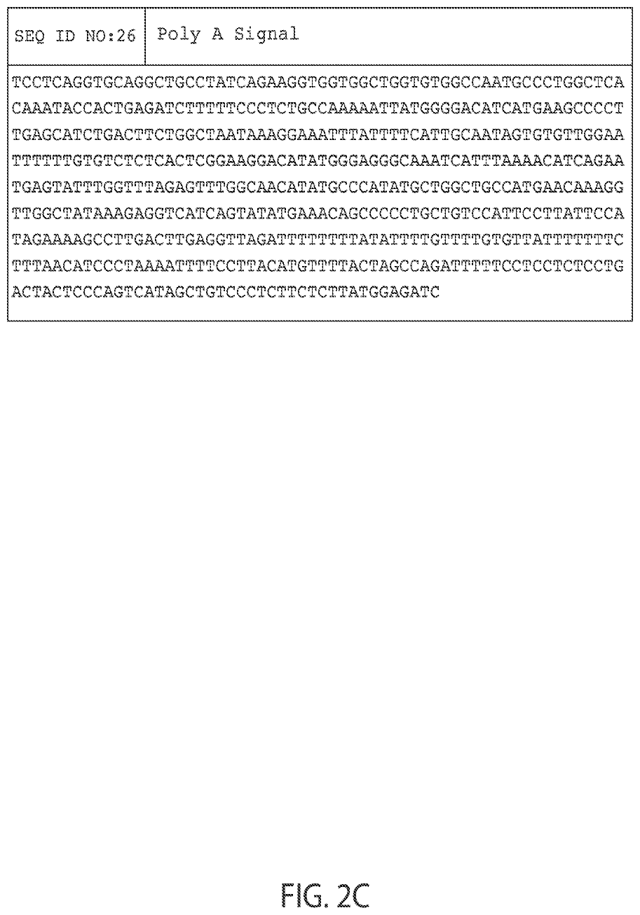

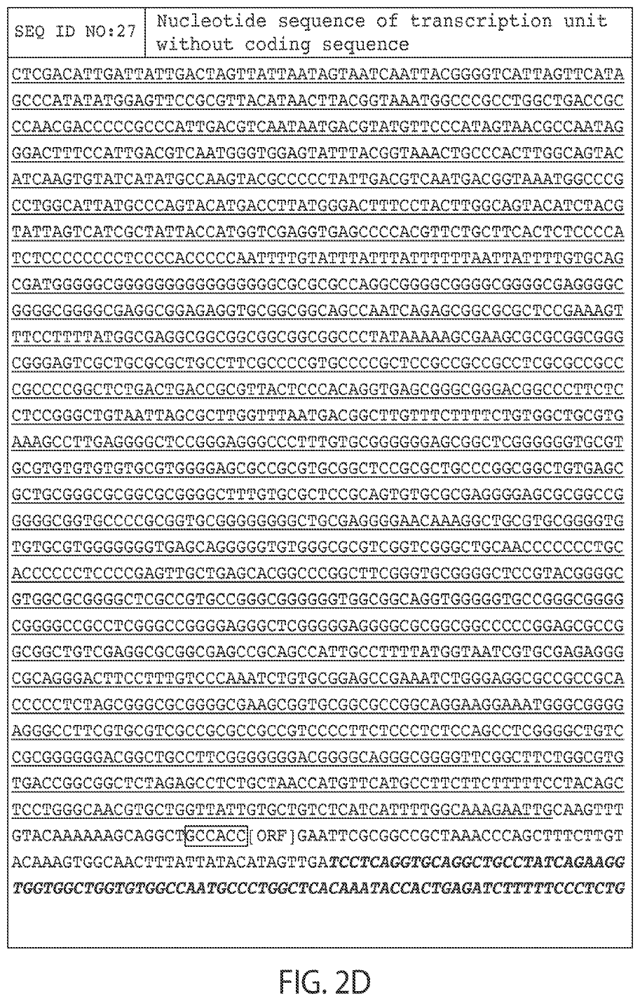

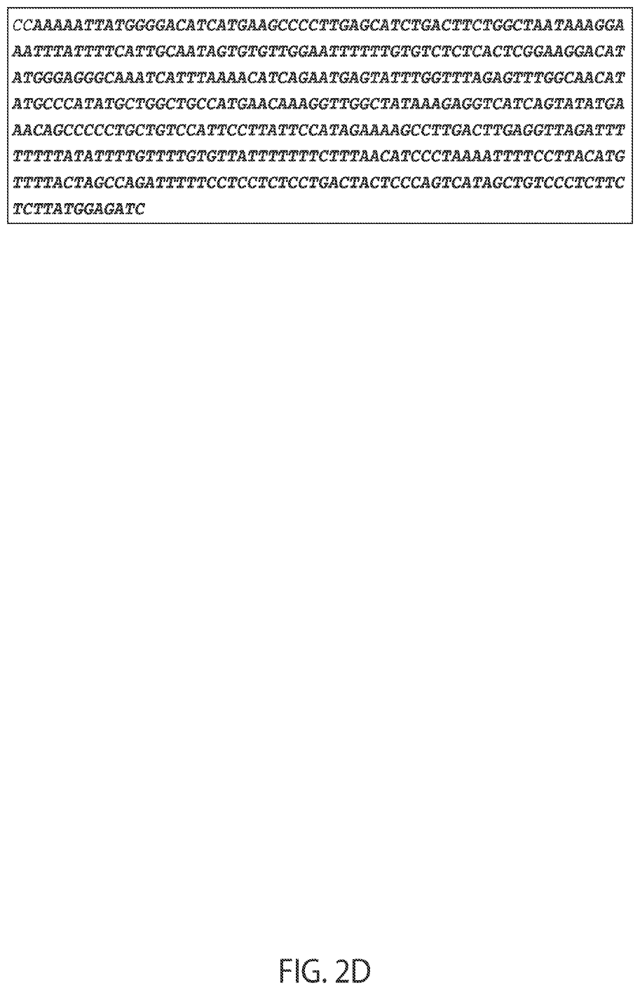

A number of FVIII-BDD variants are known, and include, e.g., variants with the full or partial B-domain deletions disclosed in any of the following U.S. Pat. No. 4,868,112 (e.g., col. 2, line 2 to col. 19, line 21 and table 2); U.S. Pat. No. 5,112,950 (e.g., col. 2, lines 55-68, FIG. 2, and example 1); U.S. Pat. No. 5,171,844 (e.g., col. 4, line 22 to col. 5, line 36); U.S. Pat. No. 5,543,502 (e.g., col. 2, lines 17-46); U.S. Pat. Nos. 5,595,886; 5,610,278; 5,789,203 (e.g., col. 2, lines 26-51 and examples 5-8); U.S. Pat. No. 5,972,885 (e.g., col. 1, lines 25 to col. 2, line 40); U.S. Pat. No. 6,048,720 (e.g., col. 6, lines 1-22 and example 1); U.S. Pat. Nos. 6,060,447; 6,228,620; 6,316,226 (e.g., col. 4, line 4 to col. 5, line 28 and examples 1-5); U.S. Pat. Nos. 6,346,513; 6,458,563 (e.g., col. 4, lines 25-53) and U.S. Pat. No. 7,041,635 (e.g., col. 2, line 1 to col. 3, line 19, col. 3, line 40 to col. 4, line 67, col. 7, line 43 to col. 8, line 26, and col. 11, line 5 to col. 13, line 39).

In some embodiments, a FVIII-BDD protein produced by a genetically modified cell described herein (e.g., derived from a human epithelial cell line, e.g., the ARPE-19 cell line) has one or more of the following deletions of amino acids in the B-domain: (i) most of the B domain except for amino-terminal B-domain sequences essential for intracellular processing of the primary translation product into two polypeptide chains (WO 91/09122); (ii) a deletion of amino acids 747-1638 (Hoeben R. C., et al. J. Biol. Chem. 265 (13): 7318-7323 (1990)); amino acids 771-1666 or amino acids 868-1562 (Meulien P., et al. Protein Eng. 2(4):301-6 (1988); amino acids 982-1562 or 760-1639 (Toole et al., Proc. Natl. Acad. Sci. U.S.A. 83:5939-5942 (1986)); amino acids 797-1562 (Eaton et al., Biochemistry 25:8343-8347 (1986)); 741-1646 (Kaufman, WO 87/04187)), 747-1560 (Sarver et al., DNA 6:553-564 (1987)); amino acids 741-1648 (Pasek, WO 88/00831)), amino acids 816-1598 or 741-1689 (Lagner (Behring Inst. Mitt. (1988) No 82:16-25, EP 295597); a deletion that includes one or more residues in a furin protease recognition sequence, including any of the specific deletions recited in U.S. Pat. No. 9,956,269 at col. 10, line 65 to col. 11, line 36.

In other embodiments, a FVIII-BDD protein retains any of the following B-domain amino acids or amino acid sequences: (i) one or more N-linked glycosylation sites in the B-domain, e.g., residues 757, 784, 828, 900, 963, or optionally 943, first 226 amino acids or first 163 amino acids (Miao, H. Z., et al., Blood 103(a): 3412-3419 (2004), Kasuda, A., et al., J. Thromb. Haemost. 6: 1352-1359 (2008), and Pipe, S. W., et al., J. Thromb. Haemost. 9: 2235-2242 (2011).

In some embodiments, the FVIII-BDD protein is a single-chain variant generated by substitution or deletion of one or more amino acids in the furin protease recognition sequence LKRHQR (SEQ ID NO: 36) that prevents proteolytic cleavage at this site, including any of the substitutions at the R1645 and/or R1648 positions described in U.S. Pat. Nos. 10,023,628, 9,394,353 and 9,670,267.

In some embodiments, any of the above FVIII-BDD proteins may further comprise one or more of the following variations: a F309S substitution to improve expression of the FVIII-BDD protein (Miao, H. Z., et al., Blood 103(a): 3412-3419 (2004); albumin fusions (WO 2011/020866); and Fc fusions (WO 04/101740).

All FVIII-BDD amino acid positions referenced herein refer to the positions in full-length human FVIII, unless otherwise specified.

“Factor IX protein” or “FIX protein”, as used herein, means a polypeptide that comprises the amino acid sequence of a naturally occurring factor IX protein or variant thereof that has a FIX biological activity, e.g., coagulation activity, as determined by an art-recognized assay, unless otherwise specified. FIX is produced as an inactive zymogen, which is converted to an active form by factor XIa excision of the activation peptide to produce a heavy chain and a light chain held together by one or more disulfide bonds. FIX proteins that may be produced by a genetically modified described herein (e.g., derived from an RPE cell line, e.g., the ARPE-19 cell line), include wild-type primate (e.g., human), porcine, canine, and murine proteins, as well as variants of such wild-type proteins, including fragments, mutants, variants with one or more amino acid substitutions and/or deletions and fusions of any of the foregoing wild-type or variant proteins with a half-life extending polypeptide. In an embodiment, cells are engineered to encode a full-length wild-type human factor IX polypeptide (e.g., with the signal sequence) or a functional variant thereof Δ variant FIX protein preferably has at least 50%, 75%, 90% or more (including >100%) of the coagulation activity of wild-type factor VIX. Assays for measuring the coagulation activity of FIX proteins include the Biophen Factor IX assay (Hyphen BioMed) and the one stage clotting assay (activated partial thromboplastin time (aPTT), e.g., as described in EP 2 032 607, thrombin generation time assay (TGA) and rotational thromboelastometry, e.g., as described in WO 2012/006624.

A number of functional FIX variants are known and may be expressed by engineered cells encapsulated in a device described herein, including any of the functional FIX variants described in the following international patent publications: WO 02/040544 at page 4, lines 9-30 and page 15, lines 6-31; WO 03/020764 in Tables 2 and 3 at pages 14-24, and at page 12, lines 1-27; WO 2007/149406 at page 4, line 1 to page 19, line 11; WO 2007/149406 A2 at page 19, line 12 to page 20, line 9; WO 08/118507 at page 5, line 14 to page 6, line 5; WO 09/051717 at page 9, line 11 to page 20, line 2; WO 09/137254 at page 2, paragraph [006] to page 5, paragraph [011] and page 16, paragraph [044] to page 24, paragraph [057]; WO 09/130198 A2 at page 4, line 26 to page 12, line 6; WO 09/140015 at page 11, paragraph [0043] to page 13, paragraph [0053]; WO 2012/006624; WO 2015/086406.

In certain embodiments, the FIX polypeptide comprises a wild-type or variant sequence fused to a heterologous polypeptide or non-polypeptide moiety extending the half-life of the FIX protein. Exemplary half-life extending moieties include Fc, albumin, a PAS sequence, transferrin, CTP (28 amino acid C-terminal peptide (CTP) of human chorionic gonadotropin (hCG) with its 4 O-glycans), polyethylene glycol (PEG), hydroxyethyl starch (HES), albumin binding polypeptide, albumin-binding small molecules, or any combination thereof Δn exemplary FIX polypeptide is the rFIXFc protein described in WO 2012/006624, which is an FIXFc single chain (FIXFc-sc) and an Fc single chain (Fc-sc) bound together through two disulfide bonds in the hinge region of Fc.

FIX variants also include gain and loss of function variants. An example of a gain of function variant is the “Padua” variant of human FIX, which has a L (leucine) at position 338 of the mature protein instead of an R (arginine) (corresponding to amino acid position 384 of SEQ ID NO:20), and has greater catalytic and coagulant activity compared to wild-type human FIX (Chang et al., J. Biol. Chem., 273:12089-94 (1998)). An example of a loss of function variant is an alanine substituted for lysine in the fifth amino acid position from the beginning of the mature protein, which results in a protein with reduced binding to collagen IV (e.g., loss of function).

“Islet cell”, as used herein, means a cell that comprises any naturally occurring or any synthetically created, or modified, cell that is intended to recapitulate, mimic or otherwise express, in part or in whole, the functions, in part or in whole, of the cells of the pancreatic islets of Langerhans. The term “islet cell” includes a glucose-responsive, insulin producing cell derived from a stem cell, e.g., from an induced pluripotent stem cell line.

“Polymer composition”, as used herein, is a composition (e.g., a solution, mixture) comprising one or more polymers. As a class, “polymers’ includes homopolymers, heteropolymers, co-polymers, block polymers, block co-polymers and can be both natural and synthetic. Homopolymers contain one type of building block, or monomer, whereas co-polymers contain more than one type of monomer.

“Polypeptide”, as used herein, refers to a polymer comprising amino acid residues linked through peptide bonds and having at least two, and in some embodiments, at least 3, 4, 5, 10, 50, 75, 100, 150 or 200 amino acid residues.

“Prevention,” “prevent,” and “preventing” as used herein refers to a treatment that comprises administering a composition (or preparation) of devices encapsulating genetically modified cells that express an exogenous polypeptide, prior to the onset of one or more symptoms of a disease or condition that is amenable to treatment with the exogenous polypeptide, to preclude the physical manifestation of the symptom(s). In some embodiments, “prevention,” “prevent,” and “preventing” require that signs or symptoms of a disease or condition have not yet developed or have not yet been observed.

“RPE cell” as used herein refers to a cell having one or more of the following characteristics: a) it comprises a retinal pigment epithelial cell (RPE) (e.g., cultured using an RPE cell line, e.g., the ARPE-19 cell line (ATCC® CRL-2302™)) or a cell derived or engineered therefrom, e.g., by stably transfecting cells cultured from the ARPE-19 cell line with an exogenous sequence that encodes a polypeptide of interest or inserting the exogenous sequence into one of the specific OCR insertion sites described herein, a cell derived from a primary cell culture of RPE cells, a cell isolated directly (without long term culturing, e.g., less than 5 or 10 passages or rounds of cell division since isolation) from naturally occurring RPE cells, e.g., from a human or other mammal, a cell derived from a transformed, an immortalized, or a long term (e.g., more than 5 or 10 passages or rounds of cell division) RPE cell culture; b) a cell that has been obtained from a less differentiated cell, e.g., a cell developed, programmed, or reprogramed (e.g., in vitro) into an RPE cell or a cell that is, except for any genetic engineering, substantially similar to one or more of a naturally occurring RPE cell or a cell from a primary or long term culture of RPE cells (e.g., the cell can be derived from an IPS cell); or c) a cell that has one or more of the following properties: i) it expresses one or more of the biomarkers CRALBP, RPE-65, RLBP, BEST1, or αB-crystallin; ii) it does not express one or more of the biomarkers CRALBP, RPE-65, RLBP, BEST1, or αB-crystallin; iii) it is naturally found in the retina and forms a monolayer above the choroidal blood vessels in the Bruch's membrane; or iv) it is responsible for epithelial transport, light absorption, secretion, and immune modulation in the retina; or v) it has been created synthetically, or modified from a naturally occurring cell, to have the same or substantially the same genetic content, and optionally the same or substantially the same epigenetic content, as an immortalized RPE cell line (e.g., the ARPE-19 cell line (ATCC® CRL-2302™)). Other exemplary strains of RPE cells include ARPE-19-SEAP-2-neo cells, RPE-J cells, and hTERT RPE-1 cells. In an embodiment, an RPE described herein is engineered, e.g., to have a new property, e.g., the cell is genetically modified by inserting at least one exogenous transcription unit into one or more of the OCR locations described herein.

“Sequence identity” or “percent identical”, when used herein to refer to two nucleotide sequences or two amino acid sequences, means the two sequences are the same within a specified region, or have the same nucleotides or amino acids at a specified percentage of nucleotide or amino acid positions within the specified when the two sequences are compared and aligned for maximum correspondence over a comparison window or designated region. Sequence identity may be determined using standard techniques known in the art including, but not limited to, any of the algorithms described in US Patent Application Publication No. 2017/02334455 A1. In an embodiment, the specified percentage of identical nucleotide or amino acid positions is at least about 80%, 85%, 90%, 91%, 92%, 93%, 94%, 95%, 96%, 97%, 98%, 99% or higher.

“Spherical” as used herein, mean a device (e.g., a hydrogel capsule or other particle) having a curved surface that forms a sphere (e.g., a completely round ball) or sphere-like shape, which may have waves and undulations, e.g., on the surface. Spheres and sphere-like objects can be mathematically defined by rotation of circles, ellipses, or a combination around each of the three perpendicular axes, a, b, and c. For a sphere, the three axes are the same length. Generally, a sphere-like shape is an ellipsoid (for its averaged surface) with semi-principal axes within 10%, or 5%, or 2.5% of each other. The diameter of a sphere or sphere-like shape is the average diameter, such as the average of the semi-principal axes.

“Spheroid”, as that term is used herein to refer to a device (e.g., a hydrogel capsule or other particle), means the device has (i) a perfect or classical oblate spheroid or prolate spheroid shape or (ii) has a surface that roughly forms a spheroid, e.g., may have waves and undulations and/or may be an ellipsoid (for its averaged surface) with semi-principal axes within 100% of each other.

“Subject” as used herein refers to a human or non-human animal. In an embodiment, the subject is a human (i.e., a male or female) of any age group, e.g., a pediatric human subject (e.g., infant, child, adolescent) or adult human subject (e.g., young adult, middle-aged adult, or senior adult)). In an embodiment, the subject is a non-human animal, for example, a mammal (e.g., a mouse, a dog, a primate (e.g., a cynomolgus monkey or a rhesus monkey). In an embodiment, the subject is a commercially relevant mammal (e.g., cattle, pig, horse, sheep, goat, cat, or dog) or a bird (e.g., a commercially relevant bird such as a chicken, duck, goose, or turkey). In certain embodiments, the animal is a mammal. The animal may be a male or female and at any stage of development. A non-human animal may be a transgenic animal.

“Transcription unit” means a DNA sequence, e.g., present in an exogenous nucleie acid, that comprises at least a promoter sequence operably linked to a coding sequence, and may also comprise one or more additional elements that control or enhance transcription of the coding sequence into RNA molecules or translation of the RNA molecules into polypeptide molecules. In some embodiments, a transcription unit also comprises a polyadenylation (polyA) signal sequence and polyA site. In an embodiment, a transcription unit is present as an exogenous sequence integrated in one or more of die specific OCR insertion locations described herein.

“Treatment,” “treat,” and “treating” as used herein refers to one or more of reducing, reversing, alleviating, delaying the onset of, or inhibiting the progress of one or more of a symptom, manifestation, or underlying cause, of a disease (e.g., Fabry disease or hemophilia A). In an embodiment, treating comprises reducing, reversing, alleviating, delaying the onset of, or inhibiting the progress of a symptom or condition associated with the disease. In an embodiment, treating comprises increasing levels of a therapeutic polypeptide in at least one tissue of a subject in need thereof, e.g., in one or more of plasma, liver, kidney and heart. In some embodiments, “treatment,” “treat,” and “treating” require that signs or symptoms associated with the disease or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., due to a history of symptoms and/or genetic or other susceptibility factors). Treatment may also be continued after symptoms have resolved, for example, to delay or prevent recurrence. In some embodiments, treatment comprises prevention and in other embodiments it does not.

“Wild-type” (wt) refers to the natural form, including sequence, of a polynucleotide, polypeptide or protein in a species. A wild-type form is distinguished from a mutant form of a polynucleotide, polypeptide or protein arising from genetic mutation(s).

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”), 1 to 10 carbon atoms (“C₁-C₁₀ alkyl”), 1 to 8 carbon atoms (“C₁-C₈ alkyl”), 1 to 6 carbon atoms (“C₁-C₆ alkyl”), 1 to 5 carbon atoms (“C₁-C₅ alkyl”), 1 to 4 carbon atoms (“C₁-C₄alkyl”), 1 to 3 carbon atoms (“C₁-C₃ alkyl”), 1 to 2 carbon atoms (“C₁-C₂ alkyl”), or 1 carbon atom (“C₁ alkyl”). In some embodiments, an alkyl group has 2 to 6 carbon atoms (“C₂-C₆ alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 12 carbon atoms (“C₂-C₁₂alkenyl”), 2 to 10 carbon atoms (“C₂-C₁₀ alkenyl”), 2 to 8 carbon atoms (“C₂-C₈ alkenyl”), 2 to 6 carbon atoms (“C₂-C₆ alkenyl”), 2 to 5 carbon atoms (“C₂-C₅ alkenyl”), 2 to 4 carbon atoms (“C₂-C₄ alkenyl”), 2 to 3 carbon atoms (“C₂-C₃ alkenyl”), or 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 12 carbon atoms (“C₂-C₁₂ alkynyl”), 2 to 10 carbon atoms (“C₂-C₁₀ alkynyl”), 2 to 8 carbon atoms (“C₂-C₈ alkynyl”), 2 to 6 carbon atoms (“C₂-C₆ alkynyl”), 2 to 5 carbon atoms (“C₂-C₅ alkynyl”), 2 to 4 carbon atoms (“C₂-C₄ alkynyl”), 2 to 3 carbon atoms (“C₂-C₃ alkynyl”), or 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. An alkylene, alkenylene, alkynylene, or heteroalkylene group may be described as, e.g., a C₁-C₆-membered alkylene, C₂-C₆-membered alkenylene, C₁-C₆-membered alkynylene, or C₁-C₆-membered heteroalkylene, wherein the term “membered” refers to the non-hydrogen atoms within the moiety. In the case of heteroalkylene groups, heteroatoms can also occupy either or both chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′— and —R′C(O)₂—.

As used herein, “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents.

As used herein, “heteroaryl” refers to a radical of a 5-10 membered monocyclic or bicyclic 4n+2 aromatic ring system (e.g., having 6 or 10π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, the terms “arylene” and “heteroarylene,” alone or as part of another substituent, mean a divalent radical derived from an aryl and heteroaryl, respectively.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈ cycloalkyl”), 3 to 6 ring carbon atoms (“C₃-C₆ cycloalkyl”), or 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₈), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro [4.5] decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, piperazinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl or thiomorpholinyl-1,1-dioxide. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Amino” as used herein refers to the radical —NR⁷⁰R⁷¹, wherein R⁷⁰ and R⁷¹ are each independently hydrogen, C₁-C₈ alkyl, C₃-C₁₀ cycloalkyl, C₄-C₁₀ heterocyclyl, C₆-C₁₀ aryl, and C₅-C₁₀ heteroaryl. In some embodiments, amino refers to NH₂.

As used herein, “cyano” refers to the radical —CN.

As used herein, “halo” or “halogen,” independently or as part of another substituent, mean, unless otherwise stated, a fluorine (F), chlorine (Cl), bromine (Br), or iodine (I) atom.

As used herein, “hydroxy” refers to the radical —OH.

Alkyl, alkenyl, alkynyl, heteroalkyl, cycloalkyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Compounds of Formula (I) described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E.L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

Compounds of Formula (I) described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

The term “pharmaceutically acceptable salt” is meant to include salts of the active compounds that are prepared with relatively nontoxic acids or bases, depending on the particular substituents found on the compounds described herein. When compounds of Formula (I) used to prepare devices of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds used in the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, e.g., Berge et al, Journal of Pharmaceutical Science 66: 1-19 (1977)). Certain specific compounds used in the devices of the present disclosure (e.g., a particle, a hydrogel capsule) contain both basic and acidic functionalities that allow the compounds to be converted into either base or acid addition salts. These salts may be prepared by methods known to those skilled in the art. Other pharmaceutically acceptable carriers known to those of skill in the art are suitable for use in the present disclosure.

Devices of the present disclosure may contain a compound of Formula (I) in a prodrug form. Prodrugs are those compounds that readily undergo chemical changes under physiological conditions to provide the compounds useful to mitigate the FBR to devices of the present disclosure. Additionally, prodrugs can be converted to useful compounds of Formula (I) by chemical or biochemical methods in an ex vivo environment.

Certain compounds of Formula (I) described herein can exist in unsolvated forms as well as solvated forms, including hydrated forms. In general, the solvated forms are equivalent to unsolvated forms and are encompassed within the scope of the present disclosure. Certain compounds of Formula (I) described herein may exist in multiple crystalline or amorphous forms. In general, all physical forms are equivalent for the uses contemplated by the present disclosure and are intended to be within the scope of the present disclosure.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0.

The term “tautomer” as used herein refers to compounds that are interchangeable forms of a compound structure, and that vary in the displacement of hydrogen atoms and electrons. Thus, two structures may be in equilibrium through the movement of R electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

The symbol “ [symbol] ” as used herein refers to a connection to an entity, e.g., a polymer (e.g., hydrogel-forming polymer such as alginate) or surface of an implantable element (e.g., a particle, device (e.g., a hydrogel capsule) or material). The connection represented by “ [symbol] ” may refer to direct attachment to the entity, e.g., a polymer or an implantable element (e.g., a device), or may refer to linkage to the entity through an attachment group. An “attachment group,” as described herein, refers to a moiety for linkage of a compound of Formula (I) to an entity (e.g., a polymer or an implantable element as described herein), and may comprise any attachment chemistry known in the art. A listing of exemplary attachment groups is outlined in Bioconjugate Techniques (3^{rd} ed, Greg T. Hermanson, Waltham, MA: Elsevier, Inc, 2013), which is incorporated herein by reference in its entirety. In some embodiments, an attachment group comprises alkyl, alkenyl, alkynyl, heteroalkyl, cycloalkyl, heterocyclyl, aryl, heteroaryl, —C(O)—, —OC(O)—, —N(R^{C})—, —N(R^{C})C(O)—, —C(O)N(R^{C})—, —N(R^{C})N(R^{D})—, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, wherein each of R^{A}, R^{C}, R^{D}, R^{F}, R^{G}, x and y is independently as described herein. In some embodiments, an attachment group comprises an amine, ketone, ester, amide, alkyl, alkenyl, alkynyl, or thiol. In some embodiments, an attachment group is a cross-linker. In some embodiments, the attachment group is —C(O)(C₁-C₆-alkylene)-, wherein alkylene is substituted with R¹, and R¹ is as described herein. In some embodiments, the attachment group is —C(O)(C₁-C₆-alkylene)-, wherein alkylene is substituted with 1-2 alkyl groups (e.g., 1-2 methyl groups). In some embodiments, the attachment group is —C(O)C(CH₃)₂—. In some embodiments, the attachment group is —C(O)(methylene)-, wherein methylene is substituted with 1-2 alkyl groups (e.g., 1-2 methyl groups). In some embodiments, the attachment group is —C(O)CH(CH₃)—. In some embodiments, the attachment group is —C(O)C(CH₃)₂—.

### Genomic Insertion Sites and Exogenous Transcription Units

The genetically modified human cells (e.g., genetically modified human RPE cells) of the present disclosure comprise at least one exogenous transcription unit stably integrated into one or more of 23 target OCRs in the genome. The 5′ and 3′ boundaries of a target OCR may be defined in relation to: (i) a human reference genome sequence assembly, e.g., the Genome Reference Consortium Human Reference 38 patch 12 (Assembly: GCA_0000014505.27, referred to herein as GRCh38.p12), which assembly is based at the European Molecular Biology Laboratory's European Bioinformatics Institute (EMBL-EBI) (Cambridge, United Kingdom) and/or (ii) one of the OCR nucleotide sequences recited in SEQ ID NO:1-SEQ ID NO:23.

In an embodiment, an exogenous transcription unit is inserted in the target genomic OCR(s) of the genetically modified human cell anywhere between the two nucleotide positions that define the 5′ and 3′ boundaries of the target OCR(s), e.g., within any of SEQ ID NOs:1-23. In some embodiments, the exogenous transcription unit is inserted between two contiguous nucleotide positions in the reference genomic sequence. In other embodiments, the two genomic nucleotide positions on either side of the inserted transcription unit are non-contiguous in the target OCR, e.g., the insertion event results in a deletion of one or more nucleotides in the OCR. In an embodiment, the two nucleotide positions that define an insertion site are within a nucleotide sequence that shares at least 90% sequence identity with any of SEQ ID NOs:1-23. In an embodiment, the shared sequence identity of a target OCR in the genetically modified cell line and the corresponding reference sequence in SEQ ID NO:1-23 is 91%, 92%, 93%, 94%, 95%, 96%, 97%, 98%, 99% or greater. These sequences may be used in combination with a targeted genome editing technique to insert the transcription unit into a human cell, e.g., an epithelial cell, at a desired site(s) in the target OCR(s), e.g., in the genomic insertion sites described in Table 1. The targeted genome editing technique may be any technique known in the art, e.g., techniques that employ site directed nucleases such as CRISPR-Cas, zinc finger nucleases, transcription activator-like effector nucleases (TALENs), and meganucleases.

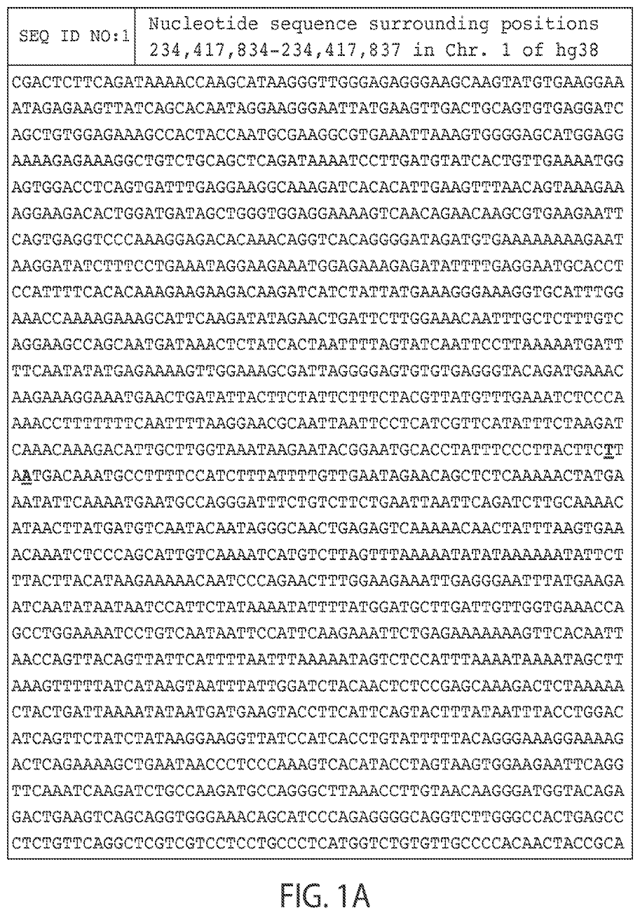

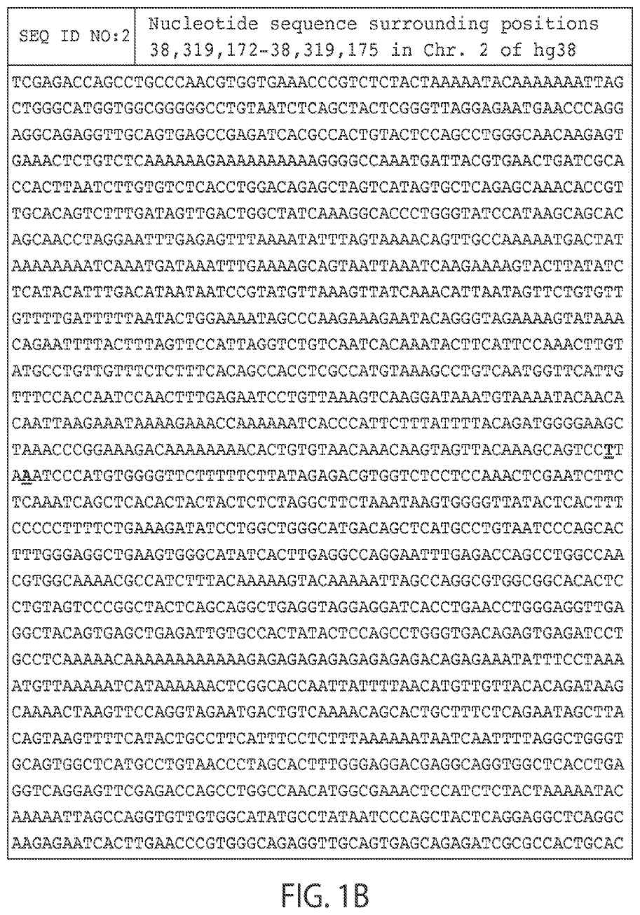

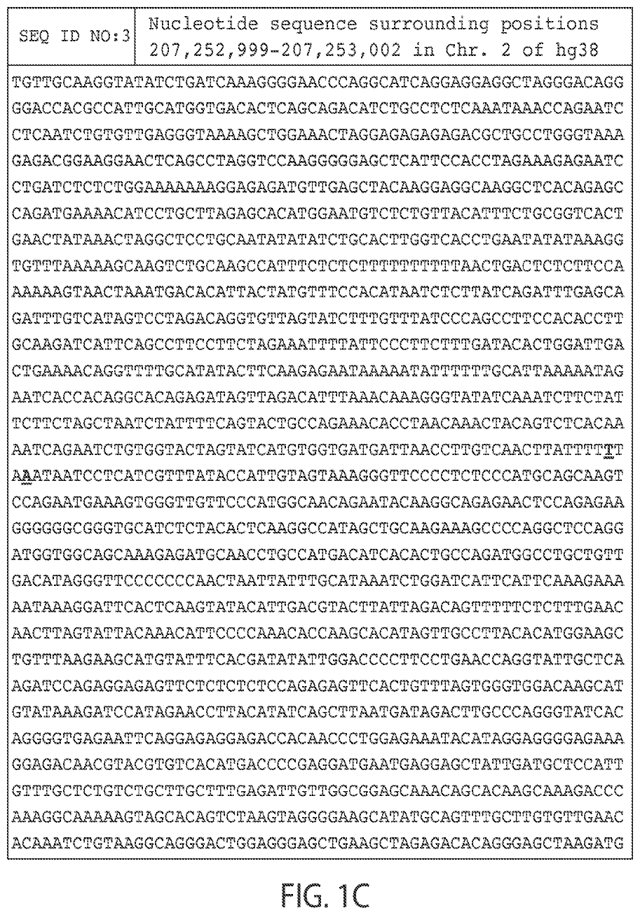

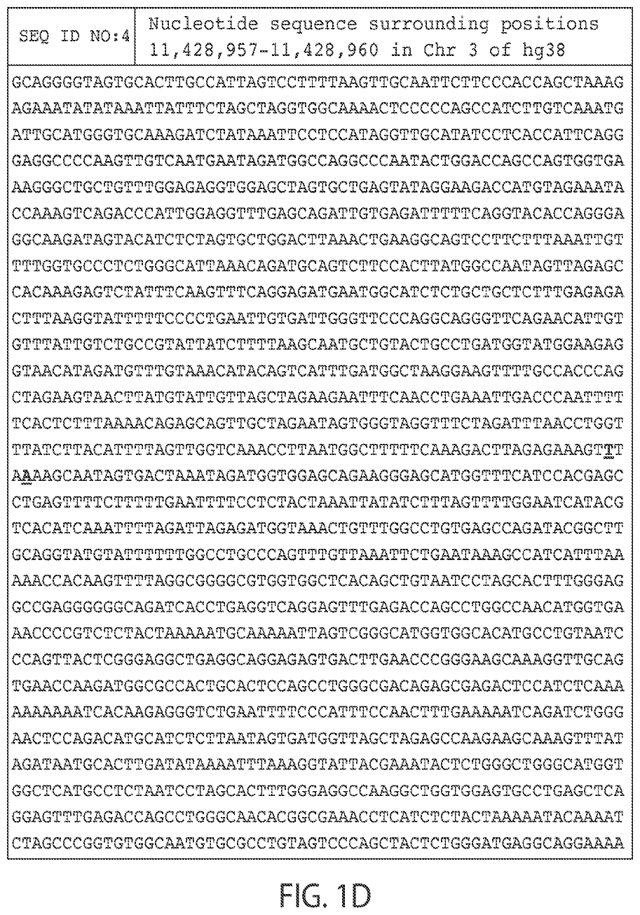

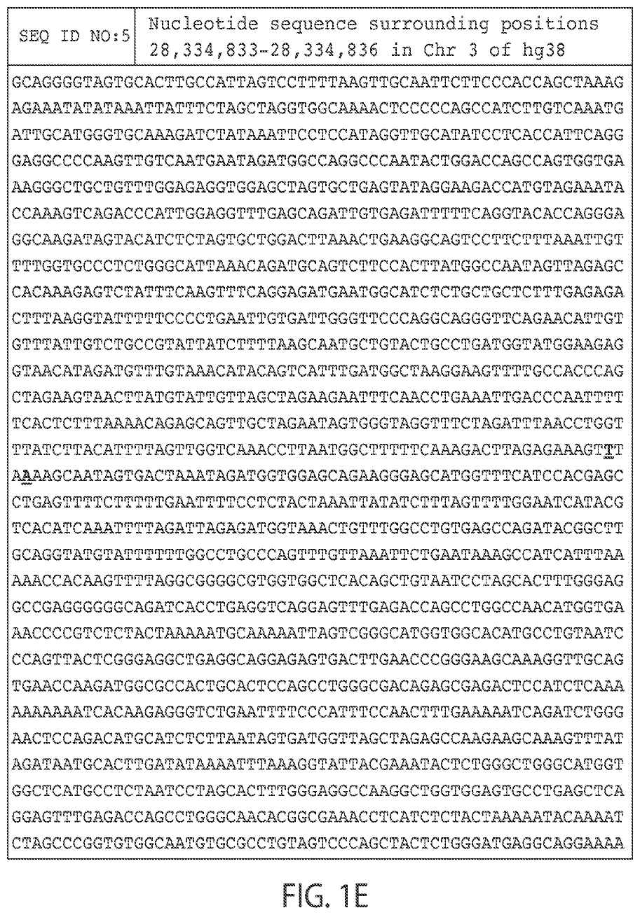

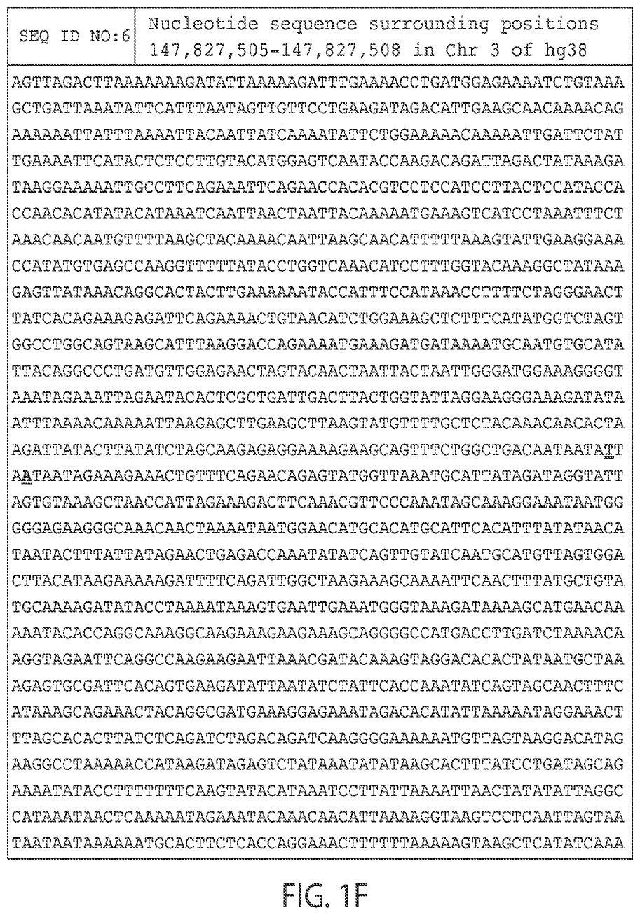

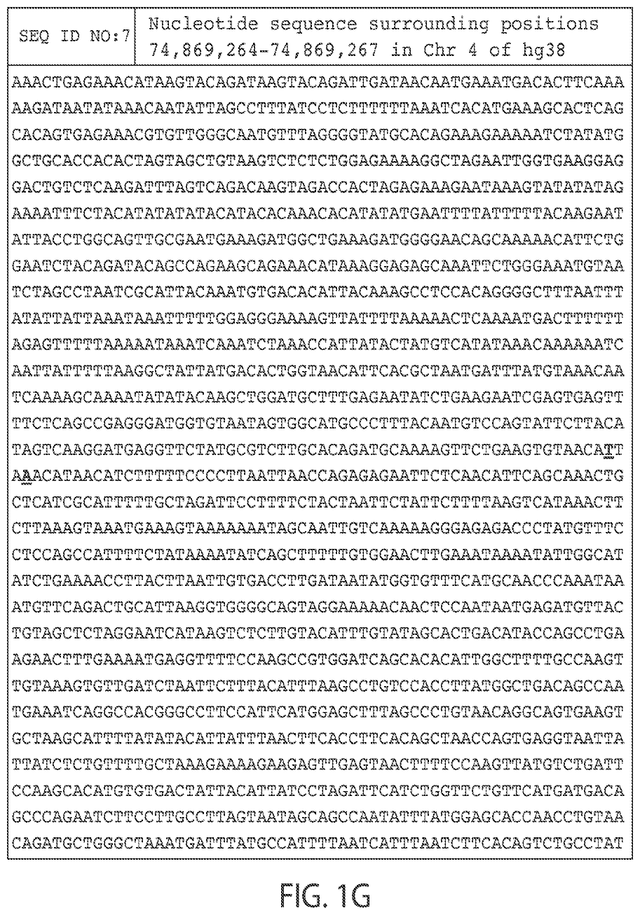

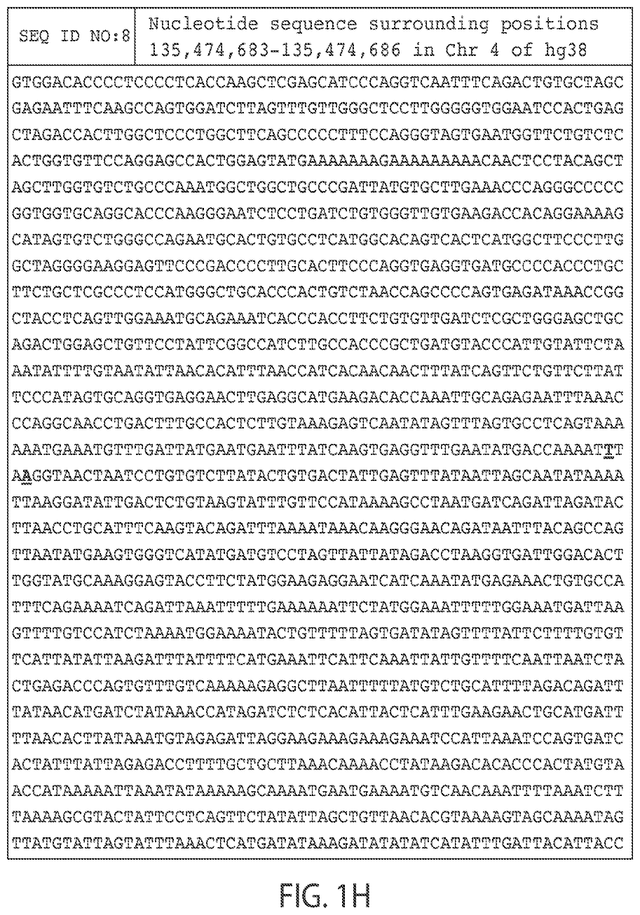

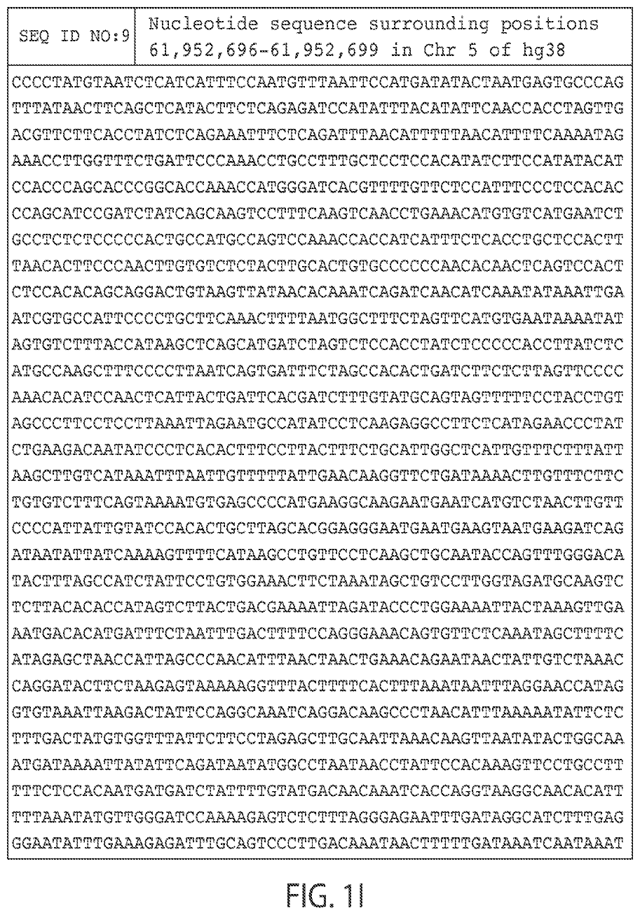

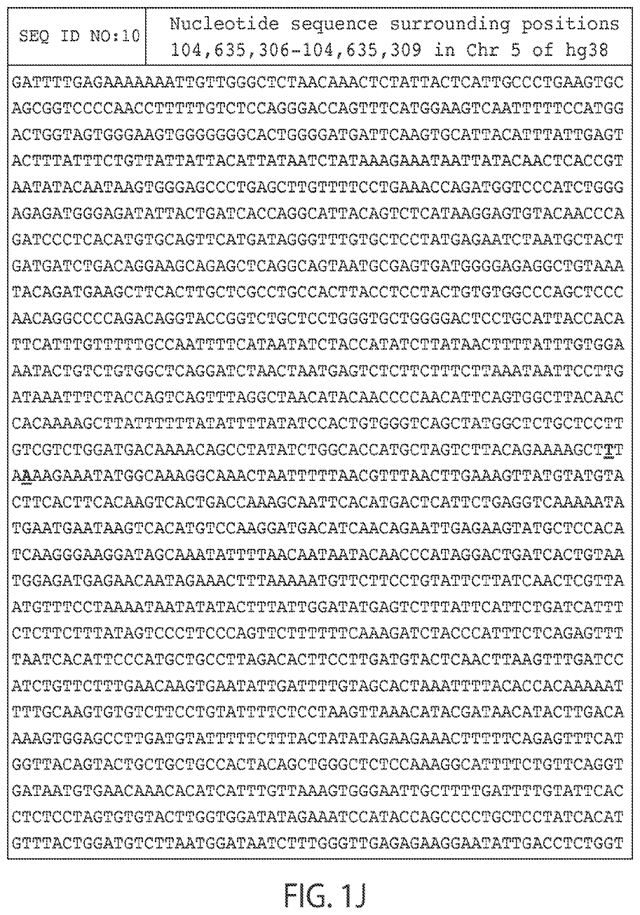

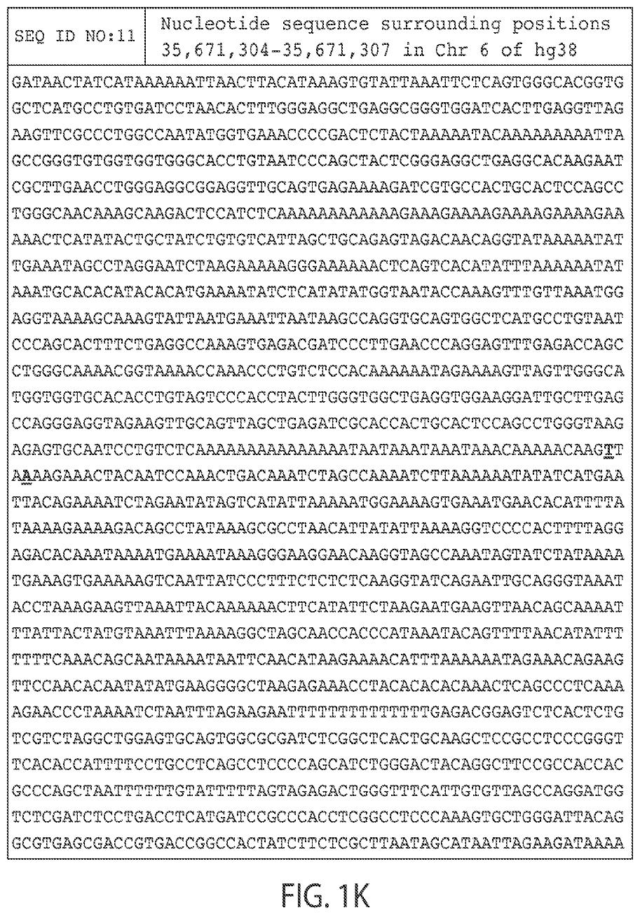

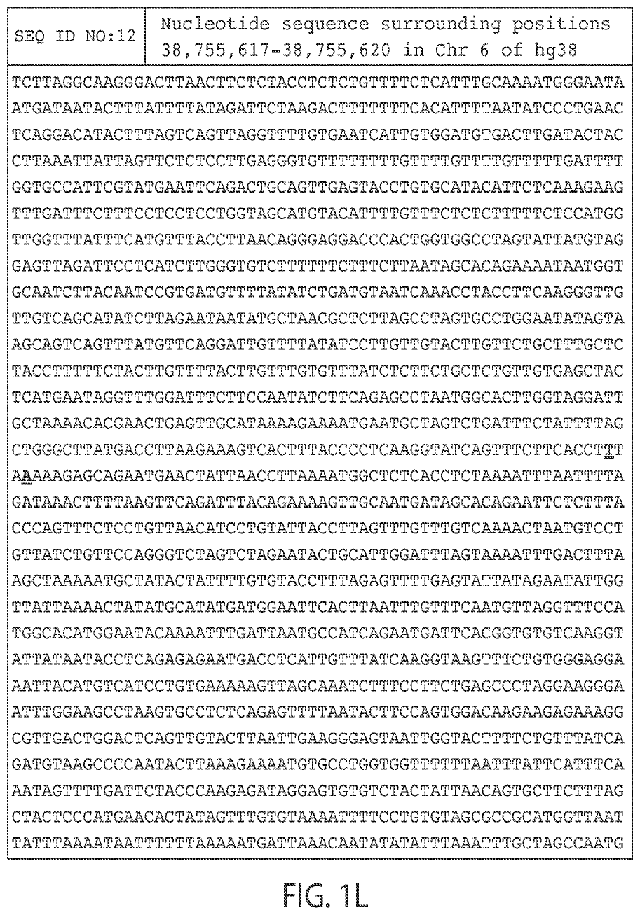

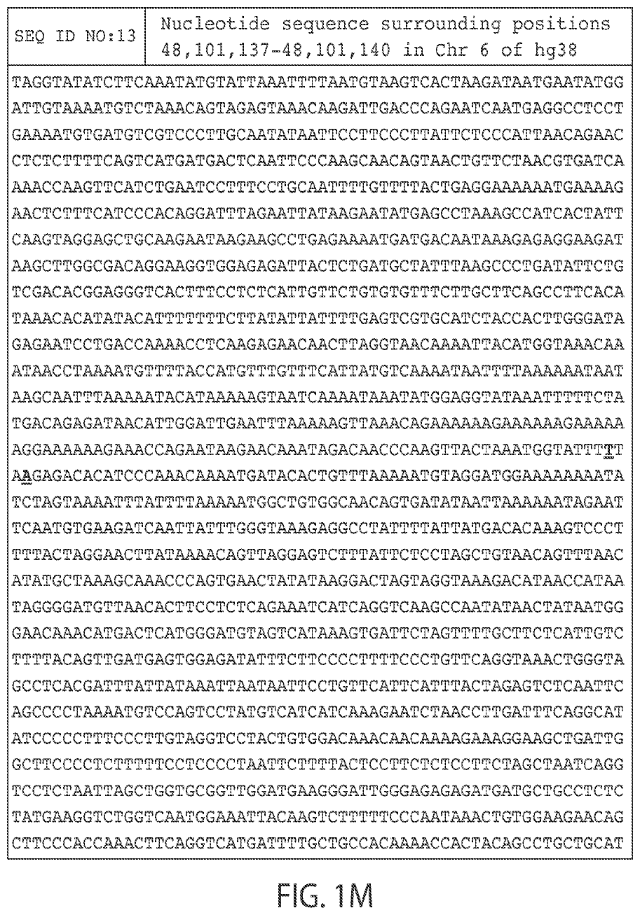

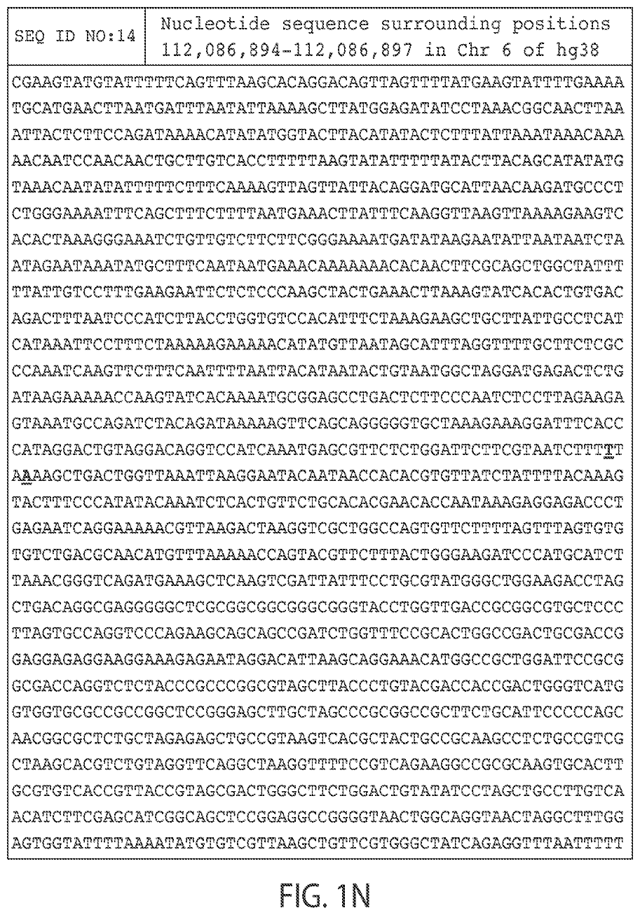

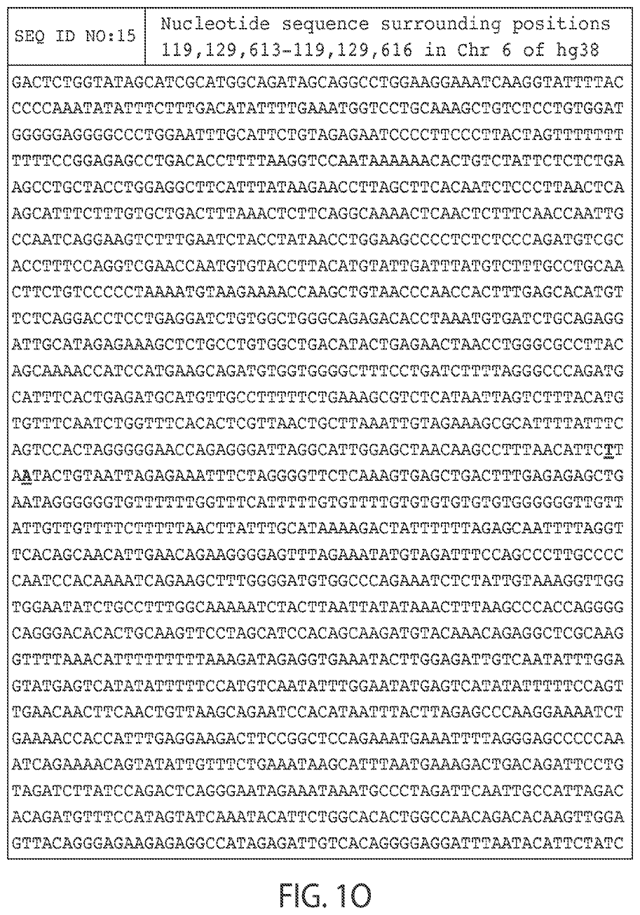

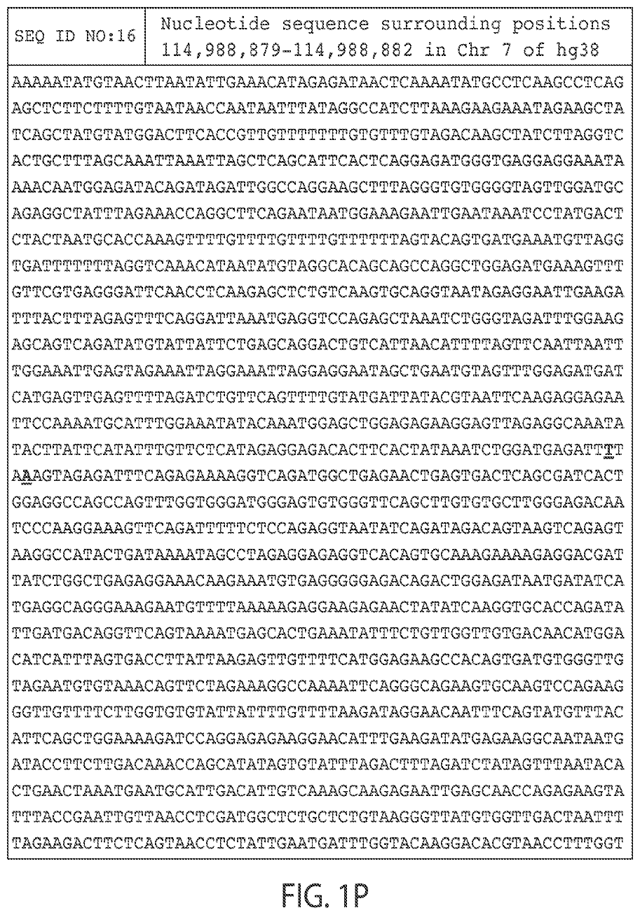

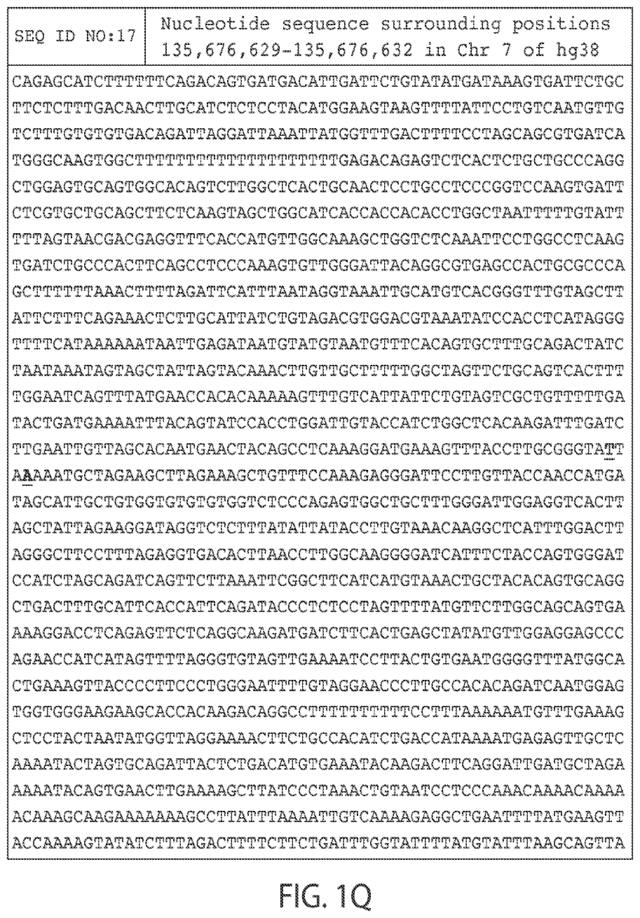

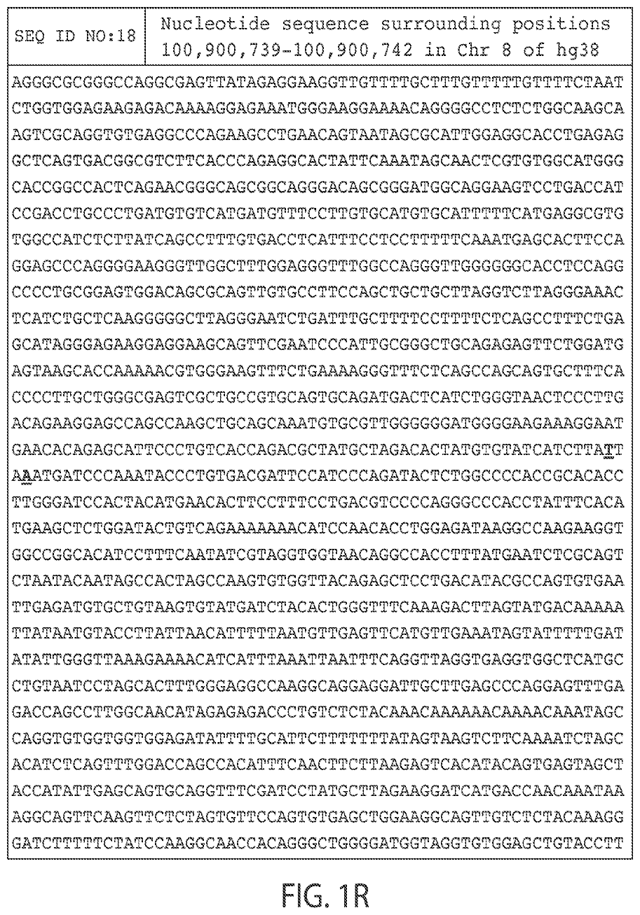

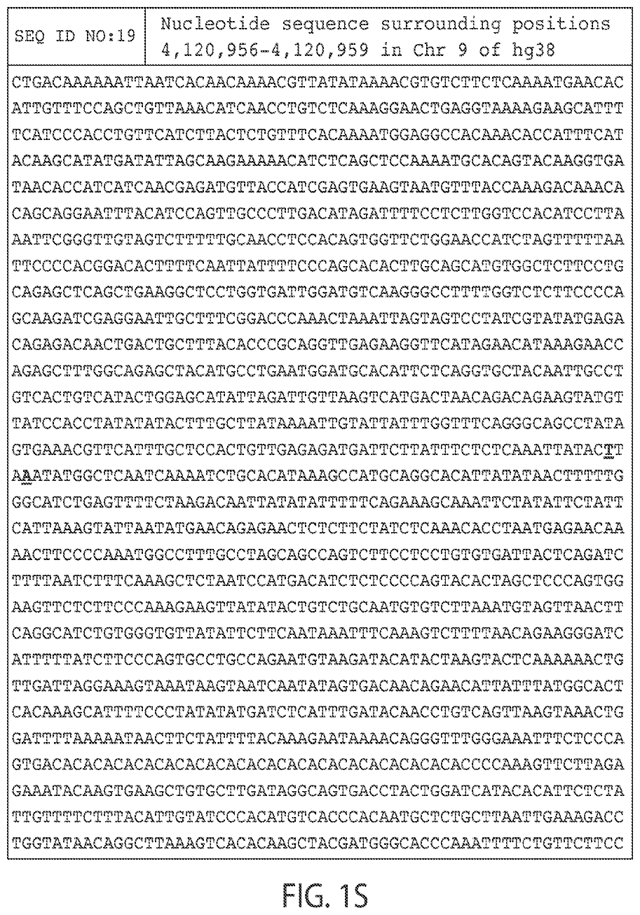

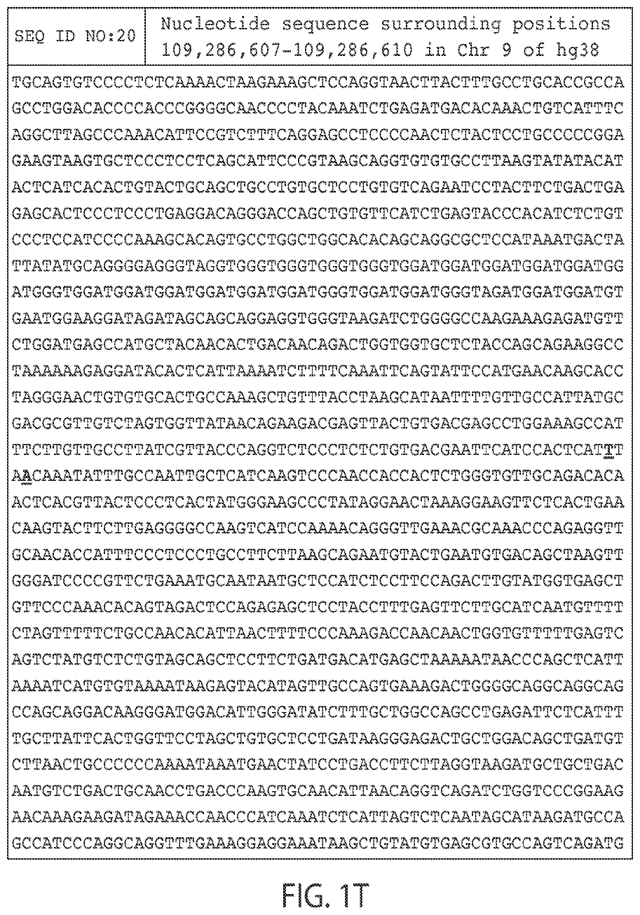

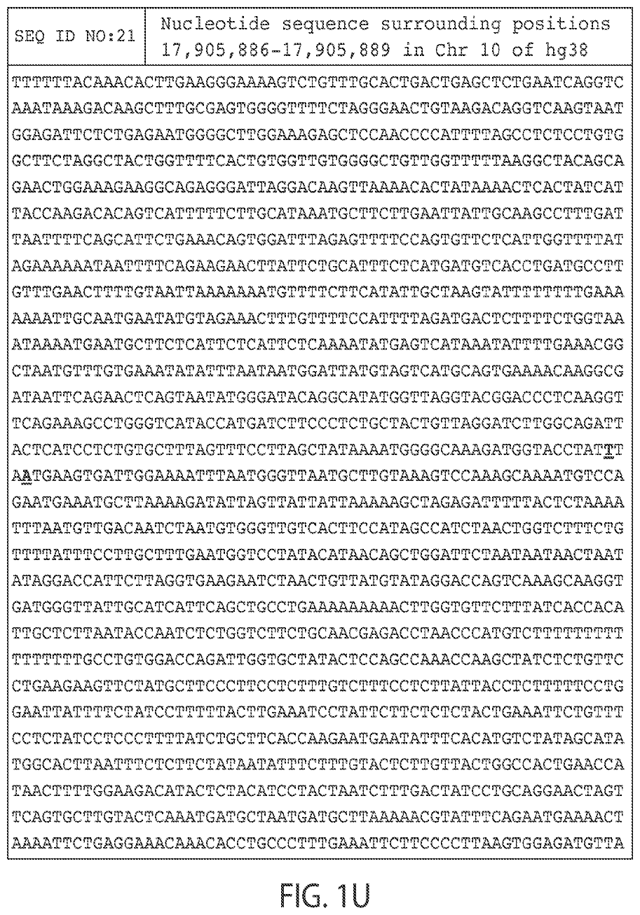

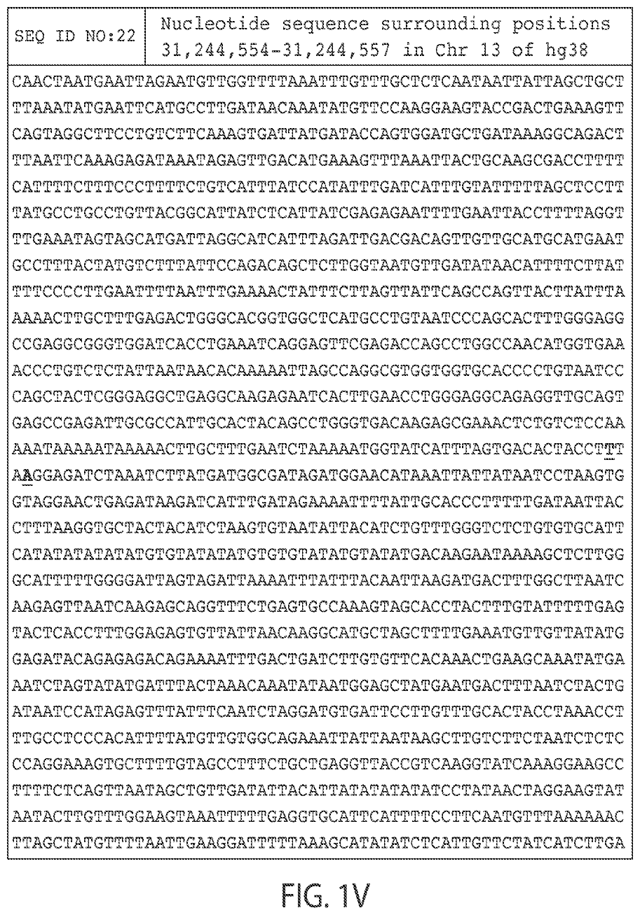

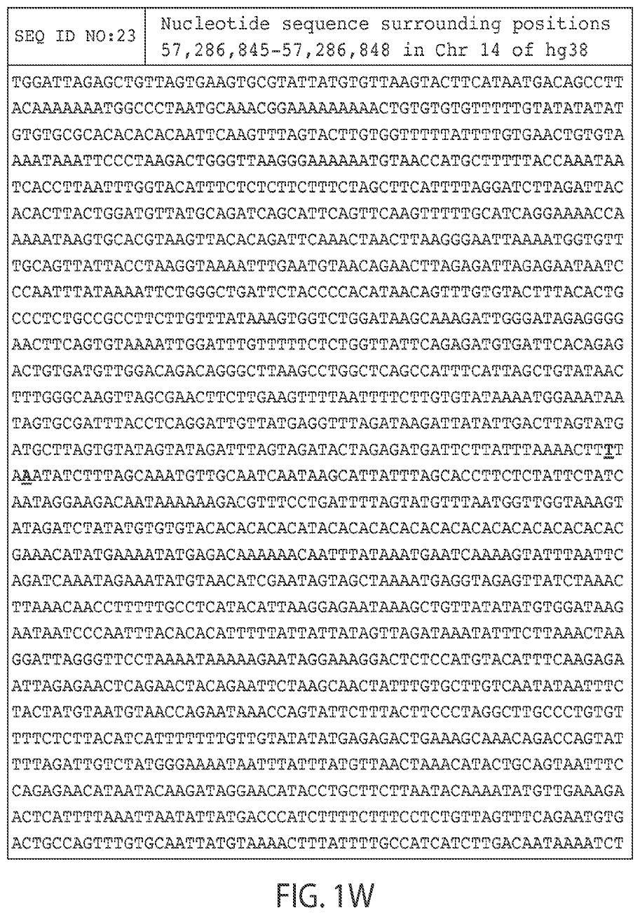

In some embodiments, a genomic insertion site (GIS) for an exogenous transcription unit is located in Chr 1 between two nucleotide positions corresponding to positions x₁ and y₁ in SEQ ID NO:1, wherein x₁ and y₁ are selected from the group consisting of: 100 and 1700; 200 and 1600; 400 and 1400; 800 and 1000; 850 and 950; and 875 and 925. In an embodiment, the 5′ and 3′ boundaries of the GIS are defined by two nucleotide positions corresponding to the 5′ and 3′ boundary positions shown in FIG. 1A.

In some embodiments, a Chr 2 GIS for an exogenous transcription unit is located between two nucleotide positions corresponding to positions x₂ and y₂ in SEQ ID NO:2 or in SEQ ID NO:3, wherein x₁ and y₁ are selected from the group consisting of: 100 and 1700; 200 and 1600; 400 and 1400; 800 and 1000; 850 and 950; and 875 and 925. In an embodiment, the 5′ and 3′ boundaries of the GIS are defined by two nucleotide positions corresponding to the 5′ and 3′ boundary positions shown in FIG. 1B or FIG. 1C.

In some embodiments, a Chr 3 GIS for an exogenous transcription unit is located between two nucleotide positions corresponding to positions x₃ and y₃ in SEQ ID NO:4, SEQ ID NO:5 or SEQ ID NO:6, wherein x₃ and y₃ are selected from the group consisting of: 100 and 1700; 200 and 1600; 400 and 1400; 800 and 1000; 850 and 950; and 875 and 925. In an embodiment, the 5′ and 3′ boundaries of the GIS are defined by two nucleotide positions corresponding to the 5′ and 3′ boundary positions shown in FIG. 1D, FIG. 1E or FIG. 1F.

In some embodiments, a Chr 4 GIS for an exogenous transcription unit is located between two nucleotide positions corresponding to positions x₄ and y₄ in SEQ ID NO:7 or SEQ ID NO:8, wherein x₄ and y₄ are selected from the group consisting of: 100 and 1700; 200 and 1600; 400 and 1400; 800 and 1000; 850 and 950; and 875 and 925. In an embodiment, the 5′ and 3′ boundaries of the GIS are defined by two nucleotide positions corresponding to the 5′ and 3′ boundary positions shown in FIG. 1G or FIG. 1H.

In some embodiments, a Chr 5 GIS for an exogenous transcription unit is located between two nucleotide positions corresponding to positions x₅ and y₅ in SEQ ID NO:9 or SEQ ID NO:10, wherein x₅ and y₅ are selected from the group consisting of: 100 and 1700; 200 and 1600; 400 and 1400; 800 and 1000; 850 and 950; and 875 and 925. In an embodiment, the 5′ and 3′ boundaries of the GIS are defined by two nucleotide positions corresponding to the 5′ and 3′ boundary positions shown in FIG. 1I or FIG. 1J.

In some embodiments, a Chr 6 GIS for an exogenous transcription unit is located between two nucleotide positions corresponding to positions x₆ and y₆ in SEQ ID NO:11, SEQ ID NO:12, SEQ ID NO:13, SEQ ID NO:14 or SEQ ID NO:15, wherein x₆ and y₆ are selected from the group consisting of: 100 and 1700; 200 and 1600; 400 and 1400; 800 and 1000; 850 and 950; and 875 and 925. In an embodiment, the 5′ and 3′ boundaries of the GIS are defined by two nucleotide positions corresponding to the 5′ and 3′ boundary positions shown in FIG. 1K, FIG. 1L, FIG. 1M, FIG. 1N or FIG. 1O.

In some embodiments, a Chr 7 GIS for an exogenous transcription unit is located between two nucleotide positions corresponding to positions x₇ and y₇ in SEQ ID NO:16 or SEQ ID NO:17, wherein x₇ and y₇ are selected from the group consisting of: 100 and 1700; 200 and 1600; 400 and 1400; 800 and 1000; 850 and 950; and 875 and 925. In an embodiment, the 5′ and 3′ boundaries of the GIS are defined by two nucleotide positions corresponding to the 5′ and 3′ boundary positions shown in FIG. 1P or FIG. 1Q.

In some embodiments, a Chr 8 GIS for an exogenous transcription unit is located between two nucleotide positions corresponding to positions x₈ and y₈ in SEQ ID NO:18, wherein x₇ and y₇ are selected from the group consisting of: 100 and 1700; 200 and 1600; 400 and 1400; 800 and 1000; 850 and 950; and 875 and 925. In an embodiment, the 5′ and 3′ boundaries of the GIS are defined by two nucleotide positions corresponding to the 5′ and 3′ boundary positions shown in FIG. 1R.

In some embodiments, a Chr 9 GIS for an exogenous transcription unit is located between two nucleotide positions corresponding to positions x₉ and y₉ in SEQ ID NO:19 or SEQ ID NO:20, wherein x₉ and y₉ are selected from the group consisting of: 100 and 1700; 200 and 1600; 400 and 1400; 800 and 1000; 850 and 950; and 875 and 925. In an embodiment, the 5′ and 3′ boundaries of the GIS are defined by two nucleotide positions corresponding to the 5′ and 3′ boundary positions shown in FIG. 1S or FIG. 1T.

In some embodiments, a Chr 10 GIS for an exogenous transcription unit is located between two nucleotide positions corresponding to positions x₁₀ and y₁₀ in SEQ ID NO:21, wherein x₁₀ and y₁₀ are selected from the group consisting of: 100 and 1700; 200 and 1600; 400 and 1400; 800 and 1000; 850 and 950; and 875 and 925. In an embodiment, the 5′ and 3′ boundaries of the GIS are defined by two nucleotide positions corresponding to the 5′ and 3′ boundary positions shown in FIG. 1U.

In some embodiments, a Chr 13 GIS for an exogenous transcription unit is located between two nucleotide positions corresponding to positions x₁₃ and y₁₃ in SEQ ID NO:22, wherein x₁₃ and y₁₃ are selected from the group consisting of: 100 and 1700; 200 and 1600; 400 and 1400; 800 and 1000; 850 and 950; and 875 and 925. In an embodiment, the 5′ and 3′ boundaries of the GIS are defined by two nucleotide positions corresponding to the 5′ and 3′ boundary positions shown in FIG. 1V.

In some embodiments, a Chr 14 GIS for an exogenous transcription unit is located between two nucleotide positions corresponding to positions x₈ and y₈ in SEQ ID NO:23, wherein x₁₄ and y₁₄ are selected from the group consisting of: 100 and 1700; 200 and 1600; 400 and 1400; 800 and 1000; 850 and 950; and 875 and 925. In an embodiment, the 5′ and 3′ boundaries of the GIS are defined by two nucleotide positions corresponding to the 5′ and 3′ boundary positions shown in FIG. 1W.

In an embodiment, an exogenous transcription unit is inserted into one, two, three, four, five, six, seven, eight, nine, ten or more of the target OCRs in Chr 1, Chr 2, Chr 3, Chr 4, Chr 5, Chr 6, Chr 7, Chr 8, Chr 9, Chr 10, Chr 13 and Chr 14 of the genetically modified cell.

In an embodiment the exogenous transcription unit is inserted into each of the five OCRs defined in SEQ ID NO:1, SEQ ID NO:4, SEQ ID NO:14, SEQ ID NO:16 and SEQ ID NO:20. In an embodiment the exogenous transcription unit is inserted into each of the 18 OCRs defined in SEQ ID NO:2, SEQ ID NO:3, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:15, SEQ ID NO:17, SEQ ID NO:18, SEQ ID NO:19, SEQ ID NO:21, SEQ ID NO:22 and SEQ ID NO:23. In an embodiment, the exogenous transcription unit is inserted into these 18 OCRs and at least one additional OCR that does not comprise any of SEQ ID NO:1-23.

The transcription unit in each insertion site may encode the same or different substance, e.g., a polypeptide. Two or more transcription units may be inserted in tandem at a single site in a target OCR, and may encode the same or different substances. In an embodiment, the promoter in the upstream transcription unit is different that the promoter in the downstream transcription unit.

The promoter sequence in each inserted transcription unit may be for any promoter capable of driving expression of a coding sequence operably linked to the promoter in the genetically modified cell. In an embodiment, the promoter sequence consists essentially of, or consists of, SEQ ID NO:24 or a nucleotide sequence that is substantially identical to SEQ ID NO:24, e.g., is at least 95%, 96%, 97%, 98%, 99% or more identical to SEQ ID NO:24. In an embodiment, the promoter consists of SEQ ID NO:24. In an embodiment, the promoter sequence consists essentially of, or consists of, SEQ ID NO:25 or a nucleotide sequence that is substantially identical to SEQ ID NO:25, e.g., is at least 95%, 96%, 97%, 98%, 99% or more identical to SEQ ID NO:25. In an embodiment, the promoter consists of SEQ ID NO:25.

The coding sequence in each inserted transcription unit may be operably linked to a polyA signal sequence, which sequence may be the same or different in each transcription unit. In an embodiment, the polyA signal sequence consists essentially of, or consists of, SEQ ID NO:26 or a nucleotide sequence that is substantially identical to SEQ ID NO:26, e.g., is at least 95%, 96%, 97%, 98%, 99% or more identical to SEQ ID NO:26. In an embodiment, the polyA signal sequence consists of SEQ ID NO:26.

In some embodiments, the exogenous transcription unit encodes a therapeutic polypeptide (e.g., a protein), such as a clotting factor, growth factor, hormone, enzyme, cytokine (e.g., a pro-inflammatory cytokine or an anti-inflammatory cytokine), cytokine receptor, chimeric protein, fusion protein or lipoprotein. The polypeptide encoded by the exogenous transcription unit may have a naturally occurring amino acid sequence or may contain a variant of the naturally occurring sequence. The variant can be a non-naturally occurring or naturally occurring amino acid substitution, mutation, deletion or addition relative to the reference (e.g., naturally occurring) sequence. The naturally occurring amino acid sequence may be a polymorphic variant. The naturally occurring amino acid sequence can be a human or a non-human amino acid sequence. In some embodiments, the naturally occurring amino acid sequence is a human sequence. In some embodiments, the therapeutic polypeptide has about 2, 3, 4, 5, 6, 7, 8, 9, 10, 12, 14, 16, 18, 20, 25, 30, 35, 40, 45, or less than 50 amino acids. In some embodiments, the polypeptide has an average molecular weight of 5 kD, 10 kD, 25 kD, 50 kD, 100 kD, 150 kD, 200 kD, 250 kD, 500 kD, or more.

In some embodiments, the polypeptide is a hormone. Exemplary hormones include anti-diuretic hormone (ADH), oxytocin, growth hormone (GH), prolactin, growth hormone-releasing hormone (GHRH), thyroid stimulating hormone (TSH), thyrotropin-release hormone (TRH), adrenocorticotropic hormone (ACTH), follicle-stimulating hormone (FSH), luteinizing hormone (LH), luteinizing hormone-releasing hormone (LHRH), thyroxine, calcitonin, parathyroid hormone (PTH), aldosterone, cortisol, epinephrine, glucagon, insulin, estrogen, progesterone, and testosterone. In some embodiments, the polypeptide is insulin (e.g., insulin A-chain, insulin B-chain, or proinsulin). In some embodiments, the polypeptide is a growth hormone, such as human growth hormone (hGH), recombinant human growth hormone (rhGH), bovine growth hormone, methionine-human growth hormone, des-phenylalanine human growth hormone, and porcine growth hormone.

In some embodiments, the polypeptide is a growth factor, e.g., vascular endothelial growth factor (VEGF), nerve growth factor (NGF), platelet-derived growth factor (PDGF), fibroblast growth factor (FGF), epidermal growth factor (EGF), transforming growth factor (TGF), and insulin-like growth factor-I and -II (IGF-I and IGF-II).

In some embodiments, the polypeptide is a clotting factor or a coagulation factor, e.g., a blood clotting factor or a blood coagulation factor. In some embodiments, the polypeptide is involved in coagulation, i.e., the process by which blood is converted from a liquid to solid or gel. Exemplary clotting factors and coagulation factors include Factor I (e.g., fibrinogen), Factor II (e.g., prothrombin), Factor III (e.g., tissue factor), Factor V (e.g., proaccelerin, labile factor), Factor VI, Factor VII (e.g., stable factor, proconvertin), Factor VIII (e.g., antihemophilic factor A), Factor VIIIC, Factor IX (e.g., antihemophilic factor B), Factor X (e.g., Stuart-Prower factor), Factor XI (e.g., plasma thromboplastin antecedent), Factor XII (e.g., Hagerman factor), Factor XIII (e.g., fibrin-stabilizing factor), von Willebrand factor (vWF), prekallikrein, heparin cofactor II, high molecular weight kininogen (e.g., Fitzgerald factor), antithrombin III, and fibronectin. In some embodiments, the polypeptide is an anti-clotting factor, such as Protein C.

In some embodiments, the polypeptide is an immunoglobulin chain (heavy or light chain) or fragment thereof, comprising at least one immunoglobulin variable domain sequence, and optionally comprising an immunoglobulin Fc region. In an embodiment, the polypeptide a full-length immunoglobulin chain.

In some embodiments, the polypeptide is a cytokine or a cytokine receptor, or a chimeric protein including cytokines or their receptors, including, for example tumor necrosis factor alpha and beta, their receptors and their derivatives, renin; lipoproteins; colchicine; corticotrophin; vasopressin; somatostatin; lypressin; pancreozymin; leuprolide; alpha-1-antitrypsin; atrial natriuretic factor; lung surfactant; a plasminogen activator other than a tissue-type plasminogen activator (t-PA), for example a urokinase; bombesin; thrombin; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; chorionic gonadotropin; a microbial protein, such as beta-lactamase; DNase; inhibin; activin; receptors for hormones or growth factors; integrin; protein A or D; rheumatoid factors; platelet-derived growth factor (PDGF); epidermal growth factor (EGF); transforming growth factor (TGF) such as TGF-α and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; an interferon such as interferon-alpha (e.g., interferon.alpha.2A), -beta, -gamma, -lambda and consensus interferon; colony stimulating factors (CSFs), e.g., M-CSF, GM-CSF, and G-CSF; interleukins (ILs), e.g., IL-1, IL-2 to IL-10; superoxide dismutase; T-cell receptors; surface membrane proteins; decay accelerating factor; transport proteins; homing receptors; addressins; fertility inhibitors such as the prostaglandins; fertility promoters; regulatory proteins; antibodies (including fragments thereof) and chimeric proteins, such as immunoadhesins. Suitable polypeptides may be native or recombinant and include, e.g., fusion proteins.

Examples of a polypeptide that may be encoded by the exogenous transcription unit also include CCL1, CCL2 (MCP-1), CCL3 (MIP-1α), CCL4 (MIP-1β), CCL5 (RANTES), CCL6, CCL7, CCL8, CCL9 (CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1 (KC), CXCL2 (SDF1a), CXCL3, CXCL4, CXCL5, CXCL6, CXCL7, CXCL8 (IL8), CXCL9, CXCL10, CXCL11, CXCL12, CXCL13, CXCL14, CXCL15, CXCL16, CXCL17, CX3CL1, XCL1, XCL2, TNFA, TNFB (LTA), TNFC (LTB), TNFSF4, TNFSF5 (CD40LG), TNFSF6, TNFSF7, TNFSF8, TNFSF9, TNFSF10, TNFSF11, TNFSF13B, EDA, IL2, IL15, IL4, IL13, IL7, IL9, IL21, IL3, IL5, IL6, IL11, IL27, IL30, IL31, OSM, LIF, CNTF, CTF1, IL12a, IL12b, IL23, IL27, IL35, IL14, IL16, IL32, IL34, IL10, IL22, IL19, IL20, IL24, IL26, IL29, IFNL1, IFNL2, IFNL3, IL28, IFNA1, IFNA2, IFNA4, IFNA5, IFNA6, IFNA7, IFNA8, IFNA10, IFNA13, IFNA14, IFNA16, IFNA17, IFNA21, IFNB1, IFNK, IFNW1, IFNG, IL1A (IL1F1), IL1B (IL1F2), IL1Ra (IL1F3), IL1F5 (IL36RN), IL1F6 (IL36A), IL1F7 (IL37), IL1F8 (IL36B), IL1F9 (IL36G), IL1F10 (IL38), IL33 (IL1F11), IL18 (IL1G), IL17, KITLG, IL25 (IL17E), CSF1 (M-CSF), CSF2 (GM-CSF), CSF3 (G-CSF), SPP1, TGFB1, TGFB2, TGFB3, CCL3L1, CCL3L2, CCL3L3, CCL4L1, CCL4L2, IL17B, IL17C, IL17D, IL17F, AIMP1 (SCYE1), MIF, Areg, BC096441, Bmp1, Bmp10, Bmp15, Bmp2, Bmp3, Bmp4, Bmp5, Bmp6, Bmp7, Bmp8a, Bmp8b, C1qtnf4, Ccl21a, Ccl27a, Cd70, Cer1, Cklf, Clef1, Cmtm2a, Cmtm2b, Cmtm3, Cmtm4, Cmtm5, Cmtm6, Cmtm7, Cmtm8, Crlf1, Ctf2, Ebi3, Edn1, Fam3b, Fas1, Fgf2, Flt31, Gdf10, Gdf11, Gdf15, Gdf2, Gdf3, Gdf5, Gdf6, Gdf7, Gdf9, Gm12597, Gm13271, Gm13275, Gm13276, Gm13280, Gm13283, Gm2564, Gpi1, Grem1, Grem2, Grn, Hmgb1, Ifna11, Ifna12, Ifna9, Ifnab, Ifne, Il17a, Il23a, Il25, Il31, Iltifb, Inhba, Lefty1, Lefty2, Mstn, Nampt, Ndp, Nodal, Pf4, Pglyrp1, Prl7d1, Scg2, Scgb3a1, Slurp1, Spp1, Thpo, Tnfsf10, Tnfsf11, Tnfsf12, Tnfsf13, Tnfsf13b, Tnfsf14, Tnfsf15, Tnfsf18, Tnfsf4, Tnfsf8, Tnfsf9, Tslp, Vegfa, Wnt1, Wnt2, Wnt5a, Wnt7a, Xcll, epinephrine, melatonin, triiodothyronine, a prostaglandin, a leukotriene, prostacyclin, thromboxane, islet amyloid polypeptide, mullerian inhibiting factor or hormone, adiponectin, corticotropin, angiotensin, vasopressin, arginine vasopressin, atriopeptin, brain natriuretic peptide, calcitonin, cholecystokinin, cortistatin, enkephalin, endothelin, erythropoietin, follicle-stimulating hormone, galanin, gastric inhibitory polypeptide, gastrin, ghrelin, glucagon, glucagon-like peptide-1, gonadotropin-releasing hormone, hepcidin, human chorionic gonadotropin, human placental lactogen, inhibin, somatomedin, leptin, lipotropin, melanocyte stimulating hormone, motilin, orexin, oxytocin, pancreatic polypeptide, pituitary adenylate cyclase-activating peptide, relaxin, renin, secretin, somatostatin, thrombopoietin, thyrotropin, thyrotropin-releasing hormone, vasoactive intestinal peptide, androgen, alpha-glucosidase (also known as acid maltase), glycogen phosphorylase, glycogen debrancher enzyme, phosphofructokinase, phosphoglycerate kinase, phosphoglycerate mutase, lactate dehydrogenase, carnitine palymityl transferase, carnitine, and myoadenylate deaminase.

In some embodiments, the polypeptide is a replacement therapy or a replacement protein.

In some embodiments, the replacement therapy or replacement protein is a clotting factor or a coagulation factor, e.g., Factor VII, Factor VIII or Factor IX.

In some embodiments, the replacement therapy or replacement protein is an enzyme, e.g., alpha-galactosidase A (GLA), alpha-L-iduronidase (IDUA), glucocerebrosidase, or N-sulfoglucosamine sulfohydrolase (SGSH).

In some embodiments, the transcription unit encodes a GLA protein, e.g., a human GLA protein. In some embodiments, the GLA protein is a fusion protein that comprises, consists essentially of, or consists of SEQ ID NO:31.

In some embodiments, the genetically modified cell comprises an exogenous transcription unit encoding a GLA protein, e.g., the fusion protein of SEQ ID NO:31, inserted into more than one GIS defined herein. In an embodiment, a composition comprising a plurality of cells obtained by culturing this genetically modified cell is capable of secreting at least 5 picograms/cell/day in the media, as measured by any direct or indirect GLA activity assay known in the art, e.g., the enzymatic assay described in the Examples below. In an embodiment, the assay is performed substantially as follows. A suspension of about 400,000 cells (obtained by culturing the genetically modified cell) is added to wells of a 6-well plate in duplicate with 2 mL of complete growth medium (DMEM:F12 with 10% FBS) and the plates are incubated for about 24 hours at 37° C. with 5% CO₂. After the 24 hour incubation period, an aliquot of media from each well is removed and the amount of GLA protein secreted into the cell culture medium is quantitated by an enzymatic assay using a blue-fluorogenic substrate as described below in Example 1.

In an embodiment, the genetically modified cells are not islet cells, as defined herein. In an embodiment, the genetically modified cells have one or more of the following characteristics: (i) are not capable of producing insulin (e.g., insulin A-chain, insulin B-chain, or proinsulin) in an amount effective to treat diabetes or another disease or condition that may be treated with insulin; (ii) not capable of producing insulin in a glucose-responsive manner; or (iii) not derived from an induced pluripotent stem cell that was engineered or differentiated into insulin-producing pancreatic beta cells.

### Devices

A genetically modified cell described herein, e.g., a genetically modified RPE cell, or a plurality of such cells may be incorporated into, e.g., encapsulated within, an implantable device for use in providing a polypeptide encoded by the inserted transcription unit to a subject.

Exemplary implantable devices comprise materials such as metals, metallic alloys, ceramics, polymers, fibers, inert materials, and combinations thereof. The device can have any configuration and shape appropriate for supporting the viability and productivity of the encapsulated cells after implant into the intended target location. In some embodiments, the device is a hydrogel capsule, e.g., a millicapsule or a microcapsule (e.g., a hydrogel millicapsule or a hydrogel microcapsule). As non-limiting examples, device shapes may be cylinders, rectangles, disks, ovoids, stellates, or spheres. The device can be comprised of a mesh-like or nested structure. In some embodiments, a device is capable of preventing materials over a certain size from passing through a pore or opening. In some embodiments, a device (e.g., particle) is capable of preventing materials greater than 50 kD, 75 kD, 100 kD, 125 kD, 150 kD, 175 kD, 200 kD, 250 kD, 300 kD, 400 kD, 500 kD, 750 kD, or 1,000 kD from passing through.

The device (e.g., capsule, particle) may comprise (and optionally is configured to release) one or more exogenous agents that are not expressed by the engineered cells. Such exogenous agents may include, e.g., a nucleic acid (e.g., an RNA or DNA molecule), a protein (e.g., a hormone, an enzyme (e.g., glucose oxidase, kinase, phosphatase, oxygenase, hydrogenase, reductase), an antibody, an antibody fragment, an antigen, a small molecule, a lipid, a drug, vaccine, or any derivative thereof, a small-molecule, an active or inactive fragment of a protein or polypeptide. In some embodiments, the device comprises at least one means for mitigating the foreign body response (FBR), for example, mitigate the FBR when the device is implanted into or onto a subject.

The device can comprise a hydrogel capsule, or a plurality of the hydrogel capsule. The hydrogel capsule(s) in the device can have any of a variety of shapes: cylinder, cylinder with hemispherical ends (also known as spherocylinder), disc, noodle (e.g., as described in WO 2015/191547), sphere (e.g., as defined herein), or spheroid (e.g., as defined herein). In an embodiment, the hydrogel capsule(s) in a device are spheres, as defined herein.

In an embodiment, the device is a macroencapsulation device. Nonlimiting examples of macrodevices are described in: WO 2019/068059, WO 2019/169089, U.S. Pat. Nos. 9,526,880, 9,724,430 and 8,278,106; European Patent No. EP742818B1, and Sang, S. and Roy, S., Biotechnol. Bioeng. 113(7):1381-1402 (2016).

In an embodiment, the device is a macrodevice having one or more cell-containing compartments. A device with two or more cell-containing compartments may be configured to produce two or more proteins, e.g., cells expressing the ARSB fusion protein would be placed in one compartment and cells expressing a different protein (e.g., a therapeutic protein that can alleviate one or more symptoms of MPS-6) would be placed in a separate compartment. WO 2018/232027 describes a device with multiple cell-containing compartments formed in a micro-fabricated body and covered by a porous membrane.

In an embodiment, the device is configured as a thin, flexible strand as described in U.S. Pat. No. 10,493,107. This strand comprises a substrate, an inner polymeric coating surrounding the substrate and an outer hydrogel coating surrounding the inner polymeric coating. The protein-expressing cells are positioned in the outer coating.

A device described herein may be provided as a preparation or composition for implantation or administration to a subject, i.e., a device preparation or device composition. In some embodiments, a device preparation or device composition comprises at least 2, 4, 8, 16, 32, 64 or more devices, and at least 20%, 30%, 35%, 40%, 45%, 50%, 55%, 60%, 65%, 70%, 75%, 80%, 85%, 90%, 95% or 100% of the devices in the preparation or composition have a characteristic as described herein, e.g., mean capsule diameter, or number of cells in the cell-containing compartment.

In some embodiments, a device (e.g., particle) has a largest linear dimension (LLD), e.g., mean diameter, or size that is at least about 0.5 millimeter (mm), preferably about 1.0 mm, about 1.5 mm or greater. In some embodiments, a device can be as large as 10 mm in diameter or size.

For example, a device or particle described herein is in a size range of 0.5 mm to 10 mm, 1 mm to 10 mm, 1 mm to 8 mm, 1 mm to 6 mm, 1 mm to 5 mm, 1 mm to 4 mm, 1 mm to 3 mm, 1 mm to 2 mm, 1 mm to 1.5 mm, 1.5 mm to 8 mm, 1.5 mm to 6 mm, 1.5 mm to 5 mm, 1.5 mm to 4 mm, 1.5 mm to 3 mm, 1.5 mm to 2 mm, 2 mm to 8 mm, 2 mm to 7 mm, 2 mm to 6 mm, 2 mm to 5 mm, 2 mm to 4 mm, 2 mm to 3 mm, 2.5 mm to 8 mm, 2.5 mm to 7 mm, 2.5 mm to 6 mm, 2.5 mm to 5 mm, 2.5 mm to 4 mm, 2.5 mm to 3 mm, 3 mm to 8 mm, 3 mm to 7 mm, 3 mm to 6 mm, 3 mm to 5 mm, 3 mm to 4 mm, 3.5 mm to 8 mm, 3.5 mm to 7 mm, 3.5 mm to 6 mm, 3.5 mm to 5 mm, 3.5 mm to 4 mm, 4 mm to 8 mm, 4 mm to 7 mm, 4 mm to 6 mm, 4 mm to 5 mm, 4.5 mm to 8 mm, 4.5 mm to 7 mm, 4.5 mm to 6 mm, 4.5 mm to 5 mm, 5 mm to 8 mm, 5 mm to 7 mm, 5 mm to 6 mm, 5.5 mm to 8 mm, 5.5 mm to 7 mm, 5.5 mm to 6 mm, 6 mm to 8 mm, 6 mm to 7 mm, 6.5 mm to 8 mm, 6.5 mm to 7 mm, 7 mm to 8 mm, or 7.5 mm to 8 mm.

A device, device preparation or device composition may be configured for implantation, or is implanted or disposed, into or onto any site or part of the body. In some embodiments, the implantable device or device preparation is configured for implantation into the peritoneal cavity (e.g., the lesser sac, also known as the omental bursa or bursalis omentum). A device, device preparation or device composition may be implanted in the peritoneal cavity (e.g., the omentum, e.g., the lesser sac) or disposed on a surface within the peritoneal cavity (e.g., omentum, e.g., lesser sac) via injection or catheter. Additional considerations for implantation or disposition of a device, device preparation or device composition into the omentum (e.g., the lesser sac) are provided in M. Pellicciaro et al. (2017) CellR4 5(3): e2410.

In some embodiments, the implantable device comprises at least one cell-containing compartment comprising a plurality of live genetically modified cells encapsulated by a polymer composition. In an embodiment, the device contains two, three, four or more cell-containing compartments, each of which comprises a plurality of live, engineered cells described herein. In an embodiment, the cells in at least one of the compartments are capable of expressing and secreting an enzyme, e.g., a GLA protein or an IDUA protein, when the device is implanted into a subject.

In an embodiment, cells to be incorporated into a device described herein, e.g., a hydrogel capsule, are prepared in the form of a cell suspension prior to being encapsulated within the device. The cells in the suspension may take the form of single cells (e.g., from a monolayer cell culture), or provided in another form, e.g., disposed on a microcarrier (e.g., a bead or matrix) or as a three-dimensional aggregate of cells (e.g., a cell cluster or spheroid). The cell suspension can comprise multiple cell clusters (e.g., as spheroids) or microcarriers.

In some embodiments, the polymer composition in the cell-containing compartment(s) comprises a polysaccharide or other hydrogel-forming polymer (e.g., alginate, hyaluronate or chondroitin). In some embodiments, the polymer is an alginate, which is a polysaccharide made up of β-D-mannuronic acid (M) and α-L-guluronic acid (G). In some embodiments, the alginate has a low molecular weight (e.g., approximate molecular weight of <75 kD) and G:M ratio ≥1.5, (ii) a medium molecular weight alginate, e.g., has approximate molecular weight of 75-150 kDa and G:M ratio ≥1.5, (iii) a high molecular weight alginate, e.g., has an approximate MW of 150 kDa-250 kDa and G:M ratio ≥1.5, (iv) or a blend of two or more of these alginaes.

In some embodiments, the cell-containing compartment(s) further comprises at least one cell-binding substance (CBS), e.g., a cell-binding peptide (CBP) or cell-binding polypeptide (CBPP). In an embodiment, the CBS comprises a CBP covalently attached to polymer molecules in the polymer composition via a linker (“CBP-polymer”). In an embodiment, the polymer in the CBP-polymer is a polysaccharide (e.g., an alginate) or other hydrogel-forming polymer. Various cell-binding peptides for use in the devices of the disclosure are described herein. In an embodiment, the cell-binding peptide is 25 amino acids or less (e.g., 20, 15, 10 or less) in length and comprises the cell binding sequence of a ligand for a cell-adhesion molecule (CAM). In an embodiment, the cell-binding peptide consists essentially of a cell binding sequence shown in Table 1 of WO 2020/198685. In an embodiment, the cell binding sequence is RGD (SEQ ID NO: 34) or RGDSP (SEQ ID NO: 35). In an embodiment, the amino terminus of the cell-binding peptide is covalently attached to the polymer via an amino acid linker. In an embodiment, the amino acid linker consists essentially of one to three glycine residues. In an embodiment, the cell-binding peptide consists essentially of RGD (SEQ ID NO: 34) or RGDSP (SEQ ID NO: 35) and the linker consists essentially of a single glycine residue.

In some embodiments, the device further comprises at least one means for mitigating the foreign body response (FBR), for example, mitigates the FBR when the device is implanted into or onto a subject. Various means for mitigating the FBR of the devices are described herein, but any biological, chemical or physical element that is capable of reducing the FBR to the device compared to a reference device is contemplated herein.

For example, the means for mitigating the FBR in devices disclosed herein can comprise surrounding the cells with a semi-permeable biocompatible membrane having a pore size that is selected to allow oxygen and other molecules important to cell survival and function to move through the semi-permeable membrane while preventing immune cells from traversing through the pores. In an embodiment, the semi-permeable membrane has a molecular weight cutoff of less than 1000 kD or between 50-700 kD, 70-300 kD, or between 70-150 kD, or between 70 and 130 kD.

Another FBR-mitigating means comprises completely surrounding the cell-containing compartment with a barrier compartment formed from a cell-free biocompatible material, such as the two or three layer capsules described in WO 2014/153127, WO 2016/019391 or the core-shell microcapsules described in Ma, M et al., Adv. Healthc Mater., 2(5):667-672 (2012). Such a barrier compartment could be used with or without the semi-permeable membrane means. FBR-mitigating means can comprise disposing on or within the device an anti-inflammatory drug that is released from the implanted device to inhibit FBR, e.g., as described in U.S. Pat. No. 9,867,781. Other FBR-mitigating means employ a CSF-1R inhibitor that is disposed on the device surface or encapsulated within the device, as described in WO 2017/176792 and WO 2017/176804. Other FBR-mitigating means employ configuring the device in a spherical shape with a diameter of greater than 1 mm, as described in Veiseh, O., et al., Nature Materials 14:643-652 (2015).

In some embodiments, the means for mitigating the FBR comprises disposing an afibrotic compound or afibrotic polymer on the exterior surface of the device and/or within a barrier compartment surrounding the cell-containing compartment. In an embodiment, an afibrotic polymer comprises a biocompatible, zwitterionic polymer, e.g., as described in WO 2017/218507, WO 2018/140834, or Liu et al., Nature Communications (2019)10:5262. Exemplary afibrotic compounds include compounds of Formula (I) described herein below. In some embodiments, the device can comprise combinations of two or more of the above FBR-mitigating means.

In some embodiments, the device has two hydrogel compartments, in which the inner, cell-containing compartment is completely surrounded by the second, outer (e.g., barrier) compartment. In an embodiment, the inner boundary of the second compartment forms an interface with the outer boundary of the first compartment, e.g., as illustrated in FIG. 7.

In some embodiments, one or more compartments in a device comprises an afibrotic polymer, e.g., an afibrotic compound of Formula (I) (defined herein below) covalently attached to a polymer that is the same or different than the polymer in the CBP-polymer. In an embodiment, some or all the monomers in the afibrotic polymer are modified with the same compound of Formula (I). In some embodiments, some or all the monomers in the afibrotic polymer are modified with different compounds of Formula (I). In some embodiments in which the device is a two-compartment hydrogel capsule, the afibrotic polymer is present only in the outer, barrier compartment, including its outer surface.

The alginate in an afibrotic polymer can be chemically modified with a compound of Formula (I) using any suitable method known in the art. For example, the alginate carboxylic acid moiety can be activated for coupling to one or more amine-functionalized compounds to achieve an alginate modified with a compound of Formula (I). The alginate polymer may be dissolved in water (30 mL/gram polymer) and treated with an activating agent (e.g., 2-chloro-4,6-dimethoxy-1,3,5-triazine (0.5 eq)) and a base (e.g., N-methylmorpholine (1 eq)). To this mixture may be added a solution of the compound of Formula (I) in acetonitrile (0.3M). The reaction may be warmed to 55° C. for 16 h, then cooled to room temperature and gently concentrated via rotary evaporation, then the residue may be dissolved, e.g., in water. The mixture may then be filtered, e.g., through a bed of cyano-modified silica gel (Silicycle) and the filter cake washed with water. The resulting solution may then be dialyzed (10,000 MWCO membrane) against water for 24 hours, e.g., replacing the water twice. The resulting solution can be concentrated, e.g., via lyophilization, to afford the desired chemically modified alginate. The alginate in an afibrotic polymer can be chemically modified with a compound of Formula (I) using any suitable method known in the art, e.g., as described in any of WO 2019/195055, WO 2018/067615, WO 2017/075631, WO 2016/019391 and WO 2012/167223.

One or more compartments in a device may comprise an unmodified polymer that is the same or different than the polymer in the CBP-polymer and in any afibrotic polymer that is present in the device. In an embodiment, the first compartment, second compartment or all compartments in the device comprises the unmodified polymer. In some embodiments, the unmodified polymer is an unmodified alginate. In an embodiment, the unmodified alginate has a molecular weight of 150 kDa-250 kDa and a G:M ratio of ≥1.5.

### Compounds of Formula (I)

In some embodiments, the devices described herein comprise a compound of Formula (I):

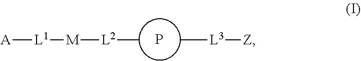

or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₂-C₆-alkenylene)-, —N(R^{C})N(R^{D})—, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})—, —Si(OR^{A})₂—, —Si(R^{G})(OR^{A}), —B(OR^{A}), or a metal, each of which is optionally linked to an attachment group (e.g., an attachment group described herein) and is optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³;
P is absent, cycloalkyl, heterocycyl, or heteroaryl, each of which is optionally substituted by one or more R⁴;
Z is hydrogen, alkyl, alkenyl, alkynyl, heteroalkyl, —OR^{A}, —C(O)R^{A}, —C(O)OR^{A}, —C(O)N(R^{C})(R^{D}), —N(R^{C})C(O)R^{A}, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

In some embodiments, the compound of Formula (I) is a compound of Formula (I-a):

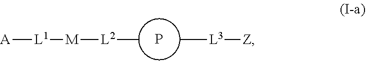

or a salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})N(R^{D})—, —NCN—, —N(R^{C})C(O)(C₁-C₆-alkylene)-, —N(R^{C})C(O)(C₂-C₆-alkenylene)-, —C(═N(R^{C})(R^{D}))O—, —S—, —S(O), —OS(O)ₓ, —N(R^{C})S(O)ₓ, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³;
P is heteroaryl optionally substituted by one or more R⁴;
Z is alkyl, alkenyl, alkynyl, heteroalkyl, cycloalkyl, heterocyclyl, aryl, or heteroaryl, each of which is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y} cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

In some embodiments, for Formulas (I) or (I-a), A is alkyl, alkenyl, alkynyl, heteroalkyl, cycloalkyl, heterocyclyl, aryl, heteroaryl, —O—, —C(O)O—, —C(O)—, —OC(O)—, —N(R^{C})C(O)—, —N(R^{C})C(O)(C₁-C₆-alkylene)-, —N(R^{C})C(O)(C₂-C₆-alkenylene)-, or —N(R^{C})—. In some embodiments, A is alkyl, alkenyl, alkynyl, heteroalkyl, cycloalkyl, heterocyclyl, aryl, heteroaryl, —O—, —C(O)O—, —C(O)—, —OC(O)—, or —N(R^{C})—. In some embodiments, A is alkyl, alkenyl, alkynyl, heteroalkyl, —O—, —C(O)O—, —C(O)—, —OC(O)—, or —N(R^{C})—. In some embodiments, A is alkyl, —O—, —C(O)O—, —C(O)—, —OC(O), or —N(R^{C})—. In some embodiments, A is —N(R^{C})C(O)—, —N(R^{C})C(O)(C₁-C₆-alkylene)-, or —N(R^{C})C(O)(C₁-C₆-alkenylene)-. In some embodiments, A is —N(R^{C})—. In some embodiments, A is —N(R^{C})— and R^{C} is independently hydrogen or alkyl. In some embodiments, A is —NH—. In some embodiments, A is —N(R^{C})C(O)(C₁-C₆-alkylene)-, wherein alkylene is substituted with R¹. In some embodiments, A is —N(R^{C})C(O)(C₁-C₆-alkylene)-, and R¹ is alkyl (e.g., methyl). In some embodiments, A is —NHC(O)C(CH₃)₂—. In some embodiments, A is —N(R^{C})C(O)(methylene)-, and R¹ is alkyl (e.g., methyl). In some embodiments, A is —NHC(O)CH(CH₃)—. In some embodiments, A is —NHC(O)C(CH₃)—.

In some embodiments, for Formulas (I) or (I-a), L¹ is a bond, alkyl, or heteroalkyl. In some embodiments, L¹ is a bond or alkyl. In some embodiments, L¹ is a bond. In some embodiments, L¹ is alkyl. In some embodiments, L¹ is C₁-C₆ alkyl. In some embodiments, L¹ is —CH₂—, —CH(CH₃)—, —CH₂CH₂CH₂, or —CH₂CH₂—. In some embodiments, L¹ is —CH₂— or —CH₂CH₂—.

In some embodiments, for Formulas (I) or (I-a), L³ is a bond, alkyl, or heteroalkyl. In some embodiments, L³ is a bond. In some embodiments, L³ is alkyl. In some embodiments, L³ is C₁-C₁₂ alkyl. In some embodiments, L³ is C₁-C₆ alkyl. In some embodiments, L³ is —CH₂—. In some embodiments, L³ is heteroalkyl. In some embodiments, L³ is C₁-C₁₂ heteroalkyl, optionally substituted with one or more R² (e.g., oxo). In some embodiments, L³ is C₁-C₆ heteroalkyl, optionally substituted with one or more R² (e.g., oxo). In some embodiments, L³ is —C(O)OCH₂—, —CH₂(OCH₂CH₂)₂—, —CH₂(OCH₂CH₂)₃—, CH₂CH₂O—, or —CH₂O—. In some embodiments, L³ is —CH₂O—.

In some embodiments, for Formulas (I) or (I-a), M is absent, alkyl, heteroalkyl, aryl, or heteroaryl. In some embodiments, M is heteroalkyl, aryl, or heteroaryl. In some embodiments, M is absent. In some embodiments, M is alkyl (e.g., C₁-C₆ alkyl). In some embodiments, M is —CH₂—. In some embodiments, M is heteroalkyl (e.g., C₁-C₆ heteroalkyl). In some embodiments, M is (—OCH₂CH₂-)z, wherein z is an integer selected from 1 to 10. In some embodiments, z is an integer selected from 1 to 5. In some embodiments, M is —OCH₂CH₂—, (—OCH₂CH₂—)₂, (—OCH₂CH₂—)₃, (—OCH₂CH₂—)₄, or (—OCH₂CH₂—)₅. In some embodiments, M is —OCH₂CH₂—, (—OCH₂CH₂—)₂, (—OCH₂CH₂—)₃, or (—OCH₂CH₂—)₄. In some embodiments, M is (—OCH₂CH₂—)₃.

In some embodiments, M is aryl. In some embodiments, M is phenyl. In some embodiments, M is unsubstituted phenyl. In some embodiments, M is

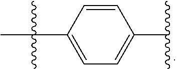 [SMILES: *c1ccc(*)cc1]

In some embodiments, M is phenyl substituted with R⁷ (e.g., 1 R⁷). In some embodiments, M is

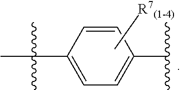

In some embodiments, R⁷ is CF₃.

In some embodiments, for Formulas (I) or (I-a), P is absent, heterocyclyl, or heteroaryl. In some embodiments, P is absent. In some embodiments, for Formulas (I) and (I-a), P is a tricyclic, bicyclic, or monocyclic heteroaryl. In some embodiments, P is a monocyclic heteroaryl. In some embodiments, P is a nitrogen-containing heteroaryl. In some embodiments, P is a monocyclic, nitrogen-containing heteroaryl. In some embodiments, P is a 5-membered heteroaryl. In some embodiments, P is a 5-membered nitrogen-containing heteroaryl. In some embodiments, P is tetrazolyl, imidazolyl, pyrazolyl, or triazolyl, pyrrolyl, oxazolyl, or thiazolyl. In some embodiments, P is tetrazolyl, imidazolyl, pyrazolyl, or triazolyl, or pyrrolyl. In some embodiments, P is imidazolyl. In some embodiments, P is

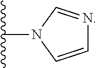 [SMILES: *n1ccnc1]

In some embodiments, P is triazolyl. In some embodiments, P is 1,2,3-triazolyl optionally substituted with R⁴. In some embodiments, P is

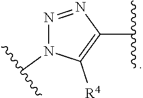

In some embodiments, P is 1,2,3-triazolyl. In some embodiments, P is

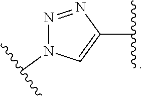 [SMILES: *c1cn(*)nn1]

In some embodiments, P is heterocyclyl. In some embodiments, P is a 5-membered heterocyclyl or a 6-membered heterocyclyl. In some embodiments, P is imidazolidinonyl. In some embodiments, P is

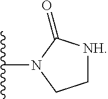 [SMILES: *N1CCNC1=O]

In some embodiments, P is thiomorpholinyl-1,1-dioxidyl.

In some embodiments, P is

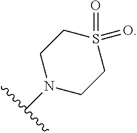 [SMILES: *N1CCS(=O)(=O)CC1]

In some embodiments, for Formulas (I) or (I-a), Z is alkyl, heteroalkyl, cycloalkyl, heterocyclyl, aryl, or heteroaryl. In some embodiments, Z is heterocyclyl. In some embodiments, Z is monocyclic or bicyclic heterocyclyl. In some embodiments, Z is an oxygen-containing heterocyclyl. In some embodiments, Z is a 4-membered heterocyclyl, 5-membered heterocyclyl, or 6-membered heterocyclyl. In some embodiments, Z is a 6-membered heterocyclyl. In some embodiments, Z is a 6-membered oxygen-containing heterocyclyl. In some embodiments, Z is tetrahydropyranyl. In some embodiments, Z is

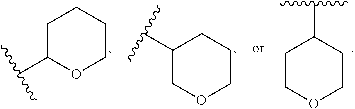

In some embodiments, Z is a 4-membered oxygen-containing heterocyclyl. In some embodiments, Z is

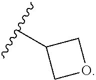 [SMILES: *C1COC1]

In some embodiments, Z is a bicyclic oxygen-containing heterocyclyl. In some embodiments, Z is phthalic anhydridyl. In some embodiments, Z is a sulfur-containing heterocyclyl. In some embodiments, Z is a 6-membered sulfur-containing heterocyclyl. In some embodiments, Z is a 6-membered heterocyclyl containing a nitrogen atom and a sulfur atom. In some embodiments, Z is thiomorpholinyl-1,1-dioxidyl. In some embodiments, Z is

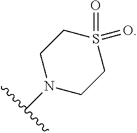 [SMILES: *N1CCS(=O)(=O)CC1]

In some embodiments, Z is a nitrogen-containing heterocyclyl. In some embodiments, Z is a 6-membered nitrogen-containing heterocyclyl. In some embodiments, Z is

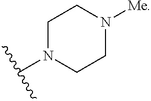 [SMILES: *N1CCN(C)CC1]

In some embodiments, Z is a bicyclic heterocyclyl. In some embodiments, Z is a bicyclic nitrogen-containing heterocyclyl, optionally substituted with one or more R⁵. In some embodiments, Z is 2-oxa-7-azaspiro[3.5]nonanyl. In some embodiments, Z is

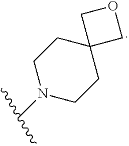 [SMILES: *N1CCC2(CC1)COC2]

In some embodiments, Z is 1-oxa-3,8-diazaspiro[4.5]decan-2-one. In some embodiments, Z is

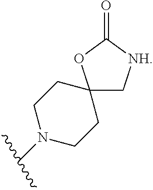 [SMILES: *N1CCC2(CC1)CNC(=O)O2]

In some embodiments, for Formulas (I) or (I-a), Z is aryl. In some embodiments, Z is monocyclic aryl. In some embodiments, Z is phenyl. In some embodiments, Z is monosubstituted phenyl (e.g., with 1 R⁵). In some embodiments, Z is monosubstituted phenyl, wherein the 1 R⁵ is a nitrogen-containing group. In some embodiments, Z is monosubstituted phenyl, wherein the 1 R⁵ is NH₂. In some embodiments, Z is monosubstituted phenyl, wherein the 1 R⁵ is an oxygen-containing group. In some embodiments, Z is monosubstituted phenyl, wherein the 1 R⁵ is an oxygen-containing heteroalkyl. In some embodiments, Z is monosubstituted phenyl, wherein the 1 R⁵ is OCH₃. In some embodiments, Z is monosubstituted phenyl, wherein the 1 R⁵ is in the ortho position. In some embodiments, Z is monosubstituted phenyl, wherein the 1 R⁵ is in the meta position. In some embodiments, Z is monosubstituted phenyl, wherein the 1 R⁵ is in the para position.

In some embodiments, for Formulas (I) or (I-a), Z is alkyl. In some embodiments, Z is C₁-C₁₂ alkyl. In some embodiments, Z is C₁-C₁₀ alkyl. In some embodiments, Z is C₁-C₅ alkyl. In some embodiments, Z is C₁-C₅ alkyl substituted with 1-5 R⁵. In some embodiments, Z is C₁-C₈ alkyl substituted with 1 R⁵. In some embodiments, Z is C₁-C₈ alkyl substituted with 1 R⁵, wherein R⁵ is alkyl, heteroalkyl, halogen, oxo, —OR^{A1}, —C(O)OR^{A1}, —C(O)R^{B1}, —OC(O)R^{B1}, or —N(R^{C1})(R^{D1}). In some embodiments, Z is C₁-C₈ alkyl substituted with 1 R⁵, wherein R⁵ is —OR^{A1} or —C(O)OR^{A1}. In some embodiments, Z is C₁-C₈ alkyl substituted with 1 R⁵, wherein R⁵ is —OR^{A1} or —C(O)OH. In some embodiments, Z is —CH₃.

In some embodiments, for Formulas (I) or (I-a), Z is heteroalkyl. In some embodiments, Z is C₁-C₁₂ heteroalkyl. In some embodiments, Z is C₁-C₁₀ heteroalkyl. In some embodiments, Z is C₁-C₈ heteroalkyl. In some embodiments, Z is C₁-C₆ heteroalkyl. In some embodiments, Z is a nitrogen-containing heteroalkyl optionally substituted with one or more R⁵. In some embodiments, Z is a nitrogen and sulfur-containing heteroalkyl substituted with 1-5 R⁵. In some embodiments, Z is N-methyl-2-(methylsulfonyl)ethan-1-aminyl.

In some embodiments, Z is —OR^{A} or —C(O)OR^{A}. In some embodiments, Z is —OR^{A} (e.g., —OH or —OCH₃). In some embodiments, Z is —OCH₃. In some embodiments, Z is —C(O)OR^{A} (e.g., —C(O)OH).

In some embodiments, Z is hydrogen.

In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound of Formula (I) is a compound of Formula (I-b):

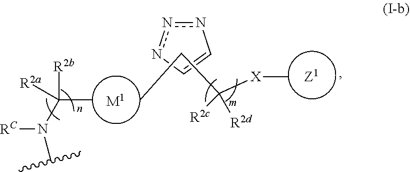

or a salt thereof, wherein Ring M¹ is cycloalkyl, heterocyclyl, aryl, or heteroaryl, each of which is optionally substituted with 1-5 R³; Ring Z¹ is cycloalkyl, heterocyclyl, aryl or heteroaryl, optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl, or each of R^{2a} and R^{2b} or R^{2c} and R^{2d} is taken together to form an oxo group; X is absent, N(R¹⁰), O, or S; R^{C} is hydrogen, alkyl, alkenyl, alkynyl, heteroalkyl, cycloalkyl, heterocyclyl, aryl, or heteroaryl, wherein each of alkyl, alkenyl, alkynyl, heteroalkyl, cycloalkyl, heterocyclyl, aryl, or heteroaryl is optionally substituted with 1-6 R⁶; each R³, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D}1), —N(R^{C1})C(O)R^{B1}, —C(O)N(R^{C1}), SR^{E1} cycloalkyl, heterocyclyl, aryl, or heteroaryl; R¹⁰ is hydrogen, alkyl, alkenyl, alkynyl, heteroalkyl, —C(O)OR^{A1}, —C(O)R^{B1}, —OC(O)R^{B1}, —C(O)N(R^{C1}), cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; each R⁷ is independently alkyl, alkenyl, alkynyl, heteroalkyl, halogen, cyano, oxo, hydroxyl, cycloalkyl, or heterocyclyl; each m and n is independently 1, 2, 3, 4, 5, or 6; and “ [symbol] ” refers to a connection to an attachment group or a polymer described herein. In some embodiments, for each R³ and R⁵, each alkyl, alkenyl, alkynyl, heteroalkyl, cycloalkyl, heterocyclyl, aryl, or heteroaryl is optionally and independently substituted with halogen, oxo, cyano, cycloalkyl, or heterocyclyl.

In some embodiments, the compound of Formula (I-b) is a compound of Formula (I-b-i):

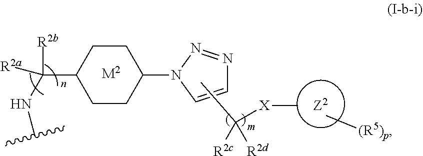

or a pharmaceutically acceptable salt thereof, wherein Ring M² is aryl or heteroaryl optionally substituted with one or more R³; Ring Z² is cycloalkyl, heterocyclyl, aryl, or heteroaryl; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; X is absent, O, or S; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; or two R⁵ are taken together to form a 5-6 membered ring fused to Ring Z²; each R^{A1} and R^{B1} is independently hydrogen, alkyl, or heteroalkyl; m and n are each independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I-b-i) is a compound of Formula (I-b-ii):

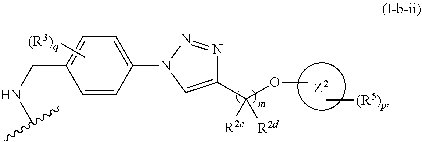

or a pharmaceutically acceptable salt thereof, wherein Ring Z² is cycloalkyl, heterocyclyl, aryl or heteroaryl; each of R^{2c} and R^{2d} is independently hydrogen, alkyl, or heteroalkyl, or R^{2c} and R^{2d} and taken together to form an oxo group; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; each of p and q is independently 0, 1, 2, 3, 4, 5, or 6; and “ [symbol] ” refers to a connection to an attachment group or a polymer described herein.

In some embodiments, the compound of Formula (I) is a compound of Formula (I-c):

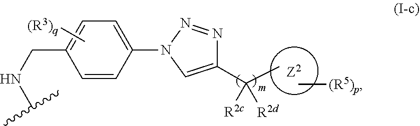

or a pharmaceutically acceptable salt thereof, wherein Ring Z² is cycloalkyl, heterocyclyl, aryl or heteroaryl; each of R^{2c} and R^{2d} is independently hydrogen, alkyl, or heteroalkyl, or R^{2c} and R^{2d} is taken together to form an oxo group; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; m is 0, 1, 2, 3, 4, 5, or 6; each of p and q is independently 0, 1, 2, 3, 4, 5, or 6; and “ [symbol] ” refers to a connection to an attachment group or a polymer described herein.

In some embodiments, the compound of Formula (I) is a compound of Formula (I-c-i):

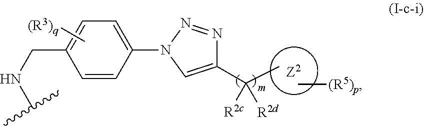

or a pharmaceutically acceptable salt thereof, wherein Ring Z² is cycloalkyl, heterocyclyl, aryl or heteroaryl; each of R^{2c} and R^{2d} is independently hydrogen, alkyl, or heteroalkyl, or R^{2c} and R^{2d} is taken together to form an oxo group; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; m is 1, 2, 3, 4, 5, or 6; each of p and q is independently 0, 1, 2, 3, 4, 5, or 6; and “ [symbol] ” refers to a connection to an attachment group or a polymer described herein.

In some embodiments, the compound of Formula (I) is a compound of Formula (I-d):

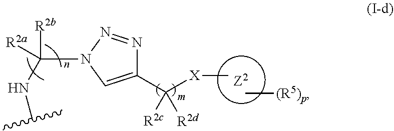

or a pharmaceutically acceptable salt thereof, wherein Ring Z² is cycloalkyl, heterocyclyl, aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I) is a compound of Formula (I-e):

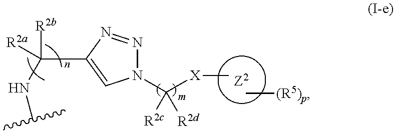

or a pharmaceutically acceptable salt thereof, wherein Ring Z² is cycloalkyl, heterocyclyl, aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I) is a compound of Formula (I-f):

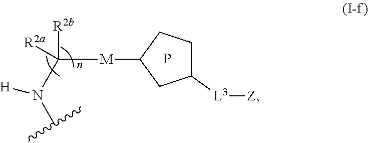

or a pharmaceutically acceptable salt thereof, wherein M is alkyl optionally substituted with one or more R³; Ring P is heteroaryl optionally substituted with one or more R⁴; L³ is alkyl or heteroalkyl optionally substituted with one or more R²; Z is alkyl, heteroalkyl, cycloalkyl, heterocyclyl, aryl, or heteroaryl, each of which is optionally substituted with one or more R⁵; each of R^{2a} and R^{2b} is independently hydrogen, alkyl, or heteroalkyl, or R^{2a} and R^{2b} is taken together to form an oxo group; each R², R³, R⁴, and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (I) is a compound of Formula (II):

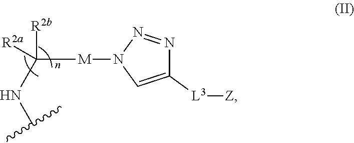

or a pharmaceutically acceptable salt thereof, wherein M is a bond, alkyl or aryl, wherein alkyl and aryl is optionally substituted with one or more R³; L³ is alkyl or heteroalkyl optionally substituted with one or more R²; Z is hydrogen, alkyl, heteroalkyl, cycloalkyl, heterocyclyl, aryl, heteroaryl or —OR^{A}, wherein alkyl, heteroalkyl, cycloalkyl, heterocyclyl, aryl, and heteroaryl is optionally substituted with one or more R⁵; R^{A} is hydrogen; each of R^{2a} and R^{2b} is independently hydrogen, alkyl, or heteroalkyl, or R^{2a} and R^{2b} is taken together to form an oxo group; each R², R³, and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (II) is a compound of Formula (II-a):

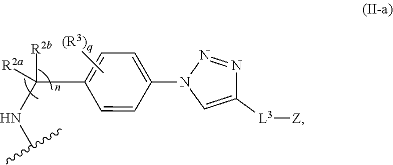

or a pharmaceutically acceptable salt thereof, wherein L³ is alkyl or heteroalkyl, each of which is optionally substituted with one or more R²; Z is hydrogen, alkyl, heteroalkyl, or —OR^{A}, wherein alkyl and heteroalkyl are optionally substituted with one or more R⁵; each of R^{2a} and R^{2b} is independently hydrogen, alkyl, or heteroalkyl, or R^{2a} and R^{2b} is taken together to form an oxo group; each R², R³, and R⁵ is independently alkyl, heteroalkyl, halogen, oxo, —OR^{A1}, —C(O)OR^{A1}, or —C(O)R^{B1}; R^{A} is hydrogen; each R^{A1} and R^{B1} is independently hydrogen, alkyl, or heteroalkyl; n is independently 1, 2, 3, 4, 5, or 6; q is independently 1, 2, 3, or 4; and “ [symbol] ” refers to a connection to an attachment group or a polymer described herein.

In some embodiments, the compound of Formula (I) is a compound of Formula (III):

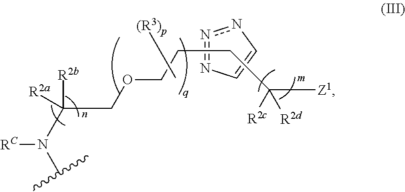

or a pharmaceutically acceptable salt thereof, wherein Z¹ is alkyl, alkenyl, alkynyl, heteroalkyl, cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl; or R^{2a} and R^{2b} or R^{2c} and R^{2d} are taken together to form an oxo group; R^{C} is hydrogen, alkyl, alkenyl, alkynyl, or heteroalkyl, wherein each of alkyl, alkenyl, alkynyl, or heteroalkyl is optionally substituted with 1-6 R⁶; each of R³, R⁵, and R⁶ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; q is an integer from 0 to 25; p is an integer from 0 to 4; and “ [symbol] ” refers to a connection to an attachment group or a polymer described herein.

In some embodiments, the compound of Formula (III) is a compound of Formula (III-a):

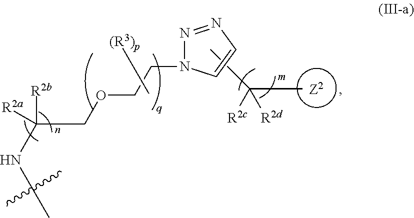

or a pharmaceutically acceptable salt thereof, wherein Ring Z² is cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; q is an integer from 0 to 25; p is an integer from 0 to 4; and “ [symbol] ” refers to a connection to an attachment group or a polymer described herein.

In some embodiments, the compound of Formula (III-a) is a compound of Formula (III-b):

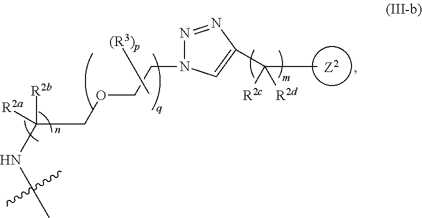

or a pharmaceutically acceptable salt thereof, wherein Ring Z² is cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; q is an integer from 0 to 25; p is an integer from 0 to 4; and “ [symbol] ” refers to a connection to an attachment group or a polymer described herein.

In some embodiments, the compound of Formula (III-a) is a compound of Formula (III-c):

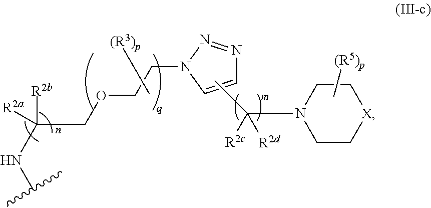

or a pharmaceutically acceptable salt thereof, wherein X is C(R′)(R″), N(R′), or S(O)ₓ; each of R′ and R″ is independently hydrogen, alkyl, halogen, or cycloalkyl; each of R^{2a}, R^{2b}, R^{2c}, and R^{2d} is independently hydrogen, alkyl, heteroalkyl, or halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; q is an integer from 0 to 25; p is an integer from 0 to 4; x is 0, 1, or 2; and “ [symbol] ” refers to a connection to an attachment group or a polymer described herein.

In some embodiments, the compound of Formula (III-c) is a compound of Formula (III-d):

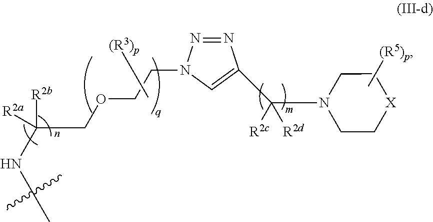

or a pharmaceutically acceptable salt thereof, wherein X is C(R′)(R″), N(R′), or S(O)ₓ; each of R′ and R″ is independently hydrogen, alkyl, halogen, or cycloalkyl; each of R^{2a}, R^{2b}, R^{2c}, and R^{2d} is independently hydrogen, alkyl, heteroalkyl, or halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; p is 0, 1, 2, 3, or 4; q is an integer from 0 to 25; and “ [symbol] ” refers to a connection to an attachment group or a polymer described herein.

In some embodiments, the compound is a compound of Formula (I). In some embodiments, L² is a bond and P and L³ are independently absent.

In some embodiments, the compound of Formula (I) is a compound of Formula (III-e):

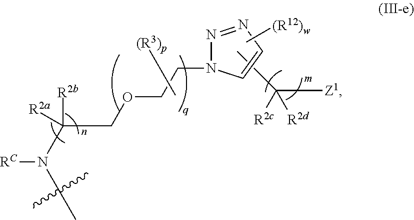

or a pharmaceutically acceptable salt thereof, wherein Z¹ is alkyl, alkenyl, alkynyl, heteroalkyl, cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl; or each of R^{2a} and R^{2b} or R^{2c} and R^{2d} is taken together to form an oxo group; R^{C} is hydrogen, alkyl, alkenyl, alkynyl, or heteroalkyl, wherein each of alkyl, alkenyl, alkynyl, or heteroalkyl is optionally substituted with 1-6 R⁶; each of R³, R⁵, and R⁶ is independently alkyl, heteroalkyl, halogen, oxo, —OR^{A1}, —C(O)OR^{A1}, or —C(O)R^{B1}; each R¹² is independently deuterium, alkyl, heteroalkyl, haloalkyl, halo, cyano, nitro, or amino; each R^{A1} and R^{B1} is independently hydrogen, alkyl, or heteroalkyl; m and n are each independently 1, 2, 3, 4, 5, or 6; q is an integer from 0 to 25; w is 0 or 1; and “ [symbol] ” refers to a connection to an attachment group or a polymer described herein.

In some embodiments, the compound of Formula (I) is a compound of Formula (III-f):

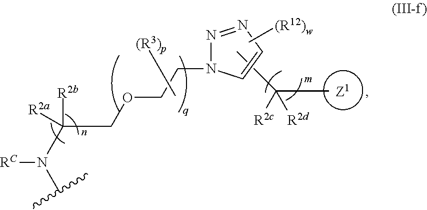

or a pharmaceutically acceptable salt thereof, wherein Ring Z¹ is cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; R^{C} is hydrogen, alkyl, alkenyl, alkynyl, or heteroalkyl, wherein each of alkyl, alkenyl, alkynyl, or heteroalkyl is optionally substituted with 1-6 R⁶; each of R³, R⁵, and R⁶ is independently alkyl, heteroalkyl, halogen, oxo, —OR^{A1}, —C(O)OR^{A1}, or —C(O)R^{B1}; each R¹² is independently deuterium, alkyl, heteroalkyl, haloalkyl, halo, cyano, nitro, or amino; each R^{A1} and R^{B1} is independently hydrogen, alkyl, or heteroalkyl; m and n are each independently 1, 2, 3, 4, 5, or 6; o and p are each independently 0, 1, 2, 3, 4, or 5; q is an integer from 0 to 25; w is 0 or 1; and “ [symbol] ” refers to a connection to an attachment group or a polymer described herein.

In some embodiments, the compound of Formula (I) is a compound of Formula (III-g):

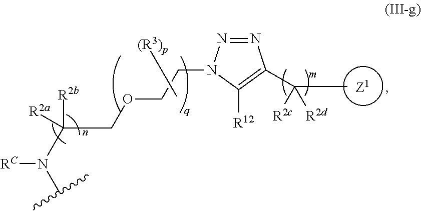

or a pharmaceutically acceptable salt thereof, wherein Ring Z¹ is cycloalkyl, heterocyclyl, aryl, or heteroaryl, each of which is optionally substituted with 1-5 R⁵; R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R³, R⁵, and R⁶ is independently alkyl, heteroalkyl, halogen, oxo, —OR^{A1}, —C(O)OR^{A1}, or —C(O)R^{B1}; R¹² is hydrogen, deuterium, alkyl, heteroalkyl, haloalkyl, halo, cyano, nitro, or amino; each R^{A1} and R^{B1} is independently hydrogen, alkyl, or heteroalkyl; m and n are each independently 1, 2, 3, 4, 5, or 6; q is an integer from 0 to 25; x is 0, 1, or 2; and “ [symbol] ” refers to a connection to an attachment group or a polymer described herein.

In some embodiments, the compound of Formula (I) is a compound of Formula (III-h):

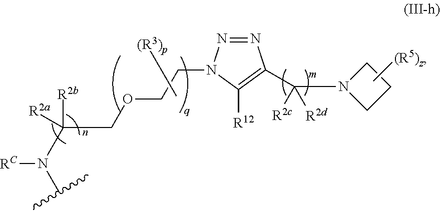

or a pharmaceutically acceptable salt thereof, wherein R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R³, R⁵, and R⁶ is independently alkyl, heteroalkyl, halogen, oxo, —OR^{A1}, —C(O)OR^{A1}, or —C(O)R^{B1}; R¹² is hydrogen, deuterium, alkyl, heteroalkyl, haloalkyl, halo, cyano, nitro, or amino; each R^{A1} and R^{B1} is independently hydrogen, alkyl, or heteroalkyl; m and n are each independently 1, 2, 3, 4, 5, or 6; q is an integer from 0 to 25; x is 0, 1, or 2; z is 0, 1, 2, 3, 4, 5, or 6, and “ [symbol] ” refers to a connection to an attachment group or a polymer described herein.

In some embodiments, the compound of Formula (I) is a compound of Formula (III-i):

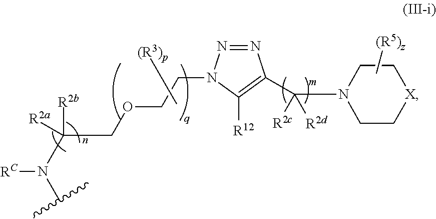

or a pharmaceutically acceptable salt thereof, wherein X is C(R′)(R″), N(R′), or S(O)ₓ; each of R′ and R″ is independently hydrogen, alkyl, or halogen; R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R³, R⁵, and R⁶ is independently alkyl, heteroalkyl, halogen, oxo, —OR^{A1}, —C(O)OR^{A1}, or —C(O)R^{B1}; R¹² is hydrogen, deuterium, alkyl, heteroalkyl, haloalkyl, halo, cyano, nitro, or amino; each R^{A1} and R^{B1} is independently hydrogen, alkyl, or heteroalkyl; m and n are each independently 1, 2, 3, 4, 5, or 6; q is an integer from 0 to 25; x is 0, 1, or 2; z is 0, 1, 2, 3, 4, 5, or 6, and “ [symbol] ” refers to a connection to an attachment group or a polymer described herein.

In some embodiments, X is S(O)ₓ. In some embodiments, x is 2. In some embodiments, X is S(O)₂.

In some embodiments, each of R^{2a}, R^{2b}, R^{2c}, and R^{2d} is independently hydrogen.

In some embodiments, R^{C} is hydrogen, —C(O)(C₁-C₆-alkyl), or —C(O)(C₁-C₆-alkenyl). In some embodiments, each of alkyl and alkenyl is substituted with 1 R⁶ (e.g., —CH₃). In some embodiments, R^{C} is hydrogen.

In some embodiments, n is 1. In some embodiments, q is 2, 3, 4, or 5. In some embodiments, q is 3. In some embodiments, m is 1. In some embodiments, p is 0. In some embodiments, R¹² is halo (e.g., Cl).

In some embodiments, the compound is a compound of Formula (I). In some embodiments, L² is a bond and P and L³ are independently absent.

In some embodiments, the compound is a compound of Formula (I-a). In some embodiments of Formula (II-a), L² is a bond, P is heteroaryl, L³ is a bond, and Z is hydrogen. In some embodiments, P is heteroaryl, L³ is heteroalkyl, and Z is alkyl. In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound is a compound of Formula (III-c). In some embodiments of Formula (III-c), each of R^{2a}, R^{2b}, R^{2c}, R^{2d}, R^{C} and R¹² is independently hydrogen, m is 1, n is 2, q is 3, p is 0, z is 1, and R⁵ is (e.g., —S(O)₂(CH₃)). In some embodiments, the compound of Formula (III-c) is Compound 123.

In some embodiments, the compound is a compound of Formula (I-a). In some embodiments of Formula (I-a), L² is a bond, P is heteroaryl, L³ is a bond, and Z is hydrogen. In some embodiments of Formula (I-a), P is heteroaryl, L³ is heteroalkyl, and Z is alkyl. In some embodiments of Formula (I-a), L² is a bond and P and L³ are independently absent. In some embodiments of Formula (I-a), L² is a bond, P is heteroaryl, L³ is a bond, and Z is hydrogen. In some embodiments of Formula (I-a), P is heteroaryl, L³ is heteroalkyl, and Z is alkyl.

In some embodiments, the compound is a compound of Formula (I-a). In some embodiments, A is absent; P is absent, L¹ is —NHCH₂, L² is a bond, M is aryl (e.g., phenyl), L³ is a bond, and Z is heterocyclyl (e.g., a nitrogen-containing heterocyclyl, e.g., thiomorpholinyl-1,1-dioxide). In some embodiments, the compound of Formula (I-a) is Compound 116.

In some embodiments of Formula (I-a), A is absent; P is absent, L¹ is —NHCH₂, L² is a bond, M is aryl (e.g., phenyl), L³ is CH₂O, and Z is heterocyclyl (e.g., an oxygen-containing heterocyclyl, e.g., tetrahydropyranyl, tetrahydrofuranyl, oxetanyl, or oxiranyl). In some embodiments, the compound of Formula (I-b) is Compound 105.

In some embodiments, the compound is a compound of Formula (I-b-i). In some embodiments of Formula (I-b-i), each of R^{2a} and R^{2b} is independently hydrogen or CH₃, each of R^{2c} and R^{2d} is independently hydrogen, m is 1 or 2, n is 1, X is O, p is 0, M² is phenyl optionally substituted with one or more R³, R³ is —CF₃, and Z² is heterocyclyl (e.g., an oxygen-containing heterocyclyl, e.g., tetrahydropyranyl, tetrahydrofuranyl, oxetanyl, or oxiranyl). In some embodiments, the compound of Formula (I-b-i) is Compound 100, Compound 106, Compound 107, Compound 108, Compound 109, or Compound 111.

In some embodiments, the compound is a compound of Formula (I-b-ii). In some embodiments of Formula (I-b-ii), each of R^{2c}, and R^{2d} is independently hydrogen, q is 0, p is 0, m is 1, and Z² is heterocyclyl (e.g., an oxygen-containing heterocyclyl, e.g., tetrahydropyranyl). In some embodiments, the compound of Formula (I-b-ii) is Compound 100.

In some embodiments, the compound is a compound of Formula (I-c). In some embodiments of Formula (I-c), each of R^{2c} and R^{2d} is independently hydrogen, m is 1, p is 1, q is 0, R⁵ is —CH₃, and Z is heterocyclyl (e.g., a nitrogen-containing heterocyclyl, e.g., piperazinyl). In some embodiments, the compound of Formula (I-c) is Compound 113.

In some embodiments, the compound is a compound of Formula (I-d). In some embodiments of Formula (I-d), each of R^{2a}, R^{2b}, R^{2c}, and R^{2d} is independently hydrogen, m is 1, n is 3, X is O, p is 0, and Z is heterocyclyl (e.g., an oxygen-containing heterocyclyl, e.g., tetrahydropyranyl, tetrahydrofuranyl, oxetanyl, or oxiranyl). In some embodiments, the compound of Formula (I-d) is Compound 110 or Compound 114.

In some embodiments, the compound is a compound of Formula (I-f). In some embodiments of Formula (I-f), each of R^{2a} and R^{2b} is independently hydrogen, n is 1, M is —CH₂—, P is a nitrogen-containing heteroaryl (e.g., imidazolyl), L³ is —C(O)OCH₂—, and Z is —CH₃. In some embodiments, the compound of Formula (I-f) is Compound 115.

In some embodiments, the compound is a compound of Formula (II-a). In some embodiments of Formula (II-a), each of R^{2a} and R^{2b} is independently hydrogen, n is 1, q is 0, L³ is —CH₂(OCH₂CH₂)₂, and Z is —OCH₃. In some embodiments, the compound of Formula (II-a) is Compound 112.

In some embodiments of Formula (II-a), each of R^{2a} and R^{2b} is independently hydrogen, n is 1, L³ is a bond or —CH₂, and Z is hydrogen or —OH. In some embodiments, the compound of Formula (II-a) is Compound 103 or Compound 104.

In some embodiments, the compound is a compound of Formula (III). In some embodiments of Formula (III), each of R^{2a}, R^{2b}, R^{2c}, and R^{2d} is independently hydrogen, m is 1, n is 2, q is 3, p is 0, R^{C} is hydrogen, and Z¹ is heteroalkyl optionally substituted with R⁵ (e.g., —N(CH₃)(CH₂CH₂)S(O)₂CH₃). In some embodiments, the compound of Formula (III) is Compound 120.

In some embodiments, the compound is a compound of Formula (III-b). In some embodiments of Formula (III-b), each of R^{2a}, R^{2b}, R^{2c}, and R^{2d} is independently hydrogen, m is 0, n is 2, q is 3, p is 0, and Z² is aryl (e.g., phenyl) substituted with 1 R⁵ (e.g., —NH₂). In some embodiments, the compound of Formula (III-b) is Compound 102.

In some embodiments, the compound is a compound of Formula (III-b). In some embodiments of Formula (III-b), each of R^{2a}, R^{2b}, R^{2c}, and R^{2d} is independently hydrogen, m is 1, n is 2, q is 3, p is 0, R^{C} is hydrogen, and Z² is heterocyclyl (e.g., a nitrogen-containing heterocyclyl, e.g., a nitrogen-containing spiro heterocyclyl, e.g., 2-oxa-7-azaspiro[3.5]nonanyl). In some embodiments, the compound of Formula (III-a) is Compound 121.

In some embodiments, the compound is a compound of Formula (III-d). In some embodiments of Formula (III-d), each of R^{2a}, R^{2b}, R^{2c}, and R^{2d} is independently hydrogen, m is 1, n is 2, q is 1, 2, 3, or 4, p is 0, and X is S(O)₂. In some embodiments of Formula (III-d), each of R^{2a} and R^{2b} is independently hydrogen, m is 1, n is 2, q is 1, 2, 3, or 4, p is 0, and X is S(O)₂. In some embodiments, the compound of Formula (III-d) is Compound 101, Compound 117, Compound 118, or Compound 119.

In some embodiments, the compound is a compound of Formula (III-d). In some embodiments of Formula (III-i), each of R^{2a}, R^{2b}, R^{2c}, and R^{2d} is independently hydrogen, m is 1, n is 2, q is 3, p is 0, X is S(O)₂ and R¹² is deuterium, halogen (e.g., fluoro, chloro, or bromo), alkyl (e.g., methyl or ethyl), heteroalkyl (e.g., fluoromethyl, difluoromethyl, or trifluoromethyl). In some embodiments, the compound of Formula (III-d) is Compound 122, Compound 128, Compound 129, Compound 130, Compound 131, Compound 132, Compound 133, Compound 134, or Compound 135.

In some embodiments, the compound is a compound of Formula (III-d). In some embodiments of Formula (III-d), each of R^{2a}, R^{2b}, R^{2c}, and R^{2d} is independently hydrogen, m is 1, n is 2, q is 4, p is 0, X is S(O)₂, R¹² is deuterium, halogen (e.g., fluoro, chloro, or bromo), alkyl (e.g., methyl or ethyl), heteroalkyl (e.g., fluoromethyl, difluoromethyl, or trifluoromethyl). In some embodiments, the compound of Formula (III-d) is Compound 136, Compound 137, Compound 138, or Compound 143.

In some embodiments, the compound is a compound of Formula (III-d). In some embodiments of Formula (III-d), each of R^{2a}, R^{2b}, R^{2c}, and R^{2d} is independently hydrogen, m is 1, n is 2, q is 2, p is 0, X is S(O)₂, R¹² is deuterium, halogen (e.g., fluoro, chloro, or bromo), alkyl (e.g., methyl or ethyl), heteroalkyl (e.g., fluoromethyl, difluoromethyl, or trifluoromethyl). In some embodiments, the compound of Formula (III-d) is Compound 140, Compound 141, Compound 142, or Compound 139.

In some embodiments, the compound is a compound of Formula (I-b), (I-d), or (I-e). In some embodiments, the compound is a compound of Formula (I-b), (I-d), or (II). In some embodiments, the compound is a compound of Formula (I-b), (I-d), or (I-f). In some embodiments, the compound is a compound of Formula (I-b), (I-d), or (III).

Exemplary compounds of Formula (I) may be prepared as described in WO 2019/169333 or any other method known to those skilled in the art.

In some embodiments, the compound of Formula (I) is not a compound disclosed in WO 2012/112982, WO 2012/167223, WO 2014/153126, WO 2016/019391, WO 2017/075630, US 2012-0213708, US 2016-0030359 or US 2016-0030360.

In some embodiments, the compound of Formula (I) comprises a compound shown in Table 3, or a pharmaceutically acceptable salt thereof. In some embodiments, a device described herein comprises a compound shown in Table 3, or a pharmaceutically acceptable salt thereof.

TABLE 3
Exemplary compounds of Formula (I)

| Compound No. | Structure |
|---|---|
| 100 | 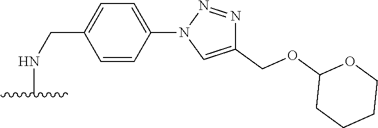 [SMILES: *NCc1ccc(-n2cc(COC3CCCCO3)nn2)cc1] |
| 101 | 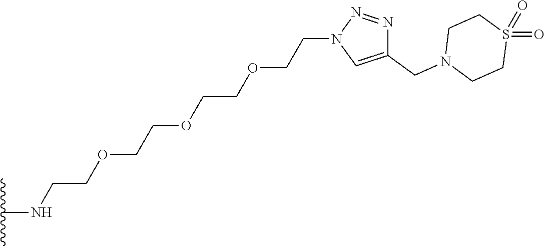 [SMILES: *NCCOCCOCCOCCn1cc(CN2CCS(=O)(=O)CC2)nn1] |
| 102 | 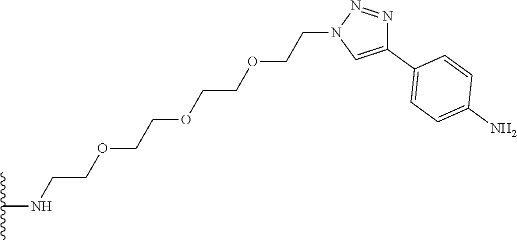 [SMILES: *NCCOCCOCCOCCn1cc(-c2ccc(N)cc2)nn1] |
| 103 | 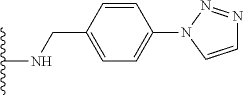 [SMILES: *NCc1ccc(-n2ccnn2)cc1] |
| 104 | 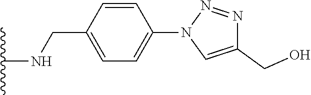 [SMILES: *NCc1ccc(-n2cc(CO)nn2)cc1] |
| 105 | 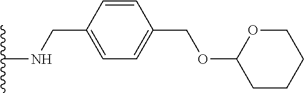 [SMILES: *NCc1ccc(COC2CCCCO2)cc1] |
| 106 | 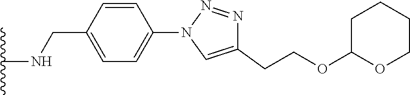 [SMILES: *NCc1ccc(-n2cc(CCOC3CCCCO3)nn2)cc1] |
| 107 | 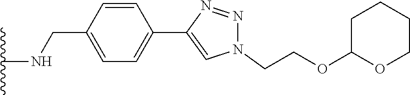 [SMILES: *NCc1ccc(-c2cn(CCOC3CCCCO3)nn2)cc1] |
| 108 | 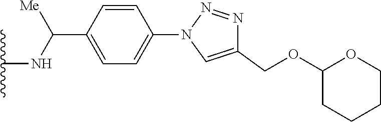 [SMILES: *NC(C)c1ccc(-n2cc(COC3CCCCO3)nn2)cc1] |
| 109 | 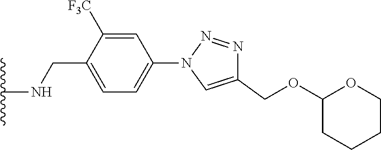 [SMILES: *NCc1ccc(-n2cc(COC3CCCCO3)nn2)cc1C(F)(F)F] |
| 110 | 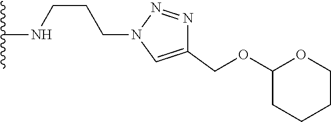 [SMILES: *NCCCn1cc(COC2CCCCO2)nn1] |
| 111 | 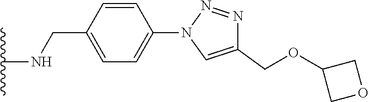 [SMILES: *NCc1ccc(-n2cc(COC3COC3)nn2)cc1] |
| 112 | 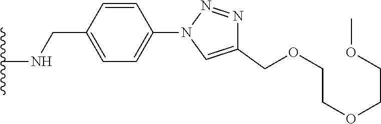 [SMILES: *NCc1ccc(-n2cc(COCCOCCOC)nn2)cc1] |
| 113 | 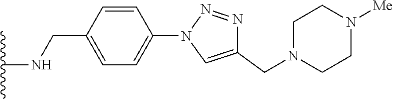 [SMILES: *NCc1ccc(-n2cc(CN3CCN(C)CC3)nn2)cc1] |
| 114 | 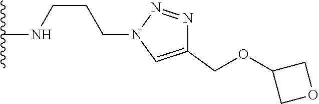 [SMILES: *NCCCn1cc(COC2COC2)nn1] |
| 115 | 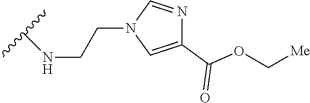 [SMILES: *NCCn1cnc(C(=O)OCC)c1] |
| 116 | 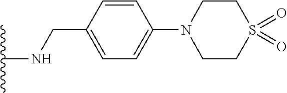 [SMILES: *NCc1ccc(N2CCS(=O)(=O)CC2)cc1] |
| 117 | 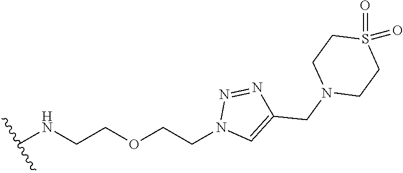 [SMILES: *NCCOCCn1cc(CN2CCS(=O)(=O)CC2)nn1] |
| 118 | 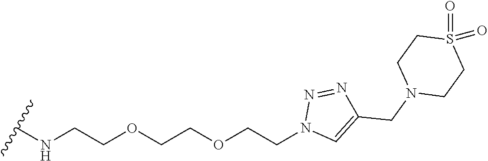 [SMILES: *NCCOCCOCCn1cc(CN2CCS(=O)(=O)CC2)nn1] |
| 119 | 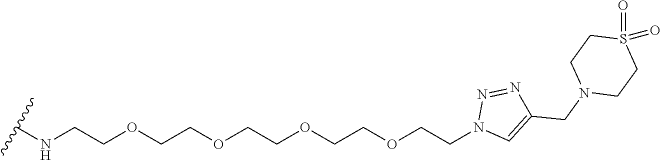 [SMILES: *NCCOCCOCCOCCOCCn1cc(CN2CCS(=O)(=O)CC2)nn1] |
| 120 | 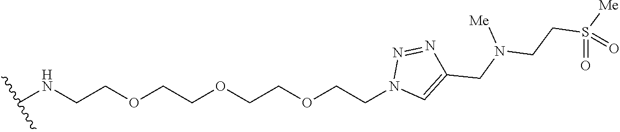 [SMILES: *NCCOCCOCCOCCn1cc(CN(C)CCS(C)(=O)=O)nn1] |
| 121 | 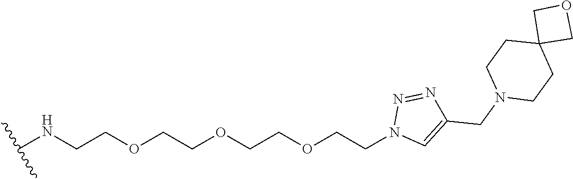 [SMILES: *NCCOCCOCCOCCn1cc(CN2CCC3(CC2)COC3)nn1] |
| 122 | 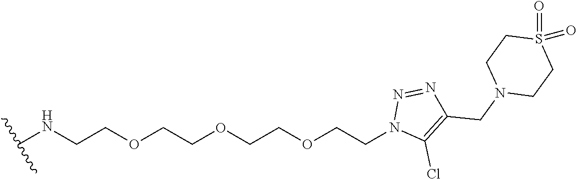 [SMILES: *NCCOCCOCCOCCn1nnc(CN2CCS(=O)(=O)CC2)c1Cl] |
| 123 | 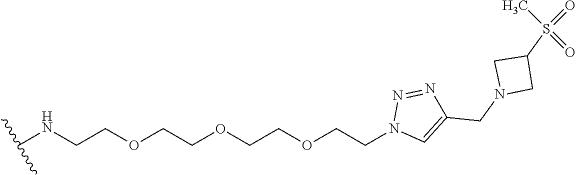 [SMILES: *NCCOCCOCCOCCn1cc(CN2CC(S(C)(=O)=O)C2)nn1] |
| 125 | 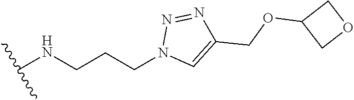 [SMILES: *NCCCn1cc(COC2COC2)nn1] |
| 126 | 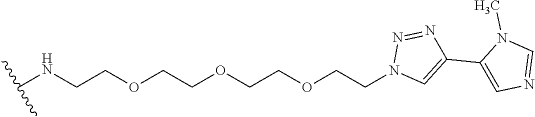 [SMILES: *NCCOCCOCCOCCn1cc(-c2cncn2C)nn1] |
| 128 | 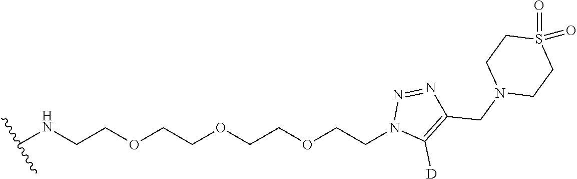 [SMILES: *NCCOCCOCCOCCn1nnc(CN2CCS(=O)(=O)CC2)c1[2H]] |
| 129 | 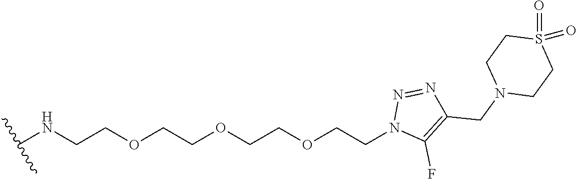 [SMILES: *NCCOCCOCCOCCn1nnc(CN2CCS(=O)(=O)CC2)c1F] |
| 130 | 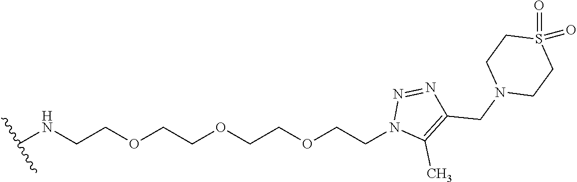 [SMILES: *NCCOCCOCCOCCn1nnc(CN2CCS(=O)(=O)CC2)c1C] |
| 131 | 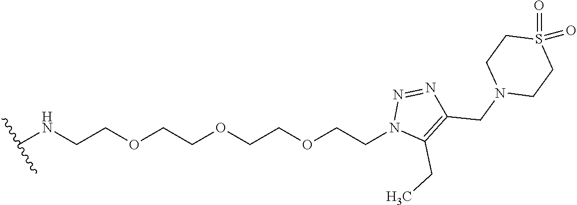 [SMILES: *NCCOCCOCCOCCn1nnc(CN2CCS(=O)(=O)CC2)c1CC] |
| 132 | 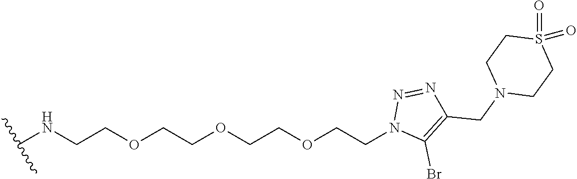 [SMILES: *NCCOCCOCCOCCn1nnc(CN2CCS(=O)(=O)CC2)c1Br] |
| 133 | 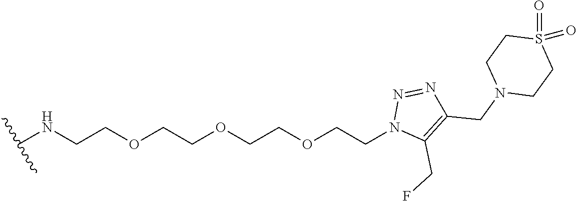 [SMILES: *NCCOCCOCCOCCn1nnc(CN2CCS(=O)(=O)CC2)c1CF] |
| 134 | 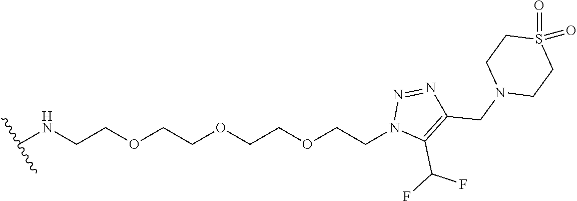 [SMILES: *NCCOCCOCCOCCn1nnc(CN2CCS(=O)(=O)CC2)c1C(F)F] |
| 135 | 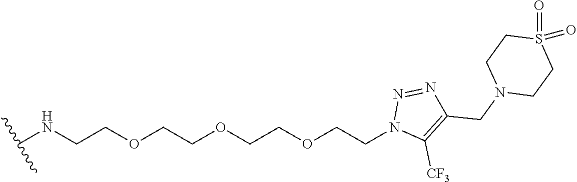 [SMILES: *NCCOCCOCCOCCn1nnc(CN2CCS(=O)(=O)CC2)c1C(F)(F)F] |
| 136 | 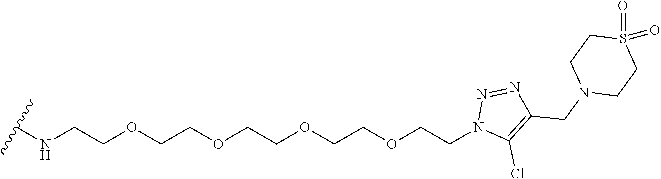 [SMILES: *NCCOCCOCCOCCOCCn1nnc(CN2CCS(=O)(=O)CC2)c1Cl] |
| 137 | 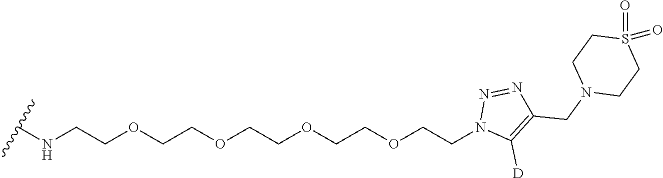 [SMILES: *NCCOCCOCCOCCOCCn1nnc(CN2CCS(=O)(=O)CC2)c1[2H]] |
| 138 | 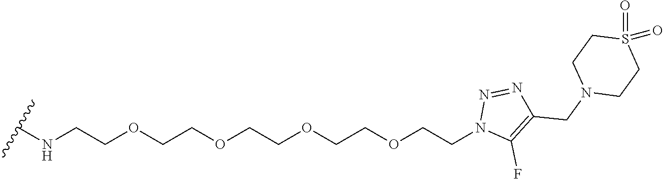 [SMILES: *NCCOCCOCCOCCOCCn1nnc(CN2CCS(=O)(=O)CC2)c1F] |
| 139 | 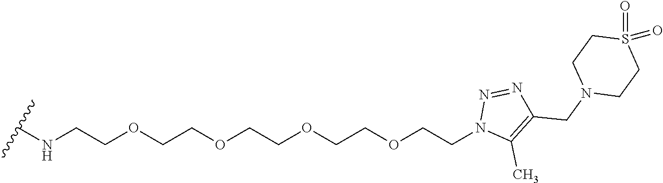 [SMILES: *NCCOCCOCCOCCOCCn1nnc(CN2CCS(=O)(=O)CC2)c1C] |
| 140 | 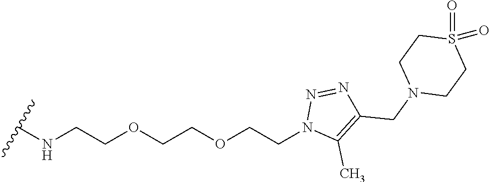 [SMILES: *NCCOCCOCCn1nnc(CN2CCS(=O)(=O)CC2)c1C] |
| 141 | 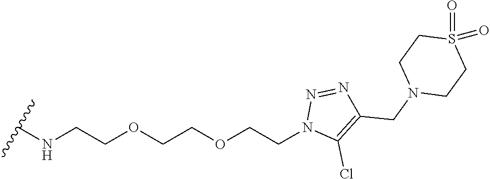 [SMILES: *NCCOCCOCCn1nnc(CN2CCS(=O)(=O)CC2)c1Cl] |
| 142 | 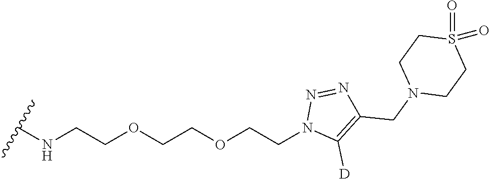 [SMILES: *NCCOCCOCCn1nnc(CN2CCS(=O)(=O)CC2)c1[2H]] |
| 143 | 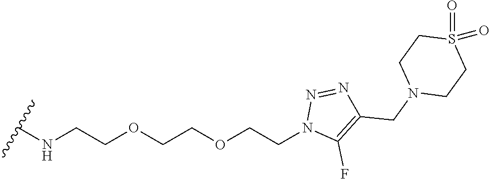 [SMILES: *NCCOCCOCCn1nnc(CN2CCS(=O)(=O)CC2)c1F] |
| 144 | 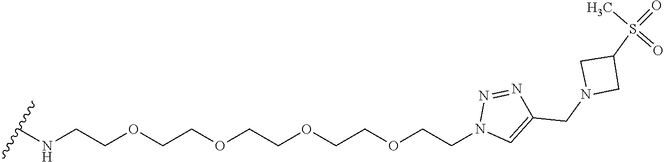 [SMILES: *NCCOCCOCCOCCOCCn1cc(CN2CC(S(C)(=O)=O)C2)nn1] |
| 145 | 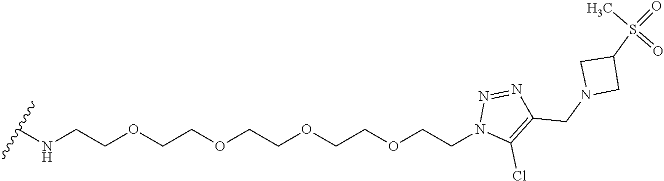 [SMILES: *NCCOCCOCCOCCOCCn1nnc(CN2CC(S(C)(=O)=O)C2)c1Cl] |
| 146 | 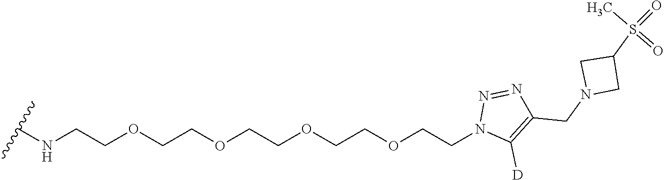 [SMILES: *NCCOCCOCCOCCOCCn1nnc(CN2CC(S(C)(=O)=O)C2)c1[2H]] |
| 147 | 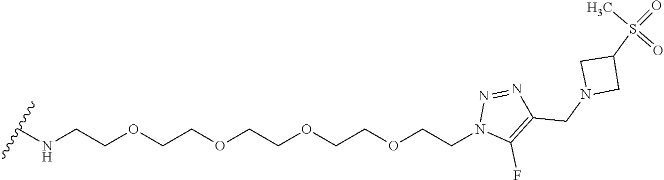 [SMILES: *NCCOCCOCCOCCOCCn1nnc(CN2CC(S(C)(=O)=O)C2)c1F] |
| 148 | 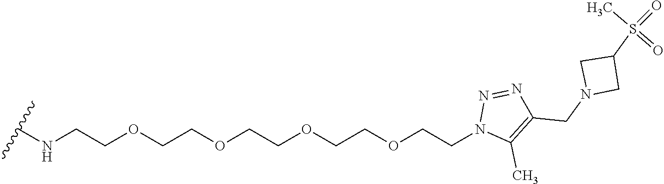 [SMILES: *NCCOCCOCCOCCOCCn1nnc(CN2CC(S(C)(=O)=O)C2)c1C] |
| 149 | 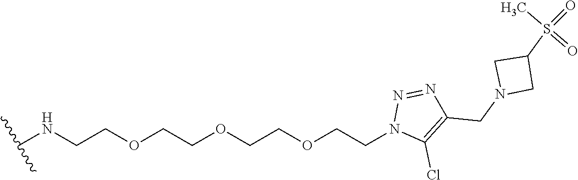 [SMILES: *NCCOCCOCCOCCn1nnc(CN2CC(S(C)(=O)=O)C2)c1Cl] |
| 150 | 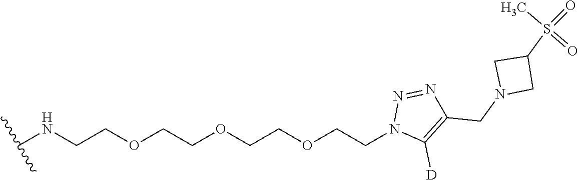 [SMILES: *NCCOCCOCCOCCn1nnc(CN2CC(S(C)(=O)=O)C2)c1[2H]] |
| 151 | 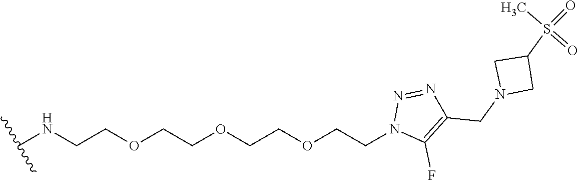 [SMILES: *NCCOCCOCCOCCn1nnc(CN2CC(S(C)(=O)=O)C2)c1F] |
| 152 | 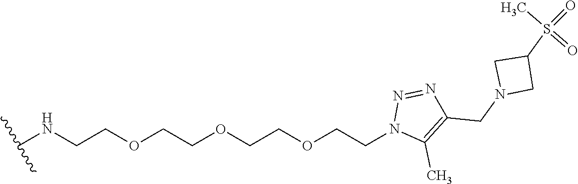 [SMILES: *NCCOCCOCCOCCn1nnc(CN2CC(S(C)(=O)=O)C2)c1C] |
| 153 | 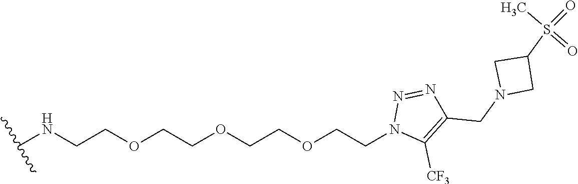 [SMILES: *NCCOCCOCCOCCn1nnc(CN2CC(S(C)(=O)=O)C2)c1C(F)(F)F] |
| 154 | 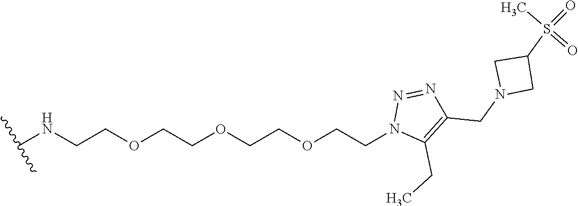 [SMILES: *NCCOCCOCCOCCn1nnc(CN2CC(S(C)(=O)=O)C2)c1CC] |

In some embodiments, the compound is a compound of Formula (I) (e.g., Formulas (I-a), (I-b), (I-b-i), (I-b-ii), (I-c), (I-d), (I-e), (I-f), (II), (II-a), (III), (III-a), (III-b), (III-c), (III-d), (III-e), (III-f), (III-g), or (III-i)), or a pharmaceutically acceptable salt thereof, and is selected from:

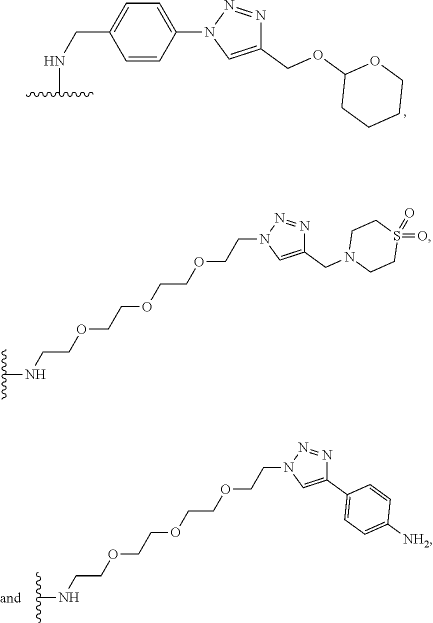 [SMILES: *NCc1ccc(-n2cc(COC3CCCCO3)nn2)cc1 ; *NCCOCCOCCOCCn1cc(CN2CCS(=O)(=O)CC2)nn1 ; *NCCOCCOCCOCCn1cc(-c2ccc(N)cc2)nn1 ; "and"]

or a pharmaceutically acceptable salt thereof.

In some embodiments, the device described herein comprises the compound of

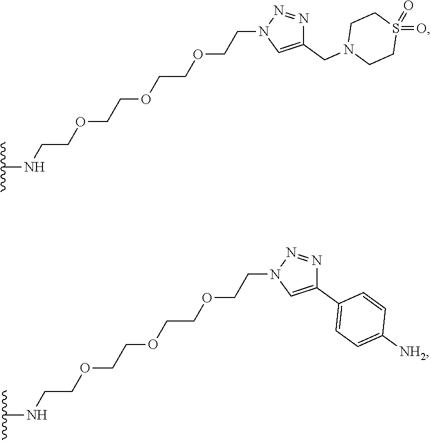 [SMILES: *NCCOCCOCCOCCn1cc(CN2CCS(=O)(=O)CC2)nn1 ; *NCCOCCOCCOCCn1cc(-c2ccc(N)cc2)nn1]

or a pharmaceutically acceptable salt thereof.

In an embodiment, a device described herein comprises a compound of Formula (I) (e.g., a compound shown in Table 3) covalently bound to an alginate polymer. The alginate polymer can be chemically modified with a compound of Formula (I) using any suitable method known in the art, e.g., as described in WO 2019/195055.

### Methods of Treatment

Described herein are methods for preventing or treating a disease, disorder, or condition in a subject by administering to the subject a plurality of genetically modified cells described herein that produce a therapeutic agent that treats the disease, disorder or condition. The cells may be administered by implanting into the subject a device containing the cells as described herein, or a preparation of such devices. In an embodiment, the device or preparation is implanted (e.g., via laparoscopy) into the intraperitoneal space, e.g., the greater sac of the peritoneal cavity. In an embodiment, the genetically modified cells are engineered RPE cells, and the method comprises administering (e.g., implanting) an effective amount of a composition of two-compartment alginate hydrogel capsules which comprise the engineered RPE cells and a cell-binding polymer described herein in the inner compartment and comprise a Compound of Formula (I), e.g., Compound 101, on the outer capsule surface. In some embodiments, the method of treatment directly or indirectly reduces or alleviates at least one symptom of the disease, disorder, or condition and/or the method prevents or slows the onset of the disease, disorder, or condition. In some embodiments, the subject is a human.

In some embodiments, the disease, disorder, or condition affects a system of the body, e.g., the nervous system (e.g., peripheral nervous system (PNS) or central nervous system (CNS)), vascular system, skeletal system, respiratory system, endocrine system, lymph system, reproductive system, or gastrointestinal tract. In some embodiments, the disease, disorder, or condition affects a part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis.

In some embodiments, the disease, disorder or condition is a neurodegenerative disease, diabetes, a heart disease, an autoimmune disease, a cancer, a liver disease, a lysosomal storage disease, a blood clotting disorder or a coagulation disorder, an orthopedic condition, an amino acid metabolism disorder.

In some embodiments, the disease, disorder or condition is a neurodegenerative disease. Exemplary neurodegenerative diseases include Alzheimer's disease, Huntington's disease, Parkinson's disease (PD) amyotrophic lateral sclerosis (ALS), multiple sclerosis (MS) and cerebral palsy (CP), dentatorubro-pallidoluysian atrophy (DRPLA), neuronal intranuclear hyaline inclusion disease (NIHID), dementia with Lewy bodies, Down's syndrome, Hallervorden-Spatz disease, prion diseases, argyrophilic grain dementia, cortocobasal degeneration, dementia pugilistica, diffuse neurofibrillary tangles, Gerstmann-Straussler-Scheinker disease, Jakob-Creutzfeldt disease, Niemann-Pick disease type 3, progressive supranuclear palsy, subacute sclerosing panencephalitis, spinocerebellar ataxias, Pick's disease, and dentatorubral-pallidoluysian atrophy.

In some embodiments, the disease, disorder, or condition is an autoimmune disease, e.g., scleroderma, multiple sclerosis, lupus, or allergies.

In some embodiments, the disease is a liver disease, e.g., hepatitis B, hepatitis C, cirrhosis, NASH.

In some embodiments, the disease, disorder, or condition is cancer. Exemplary cancers include leukemia, lymphoma, melanoma, lung cancer, brain cancer (e.g., glioblastoma), sarcoma, pancreatic cancer, renal cancer, liver cancer, testicular cancer, prostate cancer, or uterine cancer.

In some embodiments, the disease, disorder, or condition is an orthopedic condition. Exemplary orthopedic conditions include osteoporosis, osteonecrosis, Paget's disease, or a fracture.

In some embodiments, the disease, disorder or condition is a lysosomal storage disease. Exemplary lysosomal storage diseases include Gaucher disease (e.g., Type I, Type II, Type III), Tay-Sachs disease, Fabry disease, Farber disease, Mucopolysaccharidosis type I (MPS I) (also known as Hurler syndrome), Hunter syndrome, lysosomal acid lipase deficiency, Niemann-Pick disease, Salla disease, Sanfilippo syndrome (also known as mucopolysaccharidosis type IIIA (MPS3A)), multiple sulfatase deficiency, Maroteaux-Lamy syndrome, metachromatic leukodystrophy, Krabbe disease, Scheie syndrome, Hurler-Scheie syndrome, Sly syndrome, hyaluronidase deficiency, Pompe disease, Danon disease, gangliosidosis, or Morquio syndrome.

In some embodiments, the disease, disorder, or condition is a blood clotting disorder or a coagulation disorder. Exemplary blood clotting disorders or coagulation disorders include hemophilia (e.g., hemophilia A or hemophilia B), Von Willebrand disease, thrombocytopenia, uremia, Bernard-Soulier syndrome, Factor XII deficiency, vitamin K deficiency, or congenital afibrinogenimia.

In some embodiments, the disease, disorder, or condition is an amino acid metabolism disorder, e.g., phenylketonuria, tyrosinemia (e.g., Type 1 or Type 2), alkaptonuria, homocystinuria, hyperhomocysteinemia, maple syrup urine disease.

In some embodiments, the disease, disorder, or condition is a fatty acid metabolism disorder, e.g., hyperlipidemia, hypercholesterolemia, galactosemia.

In some embodiments, the disease, disorder, or condition is a purine or pyrimidine metabolism disorder, e.g., Lesch-Nyhan syndrome.

In some embodiments, the disease, disorder, or condition is diabetes (e.g., Type I or Type II diabetes). In some embodiments, the disease, disorder or condition is not Type I diabetes. In some embodiments, the disease, disorder or condition is not Type II diabetes.

### EXEMPLARY ENUMERATED EMBODIMENTS

1. A genetically modified cell derived from a human cell and comprising at least one exogenous transcription unit in at least one genomic insertion site (GIS) selected from the group consisting of:
   (a) a Chr 1 GIS located between two contiguous or non-contiguous nucleotide positions x and y within SEQ ID NO:1 or a human genomic nucleotide sequence that is at least 90%, 95%, 98%, or 99% identical to SEQ ID NO:1;
   (b) a first Chr 2 GIS located between two contiguous or non-contiguous nucleotide positions x and y within SEQ ID NO:2 or a human genomic nucleotide sequence that is at least 90%, 95%, 98%, or 99% identical to SEQ ID NO:2;
   (c) a second Chr 2 GIS located between two contiguous or non-contiguous nucleotide positions x and y within SEQ ID NO:3 or a human genomic nucleotide sequence that is at least 90%, 95%, 98%, or 99% identical to SEQ ID NO:3;
   (d) a first Chr 3 GIS located between two contiguous or non-contiguous nucleotide positions x and y within SEQ ID NO:4 or a human genomic nucleotide sequence that is at least 90%, 95%, 98%, or 99% identical to SEQ ID NO:4;
   (e) a second Chr 3 GIS located between two contiguous or non-contiguous nucleotide positions x and y within SEQ ID NO:5 or a human genomic nucleotide sequence that is at least 90%, 95%, 98%, or 99% identical to SEQ ID NO:5;
   (f) a third Chr 3 GIS located between two contiguous or non-contiguous nucleotide positions x and y within SEQ ID NO:6 or a human genomic nucleotide sequence that is at least 90%, 95%, 98%, or 99% identical to SEQ ID NO:6;
   (g) a first Chr 4 GIS located between two contiguous or non-contiguous nucleotide positions x and y within SEQ ID NO:7 or a human genomic nucleotide sequence that is at least 90%, 95%, 98%, or 99% identical to SEQ ID NO:7;
   (h) a second Chr 4 GIS located between two contiguous or non-contiguous nucleotide positions x and y within SEQ ID NO:8 or a human genomic nucleotide sequence that is at least 90%, 95%, 98%, or 99% identical to SEQ ID NO:8;
   (i) a first Chr 5 GIS located between two contiguous or non-contiguous nucleotide positions x and y within SEQ ID NO:9 or a human genomic nucleotide sequence that is at least 90%, 95%, 98%, or 99% identical to SEQ ID NO:9;
   (j) a second Chr 5 GIS located between two contiguous or non-contiguous nucleotide positions x and y within SEQ ID NO:10 or a human genomic nucleotide sequence that is at least 90%, 95%, 98%, or 99% identical to SEQ ID NO:10;
   (k) a first Chr 6 GIS located between two contiguous or non-contiguous nucleotide positions x and y within SEQ ID NO:11 or a human genomic nucleotide sequence that is at least 90%, 95%, 98%, or 99% identical to SEQ ID NO: 11;
   (l) a second Chr 6 GIS located between two contiguous or non-contiguous nucleotide positions x and y within SEQ ID NO:12 or a human genomic nucleotide sequence that is at least 90%, 95%, 98%, or 99% identical to SEQ ID NO:12;
   (m) a third Chr 6 GIS located between two contiguous or non-contiguous nucleotide positions x and y within SEQ ID NO:13 or of a human genomic nucleotide sequence that is at least 90%, 95%, 98%, or 99% identical to SEQ ID NO: 13;
   (n) a fourth Chr 6 GIS located between two contiguous or non-contiguous nucleotide positions x and y within SEQ ID NO:14 or a human genomic nucleotide sequence that is at least 90%, 95%, 98%, or 99% identical to SEQ ID NO:14;
   (o) a fifth Chr 6 GIS located between two contiguous or non-contiguous nucleotide positions x and y within SEQ ID NO:15 or a human genomic nucleotide sequence that is at least 90%, 95%, 98%, or 99% identical to SEQ ID NO: 15;
   (p) a first Chr 7 GIS located between two contiguous or non-contiguous nucleotide positions x and y within SEQ ID NO:16 or a human genomic nucleotide sequence that is at least 90%, 95%, 98%, or 99% identical to SEQ ID NO: 16;
   (q) a second Chr 7 GIS located between two contiguous or non-contiguous nucleotide positions x and y within SEQ ID NO:17 or a human genomic nucleotide sequence that is at least 90%, 95%, 98%, or 99% identical to SEQ ID NO:17;
   (r) a Chr 8 GIS located between two contiguous or non-contiguous nucleotide positions x and y within SEQ ID NO:18 or a human genomic nucleotide sequence that is at least 90%, 95%, 98%, or 99% identical to SEQ ID NO:18;
   (s) a first Chr 9 GIS located between two contiguous or non-contiguous nucleotide positions x and y within SEQ ID NO:19 or a human genomic nucleotide sequence that is at least 90%, 95%, 98%, or 99% identical to SEQ ID NO: 19;
   (t) a second Chr 9 GIS located between two contiguous or non-contiguous nucleotide positions x and y within SEQ ID NO:20 or a human genomic nucleotide sequence that is at least 90%, 95%, 98%, or 99% identical to SEQ ID NO:20;
   (u) a Chr 10 GIS located between two contiguous or non-contiguous nucleotide positions x and y within SEQ ID NO:21 or a human genomic nucleotide sequence that is at least 90%, 95%, 98%, or 99% identical to SEQ ID NO:21;
   (v) a Chr 13 GIS located between two contiguous or non-contiguous nucleotide positions x and y within SEQ ID NO:22 or a human genomic nucleotide sequence that is at least 90%, 95%, 98%, or 99% identical to SEQ ID NO:22; and
   (w) a Chr 14 GIS located between two contiguous or non-contiguous nucleotide positions x and y within SEQ ID NO:23 or a human genomic nucleotide sequence that is at least 90%, 95%, 98%, or 99% identical to SEQ ID NO:23;
      optionally wherein for each GIS defined in (a) to (w), the x and y positions are independently selected from the group consisting of:
      x and y are 100 and 1700;
      x and y are 200 and 1600;
      x and y are 400 and 1400;
      x and y are 800 and 1000;
      x and y are 850 and 950;
      x and y are 875 and 925;
      x and y are 890 and 910; and
      x and y are 898 and 903.
2. The genetically modified cell of embodiment 1, wherein the genomic insertion site (GIS) comprises (a).
3. The genetically modified cell of any one of embodiments 1-2, wherein the genomic insertion site (GIS) comprises (b).
4. The genetically modified cell of any one of embodiments 1-3, wherein the genomic insertion site (GIS) comprises (c).
5. The genetically modified cell of any one of embodiments 1-4, wherein the genomic insertion site (GIS) comprises (d).
6. The genetically modified cell of any one of embodiments 1-5, wherein the genomic insertion site (GIS) comprises (e).
7. The genetically modified cell of any one of embodiments 1-6, wherein the genomic insertion site (GIS) comprises (f).
8. The genetically modified cell of any one of embodiments 1-7, wherein the genomic insertion site (GIS) comprises (f).
9. The genetically modified cell of any one of embodiments 1-8, wherein the genomic insertion site (GIS) comprises (g).
10. The genetically modified cell of any one of embodiments 1-9, wherein the genomic insertion site (GIS) comprises (h).
11. The genetically modified cell of any one of embodiments 1-10, wherein the genomic insertion site (GIS) comprises (i).
12. The genetically modified cell of any one of embodiments 1-11, wherein the genomic insertion site (GIS) comprises (j).
13. The genetically modified cell of any one of embodiments 1-12, wherein the genomic insertion site (GIS) comprises (k).
14. The genetically modified cell of any one of embodiments 1-13, wherein the genomic insertion site (GIS) comprises (l).
15. The genetically modified cell of any one of embodiments 1-14, wherein the genomic insertion site (GIS) comprises (m).
16. The genetically modified cell of any one of embodiments 1-15, wherein the genomic insertion site (GIS) comprises (n).
17. The genetically modified cell of any one of embodiments 1-16, wherein the genomic insertion site (GIS) comprises (o).
18. The genetically modified cell of any one of embodiments 1-17, wherein the genomic insertion site (GIS) comprises (p).
19. The genetically modified cell of any one of embodiments 1-18, wherein the genomic insertion site (GIS) comprises (q).
20. The genetically modified cell of any one of embodiments 1-19, wherein the genomic insertion site (GIS) comprises (r).
21. The genetically modified cell of any one of embodiments 1-20, wherein the genomic insertion site (GIS) comprises (s).
22. The genetically modified cell of any one of embodiments 1-21, wherein the genomic insertion site (GIS) comprises (t).
23. The genetically modified cell of any one of embodiments 1-22, wherein the genomic insertion site (GIS) comprises (u).
24. The genetically modified cell of any one of embodiments 1-23, wherein the genomic insertion site (GIS) comprises (v).
25. The genetically modified cell of any one of embodiments 1-24, wherein the genomic insertion site (GIS) comprises (v).
26. The genetically modified cell of any one of embodiments 1-25, wherein the exogenous transcription unit is inserted into any two, three, four or five of the genomic insertion sites.
27. The genetically modified cell of any one of embodiments 1-26, wherein the exogenous transcription unit is inserted into any six, seven, eight or nine of the genomic insertion sites.
28. The genetically modified cell of any one of embodiments 1-27, wherein the exogenous transcription unit is inserted into any 10, 11, 12, or 13 of the genomic insertion sites.
29. The genetically modified cell of any one of embodiments 1-28, wherein the exogenous transcription unit is inserted into any 14, 15, 16 or 17 of the genomic insertion sites.
30. The genetically modified cell of any one of embodiments 1-29, wherein the exogenous transcription unit is inserted into any 18, 19, 20, 21 or 22 of the genomic insertion sites.
31. The genetically modified cell of any one of embodiments 1-30, wherein the exogenous transcription unit is inserted into each of the 23 genomic insertion sites.
32. The genetically modified cell of any one of embodiments 1-31, wherein the exogenous transcription unit is inserted into each of five genomic insertion sites, which are the Chr 1 GIS, the first Chr 3 GIS, the fourth Chr 6 GIS, the first Chr 7 GIS and the second Chr 9 GIS.
33. The genetically modified cell of any one of embodiments 1-32, wherein the exogenous transcription unit is inserted into each of 18 genomic insertion sites, which are the first and second Chr 2 GIS, the second and third Chr 3 GIS, the first and second Chr 4 GIS, the first and second Chr 5 GIS, the first, second, third and fifth Chr 6 GIS, the second Chr 7 GIS, the Chr 8 GIS, the first Chr 9 GIS, the Chr 10 GIS, the Chr 13 GIS and the Chr 14 GIS.
34. The genetically modified cell of any one of embodiments 1 to 9, wherein the cell further comprises the exogenous transcription unit in at least one OCR that does not comprise any of SEQ ID Nos:1-23.
35. The genetically modified cell of any one of embodiments 1 to 10, wherein positions x and y are 890 and 910 for one or more of the genomic insertion sites of embodiment 1.
36. The genetically modified cell of any one of embodiments 1 to 11, wherein positions x and y are 898 and 903 for one or more of the genomic insertion sites of embodiment 1.
37. The genetically modified cell of any one of embodiments, 1 to 12, wherein x and y are 898 and 903 for at least two, three, four, five or more of the genomic insertion sites of embodiment 1.
38. The genetically modified cell of embodiment 8, wherein x and y are 898 and 903 for each of the five genomic insertion sites.
39. The genetically modified cell of embodiment 9, wherein x and y are 898 and 903 for each of the 18 genomic insertion sites.
40. The genetically modified cell of any one of embodiments 1 to 39, which is derived from a human immortalized cell.
41. The genetically modified cell of any one of embodiments 1 to 40, which is derived from a human retinal epithelial cell line.
42. The genetically modified epithelial cell of embodiment 41, wherein the human retinal epithelial cell line is the ARPE-19 cell line.
43. The genetically modified cell of any one of embodiments 1 to 41, wherein the exogenous transcription unit comprises a promoter sequence operably linked to a coding sequence for a polypeptide, optionally wherein the polypeptide is selected from the group consisting of: an FVII protein, an FVIII protein, a FIX protein, a GLA protein and an IDUA protein.
44. The genetically modified cell of any one of embodiments 1 to 43, wherein the exogenous transcription unit comprises a promoter sequence operably linked to a coding sequence for a polypeptide, wherein the polypeptide is an FVII protein.
45. The genetically modified cell of any one of embodiments 1 to 43, wherein the exogenous transcription unit comprises a promoter sequence operably linked to a coding sequence for a polypeptide, wherein the polypeptide is an FVIII protein.
46. The genetically modified cell of any one of embodiments 1 to 43, wherein the exogenous transcription unit comprises a promoter sequence operably linked to a coding sequence for a polypeptide, wherein the polypeptide is a FIX protein.
47. The genetically modified cell of any one of embodiments 1 to 43, wherein the exogenous transcription unit comprises a promoter sequence operably linked to a coding sequence for a polypeptide, wherein the polypeptide is an a GLA protein.
48. The genetically modified cell of any one of embodiments 1 to 43, wherein the exogenous transcription unit comprises a promoter sequence operably linked to a coding sequence for a polypeptide, wherein the polypeptide is an IDUA protein.
49. The genetically modified cell of any one of embodiments 43-48, wherein the promoter sequence consists essentially of or consists of SEQ ID NO:24 or SEQ ID NO:25.
50. The genetically modified cell of embodiment 49, wherein the promoter sequence consists essentially of or consists of SEQ ID NO:24.
51. The genetically modified cell of embodiment 49, wherein the promoter sequence consists essentially of or consists of SEQ ID NO:25.
52. The genetically modified cell of embodiment 49, wherein the coding sequence is operably linked to a polyA signal sequence which consists essentially of, or consists of, SEQ ID NO:26.
53. The genetically modified cell of embodiments 43, 47, and 49-52, wherein the polypeptide is a GLA protein and the exogenous transcription unit comprises SEQ ID NO: 29, SEQ ID NO:30, or SEQ ID NO:32.
54. The genetically modified cell of embodiments 43, 47, and 49-52, wherein the polypeptide is a GLA protein and the exogenous transcription unit comprises SEQ ID NO: 29.
55. The genetically modified cell of embodiments 43, 47, and 49-52, wherein the polypeptide is a GLA protein and the exogenous transcription unit comprises SEQ ID NO: 30.
56. The genetically modified cell of embodiments 43, 47, and 49-52, wherein the polypeptide is a GLA protein and the exogenous transcription unit comprises SEQ ID NO: 32.
57. The genetically modified cell of embodiment 53, wherein the exogenous transcription unit comprises, consists of, or consists essentially of SEQ ID NO:33.
58. A composition comprising a plurality of genetically modified cells, wherein each cell in the plurality is a genetically modified cell as defined by any one of embodiments 1 to 57.
59. The composition of embodiment 24, wherein the plurality of cells is obtained by culturing a monoclonal cell line.
60. The composition of embodiments 58-59, wherein the exogenous transcription unit encodes a GLA protein, and the plurality of cells produce at least any of 5 pg/cell/day or 10 pg/cell/day at least for at least 2 days, 4 days, 1 week or 2 weeks, as measured by an assay described herein.
61. The composition of any one of embodiments 58-60, which further comprises a storage medium, e.g., a culture media, a cryopreservation medium.
62. A device comprising the composition of any one of embodiments 58-61.
63. An implantable device which comprises at least one cell-containing compartment comprising a genetically modified cell or a plurality of the genetically modified cell and at least one means for mitigating the foreign body response (FBR) when the device is implanted into the subject, wherein the genetically modified cell or each cell in the plurality is a genetically modified cell as defined by any one of embodiments 1 to 62.
64. The device of embodiment 63, wherein the at least one cell-containing compartment comprises a polymer composition which encapsulates the plurality of engineered RPE cells, and optionally comprises at least one cell-binding substance (CBS).
65. The device of embodiment 63 or 64, wherein the cell-containing compartment comprises an alginate hydrogel and is surrounded by a barrier compartment, which comprises an alginate hydrogel and optionally comprises a compound of Formula (I), e.g., Compound 101, disposed on the outer surface of the barrier compartment.
66. The device of embodiment 63-65, wherein the cell-containing compartment comprises an alginate hydrogel and is surrounded by a barrier compartment, which comprises an alginate hydrogel, which comprises a compound of Formula (I) disposed on the outer surface of the barrier compartment.
67. The device of embodiment 63-66, wherein the cell-containing compartment comprises an alginate hydrogel and is surrounded by a barrier compartment, which comprises an alginate hydrogel, which comprises Compound 101 disposed on the outer surface of the barrier compartment.
68. The device of any one of embodiments 65-67, wherein the barrier compartment comprises an alginate chemically modified with Compound 101, and optionally wherein the alginate hydrogel in the barrier compartment is ionically cross-linked.
69. The device of any one of embodiments 65-68, wherein the barrier compartment comprises an alginate chemically modified with Compound 101, and wherein the alginate hydrogel in the barrier compartment is ionically cross-linked.
70. The device of any one of embodiments 63-69, wherein the polymer composition comprises an alginate covalently modified with a peptide, wherein the peptide consists essentially of or consists of GRGDSP (SEQ ID NO: 37), GGRGDSP (SEQ ID NO: 38) or GGGRGDSP (SEQ ID NO: 39).
71. The device of any one of embodiments 63-69, wherein the polymer composition comprises an alginate covalently modified with a peptide, wherein the peptide consists essentially of or consists of GRGDSP (SEQ ID NO: 37).
72. The device of any one of embodiments 63-69, wherein the polymer composition comprises an alginate covalently modified with a peptide, wherein the peptide consists essentially of or consists of GGRGDSP (SEQ ID NO: 38).
73. The device of any one of embodiments 63-69, wherein the polymer composition comprises an alginate covalently modified with a peptide, wherein the peptide consists essentially of or consists of GGGRGDSP (SEQ ID NO: 39).
74. The device of any one of embodiments 63-73, which is a hydrogel capsule of about 0.75 mm to about 2 mm in diameter, wherein the hydrogel capsule comprises an inner compartment surrounded by an outer compartment, wherein the genetically modified cells are contained in the inner compartment and the outer compartment is substantially free of the cells.
75. A preparation of devices, wherein each device in the preparation is a device as defined in any one of embodiments 62-74 and the preparation optionally comprises a pharmaceutically acceptable solution.
76. A method of treating a human subject for Fabry disease, comprising:
   providing a preparation of devices which contain a plurality of genetically modified cells expressing a GLA protein; and
   disposing the preparation in the body of the subject;
   wherein each cell in the plurality is the genetically modified cell of embodiment 53-57.
77. The method of embodiment 76, wherein the disposing step comprises placing the preparation into the intraperitoneal space.
78. The method of embodiment 76 or 77, wherein the disposing step comprises placing the preparation into the greater sac of the peritoneal cavity.

### EXAMPLES

In order that the disclosure described herein may be more fully understood, the following examples are set forth. The examples described in this application are offered to illustrate the genetically modified cells, compositions and implantable devices and methods provided herein, and are not to be construed in any way as limiting their scope.

### Example 1: Generation and Evaluation of Clones Expressing a GLA Protein

ARPE-19 cells were engineered to express a human GLA protein using the PiggyBac transposon system, which is capable of mediating transfer of a transcription unit between a plasmid vector and TTAA chromosomal sites through a “cut and paste” mechanism. ARPE-19 cells were split and seeded at 400,000 cells per 6-well culture plate and then co-transfected with varying amounts of two plasmids: (1) a transposon vector comprising a transcription unit encoding SEQ ID NO:31 inserted between inverted terminal repeat (ITR) elements recognized by a PiggyBac transposase and (2) a helper plasmid that expresses a piggyBac transposase enzyme and a fluorescent reporter protein (FRP). The transcription unit comprised SEQ ID NO:33.

Transfected pools were cultured in 6-well plates until they reached 75% confluency (2-3 days), the culture media was replaced with fresh media, cell supernatants were collected 24 hours later and assayed for GLA protein concentration using a GLA activity assay, substantially as described below.

A suspension of about 400,000 number of cells for each clone to be evaluated was added to wells of a 6-well plate in duplicate with 2 mL of complete growth medium (DMEM:F12 with 10% FBS and 1× Penicillin-Streptomycin-Neomycin antibiotics, Gibco). The plates were incubated for about 24 hours at 37° C. with 5% CO₂ and the amount of GLA protein secreted in vitro was quantitated by an enzymatic assay using a blue-fluorogenic substrate, 4-methylumbelliferyl-α-D-galactopyranoside. This assay measures the activity of GLA by measuring the formation of free 4-methylumbelliferyl as an increase in fluorescence at 460 nm emission when excited with 360 nm light. This activity in the cell culture media was compared to a standard curve generated with a recombinant GLA protein to determine the concentration of active GLA.

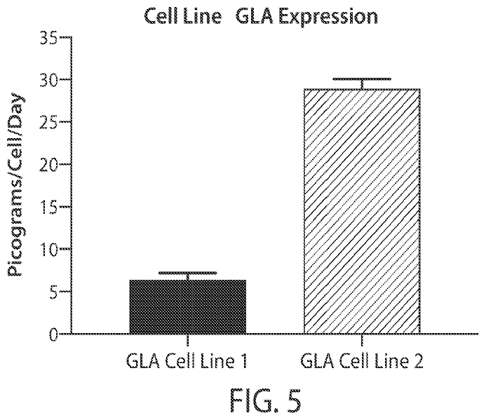

The pool with the highest GLA supernatant levels was selected for clone isolation and screening for GLA expression as described above, and the data for two clones with the highest GLA production are shown in FIG. 5. Each of clones 1 and 2 secreted GLA at levels exceeding a desired minimum of 5 pg/cell/day. Of 1500 clones that were evaluated, almost all failed to meet this desired minimum. The genomes of clones 1 and 2 were analyzed to determine the number and location of inserted transcription units, with the results shown in Table 1 above.

### Example 2: Culturing of Exemplary Genetically-Modified ARPE-19 Cells for Encapsulation

Genetically modified ARPE-19 cells comprising a stably integrated exogenous transcription unit as described herein may be cultured to produce a composition of cells suitable for encapsulation in two compartment hydrogel capsules. Cells are grown in complete growth medium (DMEM:F12 with 10% FBS) in 150 cm² cell culture flasks or CellSTACK® Culture Chambers (Corning Inc., Corning, NY). To passage cells, the medium in the culture flask is aspirated, and the cell layer is briefly rinsed with phosphate buffered saline (pH 7.4, 137 mM NaCl, 2.7 mM KCl, 8 mM Na₂HPO₄, and 2 mM KH₂PO₄, Gibco). 5-10 mL of 0.05% (w/v) trypsin/0.53 mM EDTA solution (“TrypsinEDTA”) is added to the flask, and the cells are observed under an inverted microscope until the cell layer is dispersed, usually between 3-5 minutes. To avoid clumping, cells are handled with care and hitting or shaking the flask during the dispersion period is minimized. If the cells do not detach, the flasks are placed at 37° C. to facilitate dispersal. Once the cells disperse, 10 mL complete growth medium is added and the cells are aspirated by gentle pipetting. The cell suspension is transferred to a centrifuge tube and spun down at approximately 125×g for 5-10 minutes to remove TrypsinEDTA. The supernatant is discarded, and the cells are resuspended in fresh growth medium. Appropriate aliquots of cell suspension are added to new culture vessels, which are incubated at 37° C. The medium is renewed weekly.

### Example 3: Preparation of Exemplary Modified Polymers

Chemically-modified Polymer. A polymeric material may be chemically modified with a compound of Formula (I) (or pharmaceutically acceptable salt thereof) prior to formation of a device described herein (e.g., a hydrogel capsule). For example, in the case of alginate, the alginate carboxylic acid is activated for coupling to one or more amine-functionalized compounds to achieve an alginate modified with an afibrotic compound, e.g., a compound of Formula (I). The alginate polymer is dissolved in water (30 mL/gram polymer) and treated with 2-chloro-4,6-dimethoxy-1,3,5-triazine (0.5 eq) and N-methylmorpholine (1 eq). To this mixture is added a 5 solution of the compound of interest (e.g., Compound 101 shown in Table 3) in acetonitrile (0.3M).

The amounts of the compound and coupling reagent added depends on the desired concentration of the compound bound to the alginate, e.g., conjugation density. A medium conjugation density of Compound 101 typically ranges from 2% to 5% N, while a high conjugation density of Compound 101 typically ranges from 5.1% to 8% N. To prepare a solution of low molecular weight alginate, chemically modified with a medium conjugation density of Compound 101 (CM-LMW-Alg-101-Medium polymer), the dissolved unmodified low molecular weight alginate (approximate MW<75 kDa, G:M ratio ≥1.5) is treated with 2-chloro-4,6-dimethoxy-1,3,5-triazine (5.1 mmol/g alginate) and N-methylmorpholine (10.2 mmol/g alginate) and Compound 101 (5.4 mmol/g alginate). To prepare a solution of low molecular weight alginate, chemically modified with a high conjugation density of Compound 101 (CM-LMW-Alg-101-High polymer), the dissolved unmodified low-molecular weight alginate (approximate MW<75 kDa, G:M ratio. 1.5) is treated with 2-chloro-4,6-dimethoxy-1,3,5-triazine (5.1 mmol/g alginate) and N-methylmorpholine (10.2 mmol/g alginate) and Compound 101 (10.5 mmol/g alginate).

The reaction is warmed to 55° C. for 16 h, then cooled to room temperature and gently concentrated via rotary evaporation, then the residue is dissolved in water. The mixture is filtered through a bed of cyano-modified silica gel (Silicycle) and the filter cake is washed with water. The resulting solution is then extensively dialyzed (10,000 MWCO membrane) and the alginate solution is concentrated via lyophilization to provide the desired chemically-modified alginate as a solid or is concentrated using any technique suitable to produce a chemically modified alginate solution with a viscosity of 25 cP to 35 cP.

The conjugation density of a chemically modified alginate is measured by combustion analysis for percent nitrogen. The sample is prepared by dialyzing a solution of the chemically modified alginate against water (10,000 MWCO membrane) for 24 hours, replacing the water twice followed by lyophilization to a constant weight.

CBP-Alginates. A polymeric material may be covalendy modified with a cell-binding peptide prior to formation of a device described herein (e.g., a hydrogel capsule described herein) using methods known in the art, see, e.g., Jeon 0, et al., Tissue Eng Part A. 16:2915-2925 (2010) and Rowley, J. A. et al., Biomaterials 20:45-53 (1999).

For example, in the case of alginate, an alginate solution (1%, w/v) is prepared with 50 mM of 2-(N-morpholino)-ethanesulfonic acid hydrate buffer solution containing 0.5M NaCl at pH 6.5, and sequentially mixed with N-hydroxysuccinimide and 1-ethyl-3-[3-(dimethylamino)propyl]carbodiimide (EDC). The molar ratio of N-hydroxysuccinimide to EDC is 0.5:1.0. The peptide of interest is added to the alginate solution. The amounts of peptide and coupling reagent added depends on the desired concentration of the peptide bound to the alginate, e.g., peptide conjugation density. By increasing the amount of peptide and coupling reagent, higher conjugation density can be obtained. After reacting for 24 h, the reaction is purified by dialysis against ultrapure deionized water (diH2O) (MWCO 3500) for 3 days, treated with activated charcoal for 30 min, filtered (0.22 mm filter), and concentrated to the desired viscosity.

The conjugation density of a peptide-modified alginate is measured by combustion analysis for percent nitrogen. The sample is prepared by dialyzing a solution of the chemically modified alginate against water (10,000 MWCO membrane) for 24 hours, replacing the water twice followed by lyophilization to a constant weight.

### Example 4: Preparation of Exemplary Alginate Solutions for Making Hydrogel Capsules

70:30 mixture of chemically-modified and unmodified alginate. A low molecular weight alginate (PRONOVA™ VLVG alginate, NovaMatrix, Sandvika, Norway, cat. #4200506, approximate molecular weight <75 kDa; G:M ratio ≥1.5) is chemically modified with Compound 101 to produce chemically modified low molecular weight alginate (CM-LMW-Alg-101) solution with a viscosity of 25 cp to 35 cP and a conjugation density of 5.1% to 8% N, as determined by combustion analysis for percent nitrogen. A solution of high molecular weight unmodified alginate (U-HMW-Alg) is prepared by dissolving unmodified alginate (PRONOVA™ SLG100, NovaMatrix, Sandvika, Norway, cat. #4202106, approximate molecular weight of 150 kDa-250 kDa) at 3% weight to volume in 0.9% saline. The CM-LMW-Alg solution is blended with the U-HMW-Alg solution at a volume ratio of 70% CM-LMW-Alg to 30% U-HMW-Alg (referred to herein as a 70:30 CM-Alg:UM-Alg solution).

Unmodified alginate solution. An unmodified medium molecular weight alginate (SLG20, NovaMatrix, Sandvika, Norway, cat. #4202006, approximate molecular weight of 75-150 kDa), is dissolved at 1.4% weight to volume in 0.9% saline to prepare a U-MMW-Alg solution.

Unmodified alginate solution. An unmodified medium molecular weight alginate (SLG20, NovaMatrix, Sandvika, Norway, cat. #4202006, approximate molecular weight of 75-150 kDa), is dissolved at 1.4% weight to volume in 0.9% saline to prepare a U-MMW-Alg solution.

Alginate Solution Comprising Cell Binding Sites. A solution of SLG20 alginate is modified with a peptide consisting of GRGDSP as described above and concentrated to a viscosity of about 100 cP. The amount of the peptide and coupling reagent used are selected to achieve a target peptide conjugation density of about 0.2 to 0.3, as measured by combustion analysis.

### Example 5: Formation of Exemplary Two-Compartment Hydrogel Capsules

Suspensions of genetically modified cells as single cells are encapsulated in two-compartment hydrogel capsules according to the protocols described below.

Immediately before encapsulation, a desired volume of a composition comprising the cells (e.g., from a culture of the cells as described in Example 1) are centrifuged at 1,400 r.p.m. for 1 min and washed with calcium-free Krebs-Henseleit (KH) Buffer (4.7 mM KCl, 25 mM HEPES, 1.2 mM KH₂PO₄, 1.2 mM MgSO₄×7 H₂O, 135 mM NaCl, pH ˜7.4, ˜290 mOsm). After washing, the cells are centrifuged again and all of the supernatant is aspirated. The cell pellet is resuspended in the GRGDSP-modified alginate solution described in Example 3 at a desired cell density (e.g., about 50 to 150 million suspended single cells per ml alginate solution).

Prior to fabricating hydrogel capsules, buffers and alginate solutions are sterilized by filtration through a 0.2-μm filter using aseptic processes.

To prepare two-compartment hydrogel millicapsules of about 1.5 mm diameter, an electrostatic droplet generator is set up as follows: an ES series 0-100-kV, 20-watt high-voltage power generator (EQ series, Matsusada, NC, USA) is connected to the top and bottom of a coaxial needle (inner lumen of 22G, outer lumen of 18G, Rame-Hart Instrument Co., Succasunna, NJ, USA). The inner lumen is attached to a first 5-ml Luer-lock syringe (BD, NJ, USA), which is connected to a syringe pump (Pump 11 Pico Plus, Harvard Apparatus, Holliston, MA, USA) that is oriented vertically. The outer lumen is connected via a luer coupling to a second 5-ml Luer-lock syringe which is connected to a second syringe pump (Pump 11 Pico Plus) that is oriented horizontally. A first alginate solution containing the genetically modified cells (as single cells) suspended in a GRGDSP-modified alginate solution is placed in the first syringe and a cell-free alginate solution comprising a mixture of a chemically-modified alginate and unmodified alginate is placed in the second syringe. The two syringe pumps move the first and second alginate solutions from the syringes through both lumens of the coaxial needle and single droplets containing both alginate solutions are extruded from the needle into a glass dish containing a cross-linking solution. The settings of each Pico Plus syringe pump are 12.06 mm diameter and the flow rates of each pump are adjusted to achieve a flow rate ratio of 1:1 for the two alginate solutions. Thus, with the total flow rate set at 10 ml/h, the flow rate for each alginate solution is about 5 mL/h. Control (empty) capsules are prepared in the same manner except that the alginate solution used for the inner compartment is a cell-free solution.

After extrusion of the desired volumes of alginate solutions, the alginate droplets are crosslinked for five minutes in a cross-linking solution which contain 25 mM HEPES buffer, 20 mM BaCl₂, 0.2M mannitol and 0.01% of poloxamer 188. Capsules that fall to the bottom of the crosslinking vessel are collected by pipetting into a conical tube. After the capsules settle in the tube, the crosslinking buffer is removed, and capsules are washed four times in HEPES buffer, two times in 0.9% saline, and two times in culture media and stored in an incubator at 37° C.

### EQUIVALENTS AND SCOPE

This application refers to various issued patents, published patent applications, journal articles, and other publications, all of which are incorporated herein by reference in their entirety. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present disclosure, as defined in the following claims.

1. A genetically modified cell comprising at least one exogenous transcription unit inserted into at least one genomic site, wherein the cell is derived from a human cell and the genomic insertion site (GIS) is selected from the group consisting of:
(a) a Chr 1 GIS located between two contiguous or non-contiguous nucleotide positions x and y within SEQ ID NO:1 or a human genomic nucleotide sequence that is at least 90%, 95%, 98%, or 99% identical to SEQ ID NO:1;
(b) a first Chr 2 GIS located between two contiguous or non-contiguous nucleotide positions x and y within SEQ ID NO:2 or a human genomic nucleotide sequence that is at least 90%, 95%, 98%, or 99% identical to SEQ ID NO:2;
(c) a second Chr 2 GIS located between two contiguous or non-contiguous nucleotide positions x and y within SEQ ID NO:3 or a human genomic nucleotide sequence that is at least 90%, 95%, 98%, or 99% identical to SEQ ID NO:3;
(d) a first Chr 3 GIS located between two contiguous or non-contiguous nucleotide positions x and y within SEQ ID NO:4 or a human genomic nucleotide sequence that is at least 90%, 95%, 98%, or 99% identical to SEQ ID NO:4;
(e) a second Chr 3 GIS located between two contiguous or non-contiguous nucleotide positions x and y within SEQ ID NO:5 or a human genomic nucleotide sequence that is at least 90%, 95%, 98%, or 99% identical to SEQ ID NO:5;
(f) a third Chr 3 GIS located between two contiguous or non-contiguous nucleotide positions x and y within SEQ ID NO:6 or a human genomic nucleotide sequence that is at least 90%, 95%, 98%, or 99% identical to SEQ ID NO:6;
(g) a first Chr 4 GIS located between two contiguous or non-contiguous nucleotide positions x and y within SEQ ID NO:7 or a human genomic nucleotide sequence that is at least 90%, 95%, 98%, or 99% identical to SEQ ID NO:7;
(h) a second Chr 4 GIS located between two contiguous or non-contiguous nucleotide positions x and y within SEQ ID NO:8 or a human genomic nucleotide sequence that is at least 90%, 95%, 98%, or 99% identical to SEQ ID NO:8;
(i) a first Chr 5 GIS located between two contiguous or non-contiguous nucleotide positions x and y within SEQ ID NO:9 or a human genomic nucleotide sequence that is at least 90%, 95%, 98%, or 99% identical to SEQ ID NO:9;
(j) a second Chr 5 GIS located between two contiguous or non-contiguous nucleotide positions x and y within SEQ ID NO:10 or a human genomic nucleotide sequence that is at least 90%, 95%, 98%, or 99% identical to SEQ ID NO:10;
(k) a first Chr 6 GIS located between two contiguous or non-contiguous nucleotide positions x and y within SEQ ID NO:11 or a human genomic nucleotide sequence that is at least 90%, 95%, 98%, or 99% identical to SEQ ID NO: 11;
(l) a second Chr 6 GIS located between two contiguous or non-contiguous nucleotide positions x and y within SEQ ID NO:12 or a human genomic nucleotide sequence that is at least 90%, 95%, 98%, or 99% identical to SEQ ID NO:12;
(m) a third Chr 6 GIS located between two contiguous or non-contiguous nucleotide positions x and y within SEQ ID NO:13 or of a human genomic nucleotide sequence that is at least 90%, 95%, 98%, or 99% identical to SEQ ID NO: 13;
(n) a fourth Chr 6 GIS located between two contiguous or non-contiguous nucleotide positions x and y within SEQ ID NO:14 or a human genomic nucleotide sequence that is at least 90%, 95%, 98%, or 99% identical to SEQ ID NO: 14;
(o) a fifth Chr 6 GIS located between two contiguous or non-contiguous nucleotide positions x and y within SEQ ID NO:15 or a human genomic nucleotide sequence that is at least 90%, 95%, 98%, or 99% identical to SEQ ID NO: 15;
(p) a first Chr 7 GIS located between two contiguous or non-contiguous nucleotide positions x and y within SEQ ID NO:16 or a human genomic nucleotide sequence that is at least 90%, 95%, 98%, or 99% identical to SEQ ID NO: 16;
(q) a second Chr 7 GIS located between two contiguous or non-contiguous nucleotide positions x and y within SEQ ID NO:17 or a human genomic nucleotide sequence that is at least 90%, 95%, 98%, or 99% identical to SEQ ID NO:17;
(r) a Chr 8 GIS located between two contiguous or non-contiguous nucleotide positions x and y within SEQ ID NO:18 or a human genomic nucleotide sequence that is at least 90%, 95%, 98%, or 99% identical to SEQ ID NO:18;
(s) a first Chr 9 GIS located between two contiguous or non-contiguous nucleotide positions x and y within SEQ ID NO:19 or a human genomic nucleotide sequence that is at least 90%, 95%, 98%, or 99% identical to SEQ ID NO: 19;
(t) a second Chr 9 GIS located between two contiguous or non-contiguous nucleotide positions x and y within SEQ ID NO:20 or a human genomic nucleotide sequence that is at least 90%, 95%, 98%, or 99% identical to SEQ ID NO:20;
(u) a Chr 10 GIS located between two contiguous or non-contiguous nucleotide positions x and y within SEQ ID NO:21 or a human genomic nucleotide sequence that is at least 90%, 95%, 98%, or 99% identical to SEQ ID NO:21;
(v) a Chr 13 GIS located between two contiguous or non-contiguous nucleotide positions x and y within SEQ ID NO:22 or a human genomic nucleotide sequence that is at least 90%, 95%, 98%, or 99% identical to SEQ ID NO:22; and
(w) a Chr 14 GIS located between two contiguous or non-contiguous nucleotide positions x and y within SEQ ID NO:23 or a human genomic nucleotide sequence that is at least 90%, 95%, 98%, or 99% identical to SEQ ID NO:23;
optionally wherein, for each GIS defined in (a) to (w), the x and y positions are independently selected from the group consisting of:
x and y are 100 and 1700;
x and y are 200 and 1600;
x and y are 400 and 1400;
x and y are 800 and 1000;
x and y are 850 and 950;
x and y are 875 and 925;
x and y are 890 and 910; and
x and y are 898 and 903.

2. The genetically modified cell of claim 1, wherein for each GIS defined in (a) to (w), the x and y positions are 890 and 910.

3. The genetically modified cell of claim 1, wherein for each GIS defined in (a) to (w), the x and y positions are 898 and 903.

4. The genetically modified cell of any one of claims 1 to 3, wherein the exogenous transcription unit is inserted into each of at least five genomic insertion sites, which are the Chr 1 GIS, the first Chr 3 GIS, the fourth Chr 6 GIS, the first Chr 7 GIS and the second Chr 9 GIS.

5. The genetically modified cell of claim 1, wherein the exogenous transcription unit is inserted into each of at least 18 genomic insertion sites, which are the first and second Chr 2 GIS, the second and third Chr 3 GIS, the first and second Chr 4 GIS, the first and second Chr 5 GIS, the first, second, third and fifth Chr 6 GIS, the second Chr 7 GIS, the Chr 8 GIS, the first Chr 9 GIS, the Chr 10 GIS, the Chr 13 GIS and the Chr 14 GIS.

6. The genetically modified cell of claim 1, which is derived from the ARPE-19 cell line.

7. The genetically modified cell of claim 1, wherein the exogenous transcription unit comprises a promoter sequence operably linked to a coding sequence for a polypeptide, optionally wherein the polypeptide is a GLA protein.

8. The genetically modified cell of claim 7, wherein the polypeptide is a GLA protein and the exogenous transcription unit comprises SEQ ID NO: 29, SEQ ID NO:30, or SEQ ID NO:32.

9. The genetically modified cell of claim 4 or 5, wherein:
(a) the cell is derived from the ARPE-19 cell line;
(b) the exogenous transcription unit comprises, consists of, or consists essentially of SEQ ID NO:33; and
(c) for each GIS the x and y positions are 890 and 910.

10. The genetically modified cell of claim 5, wherein:
(a) the cell is derived from the ARPE-19 cell line;
(b) the transcription unit consists of SEQ ID NO:33; and
(c) for each GIS the x and y positions are 898 and 903.

11. A composition comprising a plurality of genetically modified cells, wherein each cell in the plurality is the genetically modified cell of claim 1, optionally wherein the composition further comprises a culture media or a cryopreservation medium.

12. The composition of claim 11, wherein the plurality of cells is obtained by culturing a monoclonal cell line.

13. The composition of claim 11 or 12, wherein each cell in the plurality is the genetically modified cell of claim 9 or 10.

14. An implantable device which comprises at least one cell-containing compartment comprising a genetically modified cell or a plurality of the genetically modified cell and at least one means for mitigating the foreign body response (FBR) when the device is implanted into the subject, wherein the genetically modified cell or each cell in the plurality is the genetically modified cell of claim 1.

15. The device of claim 14, wherein the at least one cell-containing compartment comprises a polymer composition which encapsulates the plurality of genetically modified cells, wherein the polymer composition comprises an alginate covalently modified with a peptide, wherein the peptide consists essentially of or consists of GRGDSP, GGRGDSP or GGGRGDSP.

16. The device of claim 14 or 15, wherein the cell-containing compartment comprises an alginate hydrogel and is surrounded by a barrier compartment, which comprises an alginate chemically modified with Compound 101.

17. The device of claim 14, which is a hydrogel capsule of about 0.75 mm to about 2 mm in diameter.

18. The device of claim 17, wherein the cell-containing compartment comprises a plurality of the genetically modified cell of claim 9 or 10.

19. A preparation of devices, wherein each device in the preparation is a device of claim 14.

20. A method of treating a human subject for Fabry disease, comprising:
(a) providing a composition comprising a plurality of genetically modified cells expressing a GLA protein; and
(b) disposing the composition in the body of the subject;
wherein each cell in the plurality is the genetically modified cell of claim 8.

21. The method of claim 21, wherein the composition comprises the preparation of devices of claim 19, and optionally the disposing step comprises placing the composition into the intraperitoneal space or into the greater sac of the peritoneal cavity.


SEQUENCE LISTING (40 sequences)

SEQ ID NO: 1 - DNA, 1800 bp, Homo sapiens
cgactcttcagataaaaccaagcataagggttgggagagggaagcaagtatgtgaaggaaatagagaagttatcagcacaataggaagggaattatgaagttgactgcagtgtgaggatcagctgtggagaaagccactaccaatgcgaaggcgtgaaattaaagtggggagcatggaggaaaagagaaaggctgtctgcagctcagataaaatccttgatgtatcactgttgaaaatggagtggacctcagtgatttgaggaaggcaaagatcacacattgaagtttaacagtaaagaaaggaagacactggatgatagctgggtggaggaaaagtcaacagaacaagcgtgaagaattcagtgaggtcccaaaggagacacaaacaggtcacaggggatagatgtgaaaaaaaagaataaggatatctttcctgaaataggaagaaatggagaaagagatattttgaggaatgcacctccattttcacacaaagaagaagacaagatcatctattatgaaagggaaaggtgcatttggaaaccaaaagaaagcattcaagatatagaactgattcttggaaacaatttgctctttgtcaggaagccagcaatgataaactctatcactaattttagtatcaattccttaaaaatgattttcaatatatgagaaaagttggaaagcgattaggggagtgtgtgagggtacagatgaaacaagaaaggaaatgaactgatattacttctattctttctacgttatgtttgaaatctcccaaaacctttttttcaattttaaggaacgcaattaattcctcatcgttcatatttctaagatcaaacaaagacattgcttggtaaataagaatacggaatgcacctatttcccttacttcttaatgacaaatgccttttccatctttattttgttgaatagaacagctctcaaaaactatgaaatattcaaaatgaatgccagggatttctgtcttctgaattaattcagatcttgcaaaacataacttatgatgtcaatacaatagggcaactgagagtcaaaaacaactatttaagtgaaacaaatctcccagcattgtcaaaatcatgtcttagtttaaaaatatataaaaaatattctttacttacataagaaaaacaatcccagaactttggaagaaattgagggaatttatgaagaatcaatataataatccattctataaaatattttatggatgcttgattgttggtgaaaccagcctggaaaatcctgtcaataattccattcaagaaattctgagaaaaaaagttcacaattaaccagttacagttattcattttaatttaaaaatagtctccatttaaaataaaatagcttaaagtttttatcataagtaatttattggatctacaactctccgagcaaagactctaaaaactactgattaaaatataatgatgaagtaccttcattcagtactttataatttacctggacatcagttctatctataaggaaggttatccatcacctgtatttttacagggaaaggaaaagactcagaaaagctgaataaccctcccaaagtcacatacctagtaagtggaagaattcaggttcaaatcaagatctgccaagatgccagggcttaaaccttgtaacaagggatggtacagagactgaagtcagcaggtgggaaacagcatcccagaggggcaggtcttgggccactgagccctctgttcaggctcgtcgtcctcctgccctcatggtctgtgttgccccacaactaccgca

SEQ ID NO: 2 - DNA, 1800 bp, Homo sapiens
tcgagaccagcctgcccaacgtggtgaaacccgtctctactaaaaatacaaaaaaattagctgggcatggtggcgggggcctgtaatctcagctactcgggttaggagaatgaacccaggaggcagaggttgcagtgagccgagatcacgccactgtactccagcctgggcaacaagagtgaaactctgtctcaaaaaagaaaaaaaaaaggggccaaatgattacgtgaactgatcgcaccacttaatcttgtgtctcacctggacagagctagtcatagtgctcagagcaaacaccgttgcacagtctttgatagttgactggctatcaaaggcaccctgggtatccataagcagcacagcaacctaggaatttgagagtttaaaatatttagtaaaacagttgccaaaaatgactataaaaaaaatcaaatgataaatttgaaaagcagtaattaaatcaagaaaagtacttatatctcatacatttgacataataatccgtatgttaaagttatcaaacattaatagttctgtgttgttttgatttttaatactggaaaatagcccaagaaagaatacagggtagaaaagtataaacagaattttactttagttccattaggtctgtcaatcacaaatacttcattccaaacttgtatgcctgttgtttctctttcacagccacctcgccatgtaaagcctgtcaatggttcattgtttccaccaatccaactttgagaatcctgttaaagtcaaggataaatgtaaaatacaacacaattaagaaataaaagaaaccaaaaaatcacccattctttattttacagatggggaagctaaacccggaaagacaaaaaaaacactgtgtaacaaacaagtagttacaaagcagtccttaaatcccatgtggggttctttttcttatagagacgtggtctcctccaaactcgaatcttctcaaatcagctcacactactactctctaggcttctaaataagtggggttatactcactttcccccttttctgaaagatatcctggctgggcatgacagctcatgcctgtaatcccagcactttgggaggctgaagtgggcatatcacttgaggccaggaatttgagaccagcctggccaacgtggcaaaacgccatctttacaaaaagtacaaaaattagccaggcgtggcggcacactcctgtagtcccggctactcagcaggctgaggtaggaggatcacctgaacctgggaggttgaggctacagtgagctgagattgtgccactatactccagcctgggtgacagagtgagatcctgcctcaaaaacaaaaaaaaaaaagagagagagagagagagacagagaaatatttcctaaaatgttaaaaatcataaaaaactcggcaccaattattttaacatgttgttacacagataagcaaaactaagttccaggtagaatgactgtcaaaacagcactgctttctcagaatagcttacagtaagttttcatactgccttcatttcctctttaaaaaataatcaattttaggctgggtgcagtggctcatgcctgtaaccctagcactttgggaggacgaggcaggtggctcacctgaggtcaggagttcgagaccagcctggccaacatggcgaaactccatctctactaaaaatacaaaaattagccaggtgttgtggcatatgcctataatcccagctactcaggaggctcaggcaagagaatcacttgaacccgtgggcagaggttgcagtgagcagagatcgcgccactgcac

SEQ ID NO: 3 - DNA, 1800 bp, Homo sapiens
tgttgcaaggtatatctgatcaaaggggaacccaggcatcaggaggaggctagggacaggggaccacgccattgcatggtgacactcagcagacatctgcctctcaaataaaccagaatcctcaatctgtgttgagggtaaaagctggaaactaggagagagagacgctgcctgggtaaagagacggaaggaactcagcctaggtccaagggggagctcattccacctagaaagagaatcctgatctctctggaaaaaaaggagagatgttgagctacaaggaggcaaggctcacagagccagatgaaaacatcctgcttagagcacatggaatgtctctgttacatttctgcggtcactgaactataaactaggctcctgcaatatatatctgcacttggtcacctgaatatataaaggtgtttaaaaagcaagtctgcaagccatttctctcttttttttttaactgactctcttccaaaaaagtaactaaatgacacattactatgtttccacataatctcttatcagatttgagcagatttgtcatagtcctagacaggtgttagtatctttgtttatcccagccttccacaccttgcaagatcattcagccttccttctagaaattttattcccttctttgatacactggattgactgaaaacaggttttgcatatacttcaagagaataaaaatattttttgcattaaaaatagaatcaccacaggcacagagatagttagacatttaaacaaagggtatatcaaatcttctattcttctagctaatctattttcagtactgccagaaacacctaacaaactacagtctcacaaaatcagaatctgtggtactagtatcatgtggtgatgattaaccttgtcaacttattttttaaataatcctcatcgtttataccattgtagtaaagggttcccctctcccatgcagcaagtccagaatgaaagtgggttgttcccatggcaacagaatacaaggcagagaactccagagaaggggggcgggtgcatctctacactcaaggccatagctgcaagaaagccccaggctccaggatggtggcagcaaagagatgcaacctgccatgacatcacactgccagatggcctgctgttgacatagggttcccccccaactaattatttgcataaatctggatcattcattcaaagaaaaataaaggattcactcaagtatacattgacgtacttattagacagtttttctctttgaacaacttagtattacaaacattccccaaacaccaagcacatagttgccttacacatggaagctgtttaagaagcatgtatttcacgatatattggaccccttcctgaaccaggtattgctcaagatccagaggagagttctctctctccagagagttcactgtttagtgggtggacaagcatgtataaagatccatagaaccttacatatcagcttaatgatagacttgcccagggtatcacaggggtgagaattcaggagaggagaccacaaccctggagaaatacataggaggggagaaaggagacaacgtacgtgtcacatgaccccgaggatgaatgaggagctattgatgctccattgtttgctctgtctgcttgctttgagattgttggcggagcaaacagcacaagcaaagacccaaaggcaaaaagtagcacagtctaagtaggggaagcatatgcagtttgcttgtgttgaacacaaatctgtaaggcagggactggagggagctgaagctagagacacagggagctaagatg

SEQ ID NO: 4 - DNA, 1800 bp, Homo sapiens
gcaggggtagtgcacttgccattagtccttttaagttgcaattcttcccaccagctaaagagaaatatataaattatttctagctaggtggcaaaactcccccagccatcttgtcaaatgattgcatgggtgcaaagatctataaattcctccataggttgcatatcctcaccattcagggaggccccaagttgtcaatgaatagatggccaggcccaatactggaccagccagtggtgaaagggctgctgtttggagaggtggagctagtgctgagtataggaagaccatgtagaaataccaaagtcagacccattggaggtttgagcagattgtgagatttttcaggtacaccagggaggcaagatagtacatctctagtgctggacttaaactgaaggcagtccttctttaaattgttttggtgccctctgggcattaaacagatgcagtcttccacttatggccaatagttagagccacaaagagtctatttcaagtttcaggagatgaatggcatctctgctgctctttgagagactttaaggtatttttcccctgaattgtgattgggttcccaggcagggttcagaacattgtgtttattgtctgccgtattatcttttaagcaatgctgtactgcctgatggtatggaagaggtaacatagatgtttgtaaacatacagtcatttgatggctaaggaagttttgccacccagctagaagtaacttatgtattgttagctagaagaatttcaacctgaaattgacccaatttttcactctttaaaacagagcagttgctagaatagtgggtaggtttctagatttaacctggtttatcttacattttagttggtcaaaccttaatggctttttcaaagacttagagaaagtttaaaagcaatagtgactaaatagatggtggagcagaagggagcatggtttcatccacgagcctgagttttctttttgaattttcctctactaaattatatctttagttttggaatcatacgtcacatcaaattttagattagagatggtaaactgtttggcctgtgagccagatacggcttgcaggtatgtattttttggcctgcccagtttgttaaattctgaataaagccatcatttaaaaaccacaagttttaggcggggcgtggtggctcacagctgtaatcctagcactttgggaggccgaggggggcagatcacctgaggtcaggagtttgagaccagcctggccaacatggtgaaaccccgtctctactaaaaatgcaaaaattagtcgggcatggtggcacatgcctgtaatcccagttactcgggaggctgaggcaggagagtgacttgaacccgggaagcaaaggttgcagtgaaccaagatggcgccactgcactccagcctgggcgacagagcgagactccatctcaaaaaaaaaatcacaagagggtctgaattttcccatttccaactttgaaaaatcagatctgggaactccagacatgcatctcttaatagtgatggttagctagagccaagaagcaaagtttatagataatgcacttgatataaaatttaaaggtattacgaaatactctgggctgggcatggtggctcatgcctctaatcctagcactttgggaggccaaggctggtggagtgcctgagctcaggagtttgagaccagcctgggcaacacggcgaaacctcatctctactaaaaatacaaaatctagcccggtgtggcaatgtgcgcctgtagtcccagctactctgggatgaggcaggaaaa

SEQ ID NO: 5 - DNA, 1800 bp, Homo sapiens; identical to SEQ ID NO: 4

SEQ ID NO: 6 - DNA, 1800 bp, Homo sapiens
agttagacttaaaaaaagatattaaaaagatttgaaaacctgatggagaaaatctgtaaagctgattaaatattcatttaatagttgttcctgaagatagacattgaagcaacaaaacagaaaaaattatttaaaattacaattatcaaaatattctggaaaaacaaaaattgattctattgaaaattcatactctccttgtacatggagtcaataccaagacagattagactataaagataaggaaaaattgccttcagaaattcagaaccacacgtcctccatccttactccataccaccaacacatatacataaatcaattaactaattacaaaaatgaaagtcatcctaaatttctaaacaacaatgttttaagctacaaaacaattaagcaacatttttaaagtattgaaggaaaccatatgtgagccaaggtttttatacctggtcaaacatcctttggtacaaaggctataaagagttataaacaggcactacttgaaaaaataccatttccataaaccttttctagggaacttatcacagaaagagattcagaaaactgtaacatctggaaagctctttcatatggtctagtggcctggcagtaagcatttaaggaccagaaaatgaaagatgataaaatgcaatgtgcatattacaggccctgatgttggagaactagtacaactaattactaattgggatggaaaggggtaaatagaaattagaatacactcgctgattgacttactggtattaggaagggaaagatataatttaaaacaaaaattaagagcttgaagcttaagtatgttttgctctacaaacaacactaagattatacttatatctagcaagagaggaaaagaagcagtttctggctgacaataatattaataatagaaagaaactgtttcagaacagagtatggttaaatgcattatagataggtattagtgtaaagctaaccattagaaagacttcaaacgttcccaaatagcaaaggaaataatgggggagaagggcaaacaactaaaataatggaacatgcacatgcattcacatttatataacataatactttattatagaactgagaccaaatatatcagttgtatcaatgcatgttagtggacttacataagaaaaagattttcagattggctaagaaagcaaaattcaactttatgctgtatgcaaaagatatacctaaaataaagtgaattgaaatgggtaaagataaaagcatgaacaaaaatacaccaggcaaaggcaagaaagaagaaagcaggggccatgaccttgatctaaaacaaggtagaattcaggccaagaagaattaaacgatacaaagtaggacacactataatgctaaagagtgcgattcacagtgaagatattaatatctattcaccaaatatcagtagcaactttcataaagcagaaactacaggcgatgaaaggagaaatagacacatattaaaaataggaaactttagcacacttatctcagatctagacagatcaaggggaaaaaatgttagtaaggacatagaaggcctaaaaaccataagatagagtctataaatatataagcactttatcctgatagcagaaaatatacctttttttcaagtatacataaatccttattaaaattaactatatattaggccataaataactcaaaaatagaaatacaaacaacattaaaaggtaagtcctcaattagtaataataataaaaaatgcacttctcaccaggaaacttttttaaaaagtaagctcatatcaaa

SEQ ID NO: 7 - DNA, 1800 bp, Homo sapiens
aaactgagaaacataagtacagataagtacagattgataacaatgaaatgacacttcaaaaagataatataaacaatattagcctttatcctcttttttaaatcacatgaaagcactcagcacagtgagaaacgtgttgggcaatgtttaggggtatgcacagaaagaaaaatctatatggctgcaccacactagtagctgtaagtctctctggagaaaaggctagaattggtgaaggaggactgtctcaagatttagtcagacaagtagaccactagagaaagaataaagtatatatagaaaatttctacatatatatacatacacaaacacatatatgaattttatttttacaagaatattacctggcagttgcgaatgaaagatggctgaaagatggggaacagcaaaaacattctggaatctacagatacagccagaagcagaaacataaaggagagcaaattctgggaaatgtaatctagcctaatcgcattacaaatgtgacacattacaaagcctccacaggggctttaatttatattattaaataaatttttggagggaaaagttattttaaaaactcaaaatgacttttttagagtttttaaaaataaatcaaatctaaaccattatactatgtcatataaacaaaaaatcaattatttttaaggctattatgacactggtaacattcacgctaatgatttatgtaaacaatcaaaagcaaaatatatacaagctggatgctttgagaatatctgaagaatcgagtgagttttctcagccgagggatggtgtaatagtggcatgccctttacaatgtccagtattcttacatagtcaaggatgaggttctatgcgtcttgcacagatgcaaaagttctgaagtgtaacattaaacataacatctttttccccttaattaaccagagagaattctcaacattcagcaaactgctcatcgcatttttgctagattccttttctactaattctattcttttaagtcataaacttcttaaagtaaatgaaagtaaaaaaatagcaattgtcaaaaagggagagaccctatgtttcctccagccattttctataaaatatcagctttttgtggaacttgaaataaaatattggcatatctgaaaaccttacttaattgtgaccttgataatatggtgtttcatgcaacccaaataaatgttcagactgcattaaggtggggcagtaggaaaaacaactccaataatgagatgttactgtagctctaggaatcataagtctcttgtacatttgtatagcactgacataccagcctgaagaactttgaaaatgaggttttccaagccgtggatcagcacacattggcttttgccaagttgtaaagtgttgatctaattctttacatttaagcctgtccaccttatggctgacagccaatgaaatcaggccacgggccttccattcatggagctttagccctgtaacaggcagtgaagtgctaagcattttatatacattatttaacttcaccttcacagctaaccagtgaggtaattattatctctgttttgctaaagaaaagaagagttgagtaacttttccaagttatgtctgattccaagcacatgtgtgactattacattatcctagattcatctggttctgttcatgatgacagcccagaatcttccttgccttagtaatagcagccaatatttatggagcaccaacctgtaacagatgctgggctaaatgatttatgccattttaatcatttaatcttcacagtctgcctat

SEQ ID NO: 8 - DNA, 1800 bp, Homo sapiens
gtggacacccctcccctcaccaagctcgagcatcccaggtcaatttcagactgtgctagcgagaatttcaagccagtggatcttagtttgttgggctccttgggggtggaatccactgagctagaccacttggctccctggcttcagccccctttccagggtagtgaatggttctgtctcactggtgttccaggagccactggagtatgaaaaaaagaaaaaaaaacaactcctacagctagcttggtgtctgcccaaatggctggctgcccgattatgtgcttgaaacccagggcccccggtggtgcaggcacccaagggaatctcctgatctgtgggttgtgaagaccacaggaaaagcatagtgtctgggccagaatgcactgtgcctcatggcacagtcactcatggcttcccttggctaggggaaggagttcccgaccccttgcacttcccaggtgaggtgatgccccaccctgcttctgctcgccctccatgggctgcacccactgtctaaccagccccagtgagataaaccggctacctcagttggaaatgcagaaatcacccaccttctgtgttgatctcgctgggagctgcagactggagctgttcctattcggccatcttgccacccgctgatgtacccattgtattctaaatattttgtaatattaacacatttaaccatcacaacaactttatcagttctgttcttattcccatagtgcaggtgaggaacttgaggcatgaagacaccaaattgcagagaatttaaacccaggcaacctgactttgccactcttgtaaagagtcaatatagtttagtgcctcagtaaaaaatgaaatgtttgattatgaatgaatttatcaagtgaggtttgaatatgaccaaaatttaaggtaactaatcctgtgtcttatactgtgactattgagtttataattagcaatataaaattaaggatattgactctgtaagtatttgttccataaaagcctaatgatcagattagatacttaacctgcatttcaagtacagatttaaaataaacaagggaacagataatttacagccagttaatatgaagtgggtcatatgatgtcctagttattatagacctaaggtgattggacacttggtatgcaaaggagtaccttctatggaagaggaatcatcaaatatgagaaactgtgccatttcagaaaatcagattaaatttttgaaaaaattctatggaaatttttggaaatgattaagttttgtccatctaaaatggaaaatactgtttttagtgatatagttttattcttttgtgttcattatattaagatttattttcatgaaattcattcaaattattgttttcaattaatctactgagacccagtgtttgtcaaaaagaggcttaatttttatgtctgcattttagacagatttataacatgatctataaaccatagatctctcacattactcatttgaagaactgcatgattttaacacttataaatgtagagattaggaagaaagaaagaaatccattaaatccagtgatcactatttattagagaccttttgctgcttaaacaaaacctataagacacacccactatgtaaccataaaaattaaatataaaaagcaaaatgaatgaaaatgtcaacaaattttaaatctttaaaagcgtactattcctcagttctatattagctgttaacacgtaaaagtagcaaaatagttatgtattagtatttaaactcatgatataaagatatatatcatatttgattacattacc

SEQ ID NO: 9 - DNA, 1800 bp, Homo sapiens
cccctatgtaatctcatcatttccaatgtttaattccatgatatactaatgagtgcccagtttataacttcagctcatacttctcagagatccatatttacatattcaaccacctagttgacgttcttcacctatctcagaaatttctcagatttaacatttttaacattttcaaaatagaaaccttggtttctgattcccaaacctgcctttgctcctccacatatcttccatatacatccacccagcacccggcaccaaaccatgggatcacgttttgttctccatttccctccacacccagcatccgatctatcagcaagtcctttcaagtcaacctgaaacatgtgtcatgaatctgcctctctcccccactgccatgccagtccaaaccaccatcatttctcacctgctccactttaacacttcccaacttgtgtctctacttgcactgtgccccccaacacaactcagtccactctccacacagcaggactgtaagttataacacaaatcagatcaacatcaaatataaattgaatcgtgccattcccctgcttcaaacttttaatggctttctagttcatgtgaataaaatatagtgtctttaccataagctcagcatgatctagtctccacctatctcccccaccttatctcatgccaagctttccccttaatcagtgatttctagccacactgatcttctcttagttccccaaacacatccaactcattactgattcacgatctttgtatgcagtagtttttcctacctgtagcccttcctccttaaattagaatgccatatcctcaagaggccttctcatagaaccctatctgaagacaatatccctcacactttccttactttctgcattggctcattgtttctttattaagcttgtcataaatttaattgtttttattgaacaaggttctgataaaacttgtttcttctgtgtctttcagtaaaatgtgagccccatgaaggcaagaatgaatcatgtctaacttgttccccattattgtatccacactgcttagcacggagggaatgaatgaagtaatgaagatcagataatattatcaaaagttttcataagcctgttcctcaagctgcaataccagtttgggacatactttagccatctattcctgtggaaacttctaaatagctgtccttggtagatgcaagtctcttacacaccatagtcttactgacgaaaattagataccctggaaaattactaaagttgaaatgacacatgatttctaatttgacttttccagggaaacagtgttctcaaatagcttttcatagagctaaccattagcccaacatttaactaactgaaacagaataactattgtctaaaccaggatacttctaagagtaaaaaggtttacttttcactttaaataatttaggaaccataggtgtaaattaagactattccaggcaaatcaggacaagccctaacatttaaaaatattctctttgactatgtggtttattcttcctagagcttgcaattaaacaagttaatatactggcaaatgataaaattatattcagataatatggcctaataacctattccacaaagttcctgcctttttctccacaatgatgatctattttgtatgacaacaaatcaccaggtaaggcaacacatttttaaatatgttgggatccaaaagagtctctttagggagaatttgataggcatctttgagggaatatttgaaagagatttgcagtcccttgacaaataactttttgataaatcaataaat

SEQ ID NO: 10 - DNA, 1800 bp, Homo sapiens
gattttgagaaaaaaattgttgggctctaacaaactctattactcattgccctgaagtgcagcggtccccaacctttttgtctccagggaccagtttcatggaagtcaatttttccatggactggtagtgggaagtgggggggcactggggatgattcaagtgcattacatttattgagtactttatttctgttattattacattataatctataaagaaataattatacaactcaccgtaatatacaataagtgggagccctgagcttgttttcctgaaaccagatggtcccatctgggagagatgggagatattactgatcaccaggcattacagtctcataaggagtgtacaacccagatccctcacatgtgcagttcatgatagggtttgtgctcctatgagaatctaatgctactgatgatctgacaggaagcagagctcaggcagtaatgcgagtgatggggagaggctgtaaatacagatgaagcttcacttgctcgcctgccacttacctcctactgtgtggcccagctcccaacaggccccagacaggtaccggtctgctcctgggtgctggggactcctgcattaccacattcatttgtttttgccaattttcataatatctaccatatcttataacttttatttgtggaaatactgtctgtggctcaggatctaactaatgagtctcttctttcttaaataattccttgataaatttctaccagtcagtttaggctaacatacaaccccaacattcagtggcttacaaccacaaaagcttattttttatattttatatccactgtgggtcagctatggctctgctccttgtcgtctggatgacaaaacagcctatatctggcaccatgctagtcttacagaaaagctttaaaagaaatatggcaaaggcaaactaatttttaacgtttaacttgaaagttatgtatgtacttcacttcacaagtcactgaccaaagcaattcacatgactcattctgaggtcaaaaatatgaatgaataagtcacatgtccaaggatgacatcaacagaattgagaagtatgctccacatcaagggaaggatagcaaatattttaacaataatacaacccataggactgatcactgtaatggagatgagaacaatagaaactttaaaaatgttcttcctgtattcttatcaactcgttaatgtttcctaaaataatatatactttattggatatgagtctttattcattctgatcatttctcttctttatagtcccttcccagttcttttttcaaagatctacccatttctcagagttttaatcacattcccatgctgccttagacacttccttgatgtactcaacttaagtttgatccatctgttctttgaacaagtgaatattgattttgtagcactaaattttacaccacaaaaattttgcaagtgtgtcttcctgtattttctcctaagttaaacatacgataacatacttgacaaaagtggagccttgatgtatttttctttactatatagaagaaactttttcagagtttcatggttacagtactgctgctgccactacagctgggctctccaaaggcattttctgttcaggtgataatgtgaacaaacacatcatttgttaaagtgggaattgcttttgattttgtattcacctctcctagtgtgtacttggtggatatagaaatccataccagcccctgctcctatcacatgtttactggatgtcttaatggataatctttgggttgagagaaggaatattgacctctggt

SEQ ID NO: 11 - DNA, 1800 bp, Homo sapiens
gataactatcataaaaaattaacttacataaagtgtattaaattctcagtgggcacggtggctcatgcctgtgatcctaacactttgggaggctgaggcgggtggatcacttgaggttagaagttcgccctggccaatatggtgaaaccccgactctactaaaaatacaaaaaaaaattagccgggtgtggtggtgggcacctgtaatcccagctactcgggaggctgaggcacaagaatcgcttgaacctgggaggcggaggttgcagtgagaaaagatcgtgccactgcactccagcctgggcaacaaagcaagactccatctcaaaaaaaaaaaagaaagaaaagaaaagaaaagaaaaactcatatactgctatctgtgtcattagctgcagagtagacaacaggtataaaaatattgaaatagcctaggaatctaagaaaaagggaaaaaactcagtcacatatttaaaaaatataaatgcacacatacacatgaaaatatctcatatatggtaataccaaagtttgttaaatggaggtaaaagcaaagtattaatgaaattaataagccaggtgcagtggctcatgcctgtaatcccagcactttctgaggccaaagtgagacgatcccttgaacccaggagtttgagaccagcctgggcaaaacggtaaaaccaaaccctgtctccacaaaaaatagaaaagttagttgggcatggtggtgcacacctgtagtcccacctacttgggtggctgaggtggaaggattgcttgagccagggaggtagaagttgcagttagctgagatcgcaccactgcactccagcctgggtaagagagtgcaatcctgtctcaaaaaaaaaaaaaaataataaataaataaacaaaaacaagttaaaagaaactacaatccaaactgacaaatctagccaaaatcttaaaaaatatatcatgaattacagaaaatctagaatatagtcatattaaaaatggaaaagtgaaatgaacacattttataaaagaaaagacagcctataaagcgcctaacattatattaaaaggtccccacttttaggagacacaaataaaatgaaaataaagggaaggaacaaggtagccaaatagtatctataaaatgaaagtgaaaaagtcaattatccctttctctctcaaggtatcagaattgcagggtaaatacctaaagaagttaaattacaaaaaacttcatattctaagaatgaagttaacagcaaaatttattactatgtaaatttaaaaggctagcaaccacccataaatacagttttaacatatttttttcaaacagcaataaaataattcaacataagaaaacatttaaaaaatagaaacagaagttccaacacaatatatgaaggggctaagagaaacctacacacacaaactcagccctcaaaagaaccctaaaatctaatttagaagaattttttttttttttgagacggagtctcactctgtcgtctaggctggagtgcagtggcgcgatctcggctcactgcaagctccgcctcccgggttcacaccattttcctgcctcagcctccccagcatctgggactacaggcttccgccaccacgcccagctaattttttgtatttttagtagagactgggtttcattgtgttagccaggatggtctcgatctcctgacctcatgatccgcccacctcggcctcccaaagtgctgggattacaggcgtgagcgaccgtgaccggccactatcttctcgcttaatagcataattagaagataaaa

SEQ ID NO: 12 - DNA, 1800 bp, Homo sapiens
tcttaggcaagggacttaacttctctacctctctgttttctcatttgcaaaatgggaataatgataatactttattttatagattctaagactttttttcacattttaatatccctgaactcaggacatactttagtcagttaggttttgtgaatcattgtggatgtgacttgatactaccttaaattattagttctctccttgagggtgttttttttgttttgttttgtttttgattttggtgccattcgtatgaattcagactgcagttgagtacctgtgcatacattctcaaagaagtttgatttctttcctcctcctggtagcatgtacattttgtttctctctttttctccatggttggtttatttcatgtttaccttaacagggaggacccactggtggcctagtattatgtaggagttagattcctcatcttgggtgtcttttttctttcttaatagcacagaaaataatggtgcaatcttacaatccgtgatgttttatatctgatgtaatcaaacctaccttcaagggttgttgtcagcatatcttagaataatatgctaacgctcttagcctagtgcctggaatatagtaagcagtcagtttatgttcaggattgttttatatccttgttgtacttgttctgctttgctctacctttttctacttgttttacttgtttgtgtttatctcttctgctctgttgtgagctactcatgaataggtttggatttcttccaatatcttcagagcctaatggcacttggtaggattgctaaaacacgaactgagttgcataaaagaaaatgaatgctagtctgatttctattttagctgggcttatgaccttaagaaagtcactttacccctcaaggtatcagtttcttcacctttaaaaagagcagaatgaactattaaccttaaaatggctctcacctctaaaatttaattttagataaacttttaagttcagatttacagaaaagttgcaatgatagcacagaattctctttacccagtttctcctgttaacatcctgtattaccttagtttgtttgtcaaaactaatgtcctgttatctgttccagggtctagtctagaatactgcattggatttagtaaaatttgactttaagctaaaaatgctatactattttgtgtacctttagagttttgagtattatagaatattggttattaaaactatatgcatatgatggaattcacttaatttgtttcaatgttaggtttccatggcacatggaatacaaaatttgattaatgccatcagaatgattcacggtgtgtcaaggtattataatacctcagagagaatgacctcattgtttatcaaggtaagtttctgtgggaggaaattacatgtcatcctgtgaaaaagttagcaaatctttccttctgagccctaggaagggaatttggaagcctaagtgcctctcagagttttaatacttccagtggacaagaagagaaaggcgttgactggactcagttgtacttaattgaagggagtaattggtacttttctgtttatcagatgtaagccccaatacttaaagaaaatgtgcctggtggttttttaatttattcatttcaaatagttttgattctacccaagagataggagtgtgtctactattaacagtgcttctttagctactcccatgaacactatagtttgtgtaaaattttcctgtgtagcgccgcatggttaattatttaaaataattttttaaaaatgattaaacaatatatatttaaatttgctagccaatg

SEQ ID NO: 13 - DNA, 1800 bp, Homo sapiens
taggtatatcttcaaatatgtattaaattttaatgtaagtcactaagataatgaatatggattgtaaaatgtctaaacagtagagtaaacaagattgacccagaatcaatgaggcctcctgaaaatgtgatgtcgtcccttgcaatataattccttcccttattctcccattaacagaacctctcttttcagtcatgatgactcaattcccaagcaacagtaactgttctaacgtgatcaaaaccaagttcatctgaatcctttcctgcaattttgttttactgaggaaaaaatgaaaagaactctttcatcccacaggatttagaattataagaatatgagcctaaagccatcactattcaagtaggagctgcaagaataagaagcctgagaaaatgatgacaataaagagaggaagataagcttggcgacaggaaggtggagagattactctgatgctatttaagccctgatattctgtcgacacggagggtcactttcctctcattgttctgtgtgtttcttgcttcagccttcacataaacacatatacatttttttcttatattattttgagtcgtgcatctaccacttgggatagagaatcctgaccaaaacctcaagagaacaacttaggtaacaaaattacatggtaaacaaataacctaaaatgttttaccatgtttgtttcattatgtcaaaataattttaaaaaataataagcaatttaaaaatacataaaaagtaatcaaaataaatatggaggtataaatttttctatgacagagataacattggattgaatttaaaaagttaaacagaaaaaagaaaaaagaaaaaaggaaaaaagaaaccagaataagaacaaatagacaacccaagttactaaatggtatttttaagagacacatcccaaacaaaatgatacactgtttaaaaatgtaggatggaaaaaaaatatctagtaaaatttattttaaaaatggctgtggcaacagtgatataattaaaaaatagaattcaatgtgaagatcaattatttgggtaaagaggcctattttattatgacacaaagtcccttttactaggaacttataaaacagttaggagtctttattctcctagctgtaacagtttaacatatgctaaagcaaacccagtgaactatataaggactagtaggtaaagacataaccataataggggatgttaacacttcctctcagaaatcatcaggtcaagccaatataactataatgggaacaaacatgactcatgggatgtagtcataaagtgattctagttttgcttctcattgtcttttacagttgatgagtggagatatttcttccccttttccctgttcaggtaaactgggtagcctcacgatttattataaattaataattcctgttcattcatttactagagtctcaattcagcccctaaaatgtccagtcctatgtcatcatcaaagaatctaaccttgatttcaggcatatccccctttcccttgtaggtcctactgtggacaaacaacaaaagaaaggaagctgattggcttcccctctttttcctcccctaattcttttactccttctctccttctagctaatcaggtcctctaattagctggtgcggttggatgaagggattgggagagagatgatgctgcctctctatgaaggtctggtcaatggaaattacaagtctttttcccaataaactgtggaagaacagcttcccaccaaacttcaggtcatgattttgctgccacaaaaccactacagcctgctgcat

SEQ ID NO: 14 - DNA, 1800 bp, Homo sapiens
cgaagtatgtatttttcagtttaagcacaggacagttagttttatgaagtattttgaaaatgcatgaacttaatgatttaatattaaaagcttatggagatatcctaaacggcaacttaaattactcttccagataaaacatatatggtacttacatatactctttattaaataaacaaaaacaatccaacaactgcttgtcacctttttaagtatatttttatacttacagcatatatgtaaacaatatatttttctttcaaaagttagttattacaggatgcattaacaagatgccctctgggaaaatttcagctttcttttaatgaaacttatttcaaggttaagttaaaagaagtcacactaaagggaaatctgttgtcttcttcgggaaaatgatataagaatattaataatctaatagaataaatatgctttcaataatgaaacaaaaaaacacaacttcgcagctggctatttttattgtcctttgaagaattctctcccaagctactgaaacttaaagtatcacactgtgacagactttaatcccatcttacctggtgtccacatttctaaagaagctgcttattgcctcatcataaattcctttctaaaaagaaaaacatatgttaatagcatttaggttttgcttctcgcccaaatcaagttctttcaattttaattacataatactgtaatggctaggatgagactctgataagaaaaaccaagtatcacaaaatgcggagcctgactcttcccaatctccttagaagagtaaatgccagatctacagataaaaagttcagcagggggtgctaaagaaaggatttcacccataggactgtaggacaggtccatcaaatgagcgttctctggattcttcgtaatctttttaaaagctgactggttaaattaaggaatacaataaccacacgtgttatctattttacaaagtactttcccatatacaaatctcactgttctgcacacgaacaccaataaagaggagaccctgagaatcaggaaaaacgttaagactaaggtcgctggccagtgttcttttagtttagtgtgtgtctgacgcaacatgtttaaaaaccagtacgttctttactgggaagatcccatgcatcttaaacgggtcagatgaaagctcaagtcgattatttcctgcgtatgggctggaagacctagctgacaggcgagggggctcgcggcggcgggcgggtacctggttgaccgcggcgtgctcccttagtgccaggtcccagaagcagcagccgatctggtttccgcactggccgactgcgaccggaggagaggaaggaaagagaataggacattaagcaggaaacatggccgctggattccgcggcgaccaggtctctacccgcccggcgtagcttaccctgtacgaccaccgactgggtcatggtggtgcgccgccggctccgggagcttgctagcccgcggccgcttctgcattcccccagcaacggcgctctgctagagagctgccgtaagtcacgctactgccgcaagcctctgccgtcgctaagcacgtctgtaggttcaggctaaggttttccgtcagaaggccgcgcaagtgcacttgcgtgtcaccgttaccgtagcgactgggcttctggactgtatatcctagctgccttgtcaacatcttcgagcatcggcagctccggaggccggggtaactggcaggtaactaggctttggagtggtattttaaaatatgtgtcgttaagctgttcgtgggctatcagaggtttaattttt

SEQ ID NO: 15 - DNA, 1800 bp, Homo sapiens
gactctggtatagcatcgcatggcagatagcaggcctggaaggaaatcaaggtattttacccccaaatatatttctttgacatattttgaaatggtcctgcaaagctgtctcctgtggatgggggaggggccctggaatttgcattctgtagagaatccccttcccttactagtttttttttttccggagagcctgacaccttttaaggtccaataaaaaacactgtctattctctctgaagcctgctacctggaggcttcatttataagaaccttagcttcacaatctcccttaactcaagcatttctttgtgctgactttaaactcttcaggcaaaactcaactctttcaaccaattgccaatcaggaagtctttgaatctacctataacctggaagcccctctctcccagatgtcgcacctttccaggtcgaaccaatgtgtaccttacatgtattgatttatgtctttgcctgcaacttctgtccccctaaaatgtaagaaaaccaagctgtaacccaaccactttgagcacatgttctcaggacctcctgaggatctgtggctgggcagagacacctaaatgtgatctgcagaggattgcatagagaaagctctgcctgtggctgacatactgagaactaacctgggcgccttacagcaaaaccatccatgaagcagatgtggtggggctttcctgatcttttagggcccagatgcatttcactgagatgcatgttgcctttttctgaaagcgtctcataattagtctttacatgtgtttcaatctggtttcacactcgttaactgcttaaattgtagaaagcgcattttatttcagtccactagggggaaccagagggattaggcattggagctaacaagcctttaacattcttaatactgtaattagagaaatttctaggggttctcaaagtgagctgactttgagagagctgaataggggggtgttttttggtttcatttttgtgttttgtgtgtgtgtgtggggggttgttattgttgttttctttttaacttatttgcataaaagactattttttagagcaattttaggttcacagcaacattgaacagaaggggagtttagaaatatgtagatttccagcccttgcccccaatccacaaaatcagaagctttggggatgtggcccagaaatctctattgtaaaggttggtggaatatctgcctttggcaaaaatctacttaattatataaactttaagcccaccaggggcagggacacactgcaagttcctagcatccacagcaagatgtacaaacagaggctcgcaaggttttaaacatttttttttaaagatagaggtgaaatacttggagattgtcaatatttggagtatgagtcatatatttttccatgtcaatatttggaatatgagtcatatatttttccagttgaacaacttcaactgttaagcagaatccacataatttacttagagcccaaggaaaatctgaaaaccaccatttgaggaagacttccggctccagaaatgaaattttagggagcccccaaatcagaaaacagtatattgtttctgaaataagcatttaatgaaagactgacagattcctgtagatcttatccagactcagggaatagaaataaatgccctagattcaattgccattagacacagatgtttccatagtatcaaatacattctggcacactggccaacagacacaagttggagttacagggagaagagaggccatagagattgtcacaggggaggatttaatacattctatc

SEQ ID NO: 16 - DNA, 1800 bp, Homo sapiens
aaaaatatgtaacttaatattgaaacatagagataactcaaaatatgcctcaagcctcagagctcttcttttgtaataaccaataatttataggccatcttaaagaagaaatagaagctatcagctatgtatggacttcaccgttgtttttttgtgtttgtagacaagctatcttaggtcactgctttagcaaattaaattagctcagcattcactcaggagatgggtgaggaggaaataaaacaatggagatacagatagattggccaggaagctttagggtgtggggtagttggatgcagaggctatttagaaaccaggcttcagaataatggaaagaattgaataaatcctatgactctactaatgcaccaaagttttgttttgttttgttttttagtacagtgatgaaatgttaggtgatttttttaggtcaaacataatatgtaggcacagcagccaggctggagatgaaagtttgttcgtgagggattcaacctcaagagctctgtcaagtgcaggtaatagaggaattgaagatttactttagagtttcaggattaaatgaggtccagagctaaatctgggtagatttggaagagcagtcagatatgtattattctgagcaggactgtcattaacattttagttcaattaatttggaaattgagtagaaattaggaaattaggaggaatagctgaatgtagtttggagatgatcatgagttgagttttagatctgttcagttttgtatgattatacgtaattcaagaggagaattccaaaatgcatttggaaatatacaaatggagctggagagaaggagttagaggcaaatatacttattcatatttgttctcatagaggagacacttcactataaatctggatgagattttaaagtagagatttcagagaaaaggtcagatggctgagaactgagtgactcagcgatcactggaggccagccagtttggtgggatgggagtgtgggttcagcttgtgtgcttgggagacaatcccaaggaaagttcagatttttctccagaggtaatatcagatagacagtaagtcagagtaaggccatactgataaaatagcctagaggagaggtcacagtgcaaagaaaagaggacgattatctggctgagaggaaacaagaaatgtgagggggagacagactggagataatgatatcatgaggcagggaaagaatgttttaaaaagaggaagagaactatatcaaggtgcaccagatattgatgacaggttcagtaaaatgagcactgaaatatttctgttggttgtgacaacatggacatcatttagtgaccttattaagagttgttttcatggagaagccacagtgatgtgggttgtagaatgtgtaaacagttctagaaaggccaaaattcagggcagaagtgcaagtccagaagggttgttttcttggtgtgtattattttgttttaagataggaacaatttcagtatgtttacattcagctggaaaagatccaggagagaaggaacatttgaagatatgagaaggcaataatgataccttcttgacaaaccagcatatagtgtatttagactttagatctatagtttaatacactgaactaaatgaatgcattgacattgtcaaagcaagagaattgagcaaccagagaagtatttaccgaattgttaacctcgatggctctgctctgtaagggttatgtggttgactaattttagaagacttctcagtaacctctattgaatgatttggtacaaggacacgtaacctttggt

SEQ ID NO: 17 - DNA, 1800 bp, Homo sapiens
cagagcatcttttttcagacagtgatgacattgattctgtatatgataaagtgattctgcttctctttgacaacttgcatctctcctacatggaagtaagttttattcctgtcaatgttgtctttgtgtgtgacagattaggattaaattatggtttgacttttcctagcagcgtgatcatgggcaagtggcttttttttttttttttttttgagacagagtctcactctgctgcccaggctggagtgcagtggcacagtcttggctcactgcaactcctgcctcccggtccaagtgattctcgtgctgcagcttctcaagtagctggcatcaccaccacacctggctaatttttgtatttttagtaacgacgaggtttcaccatgttggcaaagctggtctcaaattcctggcctcaagtgatctgcccacttcagcctcccaaagtgttgggattacaggcgtgagccactgcgcccagcttttttaaacttttagattcatttaataggtaaattgcatgtcacgggtttgtagcttattctttcagaaactcttgcattatctgtagacgtggacgtaaatatccacctcatagggttttcataaaaaataattgagataatgtatgtaatgtttcacagtgctttgcagactatctaataaatagtagctattagtacaaacttgttgctttttggctagttctgcagtcacttttggaatcagtttatgaaccacacaaaaagtttgtcattattctgtagtcgctgtttttgatactgatgaaaatttacagtatccacctggattgtaccatctggctcacaagatttgatcttgaattgttagcacaatgaactacagcctcaaaggatgaaagtttaccttgcgggtattaaaaatgctagaagcttagaaagctgtttccaaagagggattccttgttaccaaccatgatagcattgctgtggtgtgtgtggtctcccagagtggctgctttgggattggaggtcacttagctattagaaggataggtctctttatattataccttgtaaacaaggctcatttggacttagggcttcctttagaggtgacacttaaccttggcaaggggatcatttctaccagtgggatccatctagcagatcagttcttaaattcggcttcatcatgtaaactgctacacagtgcaggctgactttgcattcaccattcagataccctctcctagttttatgttcttggcagcagtgaaaaggacctcagagttctcaggcaagatgatcttcactgagctatatgttggaggagcccagaaccatcatagttttagggtgtagttgaaaatccttactgtgaatggggtttatggcactgaaagttaccccttccctgggaattttgtaggaacccttgccacacagatcaatggagtggtgggaagaagcaccacaagacaggccttttttttttcctttaaaaaatgtttgaaagctcctactaatatggttaggaaaacttctgccacatctgaccataaaatgagagttgctcaaaatactagtgcagattactctgacatgtgaaatacaagacttcaggattgatgctagaaaaatacagtgaacttgaaaagcttatccctaaactgtaatcctcccaaacaaaacaaaaacaaagcaagaaaaaaagccttatttaaaattgtcaaaagaggctgaattttatgaagttaccaaaagtatatctttagacttttcttctgatttggtattttatgtatttaagcagtta

SEQ ID NO: 18 - DNA, 1800 bp, Homo sapiens
agggcgcgggccaggcgagttatagaggaaggttgttttgctttgtttttgttttctaatctggtggagaagagacaaaaggagaaatgggaaggaaaacaggggcctctctggcaagcaagtcgcaggtgtgaggcccagaagcctgaacagtaatagcgcattggaggcacctgagaggctcagtgacggcgtcttcacccagaggcactattcaaatagcaactcgtgtggcatgggcaccggccactcagaacgggcagcggcagggacagcgggatggcaggaagtcctgaccatccgacctgccctgatgtgtcatgatgtttccttgtgcatgtgcatttttcatgaggcgtgtggccatctcttatcagcctttgtgacctcatttcctcctttttcaaatgagcacttccaggagcccaggggaagggttggctttggagggtttggccagggttggggggcacctccaggcccctgcggagtggacagcgcagttgtgccttccagctgctgcttaggtcttagggaaactcatctgctcaagggggcttagggaatctgatttgcttttccttttctcagcctttctgagcatagggagaaggaggaagcagttcgaatcccattgcgggctgcagagagttctggatgagtaagcaccaaaaacgtgggaagtttctgaaaagggtttctcagccagcagtgctttcaccccttgctgggcgagtcgctgccgtgcagtgcagatgactcatctgggtaactcccttgacagaaggagccagccaagctgcagcaaatgtgcgttggggggatggggaagaaaggaatgaacacagagcattccctgtcaccagacgctatgctagacactatgtgtatcatcttattaaatgatcccaaataccctgtgacgattccatcccagatactctggccccaccgcacaccttgggatccactacatgaacacttcctttcctgacgtccccagggcccacctatttcacatgaagctctggatactgtcagaaaaaaacatccaacacctggagataaggccaagaaggtggccggcacatcctttcaatatcgtaggtggtaacaggccacctttatgaatctcgcagtctaatacaatagccactagccaagtgtggttacagagctcctgacatacgccagtgtgaattgagatgtgctgtaagtgtatgatctacactgggtttcaaagacttagtatgacaaaaattataatgtaccttattaacatttttaatgttgagttcatgttgaaatagtatttttgatatattgggttaaagaaaacatcatttaaattaatttcaggttaggtgaggtggctcatgcctgtaatcctagcactttgggaggccaaggcaggaggattgcttgagcccaggagtttgagaccagccttggcaacatagagagaccctgtctctacaaacaaaaaacaaaacaaatagccaggtgtggtggtggagatattttgcattctttttttatagtaagtcttcaaaatctagcacatctcagtttggaccagccacatttcaacttcttaagagtcacatacagtgagtagctaccatattgagcagtgcaggtttcgatcctatgcttagaaggatcatgaccaacaaataaaggcagttcaagttctctagtgttccagtgtgagctggaaggcagttgtctctacaaagggatctttttctatccaaggcaaccacagggctggggatggtaggtgtggagctgtacctt

SEQ ID NO: 19 - DNA, 1800 bp, Homo sapiens
ctgacaaaaaattaatcacaacaaaacgttatataaaacgtgtcttctcaaaatgaacacattgtttccagctgttaaacatcaacctgtctcaaaggaactgaggtaaaagaagcattttcatcccacctgttcatcttactctgtttcacaaaatggaggccacaaacaccatttcatacaagcatatgatattagcaagaaaaacatctcagctccaaaatgcacagtacaaggtgataacaccatcatcaacgagatgttaccatcgagtgaagtaatgtttaccaaagacaaacacagcaggaatttacatccagttgcccttgacatagattttcctcttggtccacatccttaaattcgggttgtagtctttttgcaacctccacagtggttctggaaccatctagtttttaattccccacggacacttttcaattattttcccagcacacttgcagcatgtggctcttcctgcagagctcagctgaaggctcctggtgattggatgtcaagggccttttggtctcttccccagcaagatcgaggaattgctttcggacccaaactaaattagtagtcctatcgtatatgagacagagacaactgactgctttacacccgcaggttgagaaggttcatagaacataaagaaccagagctttggcagagctacatgcctgaatggatgcacattctcaggtgctacaattgcctgtcactgtcatactggagcatattagattgttaagtcatgactaacagacagaagtatgttatccacctatatatactttgcttataaaattgtattatttggtttcagggcagcctatagtgaaacgttcatttgctccactgttgagagatgattcttatttctctcaaattatacttaaatatggctcaatcaaaatctgcacataaagccatgcaggcacattatataactttttgggcatctgagttttctaagacaattatatatttttcagaaagcaaattctatattctattcattaaagtattaatatgaacagagaactctcttctatctcaaacacctaatgagaacaaaacttccccaaatggcctttgcctagcagccagtcttcctcctgtgtgattactcagatcttttaatctttcaaagctctaatccatgacatctctccccagtacactagctcccagtggaagttctcttcccaaagaagttatatactgtctgcaatgtgtcttaaatgtagttaacttcaggcatctgtgggtgttatattcttcaataaatttcaaagtcttttaacagaagggatcatttttatcttcccagtgcctgccagaatgtaagatacatactaagtactcaaaaaactgttgattaggaaagtaaataagtaatcaatatagtgacaacagaacattatttatggcactcacaaagcattttccctatatatgatctcatttgatacaacctgtcagttaagtaaactggattttaaaaataacttctattttacaaagaataaaacagggtttgggaaatttctcccagtgacacacacacacacacacacacacacacacacacacacacaccccaaagttcttagagaaatacaagtgaagctgtgcttgataggcagtgacctactggatcatacacattctctattgttttctttacattgtatcccacatgtcacccacaatgctctgcttaattgaaagacctggtataacaggcttaaagtcacacaagctacgatgggcacccaaattttctgttcttcc

SEQ ID NO: 20 - DNA, 1800 bp, Homo sapiens
tgcagtgtcccctctcaaaactaagaaagctccaggtaacttactttgcctgcaccgccagcctggacaccccacccggggcaacccctacaaatctgagatgacacaaactgtcatttcaggcttagcccaaacattccgtctttcaggagcctccccaactctactcctgcccccggagaagtaagtgctccctcctcagcattcccgtaagcaggtgtgtgccttaagtatatacatactcatcacactgtactgcagctgcctgtgctcctgtgtcagaatcctacttctgactgagagcactccctccctgaggacagggaccagctgtgttcatctgagtacccacatctctgtccctccatccccaaagcacagtgcctggctggcacacagcaggcgctccataaatgactattatatgcaggggagggtaggtgggtgggtgggtgggtggatggatggatggatggatggatgggtggatggatggatggatggatggatgggtggatggatgggtagatggatggatgtgaatggaaggatagatagcagcaggaggtgggtaagatctggggccaagaaagagatgttctggatgagccatgctacaacactgacaacagactggtggtgctctaccagcagaaggcctaaaaaagaggatacactcattaaaatcttttcaaattcagtattccatgaacaagcacctagggaactgtgtgcactgccaaagctgtttacctaagcataattttgttgccattatgcgacgcgttgtctagtggttataacagaagacgagttactgtgacgagcctggaaagccatttcttgttgccttatcgttacccaggtctccctctctgtgacgaattcatccactcatttaacaaatatttgccaattgctcatcaagtcccaaccaccactctgggtgttgcagacacaactcacgttactccctcactatgggaagccctataggaactaaaggaagttctcactgaacaagtacttcttgaggggccaagtcatccaaaacagggttgaaacgcaaacccagaggttgcaacaccatttccctccctgccttcttaagcagaatgtactgaatgtgacagctaagttgggatccccgttctgaaatgcaataatgctccatctccttccagacttgtatggtgagctgttcccaaacacagtagactccagagagctcctacctttgagttcttgcatcaatgttttctagtttttctgccaacacattaacttttcccaaagaccaacaactggtgtttttgagtcagtctatgtctctgtagcagctccttctgatgacatgagctaaaaataacccagctcattaaaatcatgtgtaaaataagagtacatagttgccagtgaaagactggggcaggcaggcagccagcaggacaagggatggacattgggatatctttgctggccagcctgagattctcattttgcttattcactggttcctagctgtgctcctgataagggagactgctggacagctgatgtcttaactgccccccaaaataaatgaactatcctgaccttcttaggtaagatgctgctgacaatgtctgactgcaacctgacccaagtgcaacattaacaggtcagatctggtcccggaagaacaaagaagatagaaaccaacccatcaaatctcattagtctcaatagcataagatgccagccatcccaggcaggtttgaaaggaggaaataagctgtatgtgagcgtgccagtcagatg

SEQ ID NO: 21 - DNA, 1800 bp, Homo sapiens
ttttttacaaacacttgaagggaaaagtctgtttgcactgactgagctctgaatcaggtcaaataaagacaagctttgcgagtggggttttctagggaactgtaagacaggtcaagtaatggagattctctgagaatggggcttggaaagagctccaaccccattttagcctctcctgtggcttctaggctactggttttcactgtggttgtggggctgttggtttttaaggctacagcagaactggaaagaaggcagagggattaggacaagttaaaacactataaaactcactatcattaccaagacacagtcatttttcttgcataaatgcttcttgaattattgcaagcctttgattaattttcagcattctgaaacagtggatttagagttttccagtgttctcattggttttatagaaaaaataattttcagaagaacttattctgcatttctcatgatgtcacctgatgccttgtttgaacttttgtaattaaaaaaatgttttcttcatattgctaagtattttttttgaaaaaaattgcaatgaatatgtagaaactttgttttccattttagatgactcttttctggtaaataaaatgaatgcttctcattctcattctcaaaatatgagtcataaatattttgaaacggctaatgtttgtgaaatatatttaataatggattatgtagtcatgcagtgaaaacaaggcgataattcagaactcagtaatatgggatacaggcatatggttaggtacggaccctcaaggttcagaaagcctgggtcataccatgatcttccctctgctactgttaggatcttggcagattactcatcctctgtgctttagtttccttagctataaaatggggcaaagatggtacctatttaatgaagtgattggaaaatttaatgggttaatgcttgtaaagtccaaagcaaaatgtccagaatgaaatgcttaaaagatattagttattattaaaaagctagagatttttactctaaaatttaatgttgacaatctaatgtgggttgtcacttccatagccatctaactggtctttctgttttatttccttgctttgaatggtcctatacataacagctggattctaataataactaatataggaccattcttaggtgaagaatctaactgttatgtataggaccagtcaaagcaaggtgatgggttattgcatcattcagctgcctgaaaaaaaaacttggtgttctttatcaccacattgctcttaataccaatctctggtcttctgcaacgagacctaacccatgtcttttttttttttttttgcctgtggaccagattggtgctatactccagccaaaccaagctatctctgttcctgaagaagttctatgcttcccttcctctttgtctttcctcttattacctctttttcctggaattattttctatcctttttacttgaaatcctattcttctctctactgaaattctgtttcctctatcctcccttttatctgcttcaccaagaatgaatatttcacatgtctatagcatatggcacttaatttctcttctataatatttctttgtactcttgttactggccactgaaccataacttttggaagacatactctacatcctactaatctttgactatcctgcaggaactagttcagtgcttgtactcaaatgatgctaatgatgcttaaaaacgtatttcagaatgaaaactaaaattctgaggaaacaaacacctgccctttgaaattcttccccttaagtggagatgtta

SEQ ID NO: 22 - DNA, 1800 bp, Homo sapiens
caactaatgaattagaatgttggttttaaatttgtttgctctcaataattattagctgctttaaatatgaattcatgccttgataacaaatatgttccaaggaagtaccgactgaaagttcagtaggcttcctgtcttcaaagtgattatgataccagtggatgctgataaaggcagactttaattcaaagagataaatagagttgacatgaaagtttaaattactgcaagcgaccttttcattttctttcccttttctgtcatttatccatatttgatcatttgtatttttagctcctttatgcctgcctgttacggcattatctcattatcgagagaattttgaattaccttttaggtttgaaatagtagcatgattaggcatcatttagattgacgacagttgttgcatgcatgaatgcctttactatgtctttattccagacagctcttggtaatgttgatataacattttcttattttccccttgaattttaatttgaaaactatttcttagttattcagccagttacttatttaaaaacttgctttgagactgggcacggtggctcatgcctgtaatcccagcactttgggaggccgaggcgggtggatcacctgaaatcaggagttcgagaccagcctggccaacatggtgaaaccctgtctctattaataacacaaaaattagccaggcgtggtggtgcacccctgtaatcccagctactcgggaggctgaggcaagagaatcacttgaacctgggaggcagaggttgcagtgagccgagattgcgccattgcactacagcctgggtgacaagagcgaaactctgtctccaaaaataaaaataaaaacttgctttgaatctaaaaatggtatcatttagtgacactacctttaaggagatctaaatcttatgatggcgatagatggaacataaattattataatcctaagtggtaggaactgagataagatcatttgatagaaaattttattgcaccctttttgataattacctttaaggtgctactacatctaagtgtaatattacatctgtttgggtctctgtgtgcattcatatatatatatgtgtatatatgtgtgtatatgtatatgacaagaataaaagctcttgggcatttttggggattagtagattaaaatttatttacaattaagatgactttggcttaatcaagagttaatcaagagcaggtttctgagtgccaaagtagcacctactttgtatttttgagtactcacctttggagagtgttattaacaaggcatgctagcttttgaaatgttgttatatggagatacagagagacagaaaatttgactgatcttgtgttcacaaactgaagcaaatatgaaatctagtatatgatttactaaacaaatataatggagctatgaatgactttaatctactgataatccatagagtttatttcaatctaggatgtgattccttgtttgcactacctaaacctttgcctcccacattttatgttgtggcagaaattattaataagcttgtcttctaatctctcccaggaaagtgcttttgtagcctttctgctgaggttaccgtcaaggtatcaaaggaagccttttctcagttaatagctgttgatattacattatatatatatcctataactaggaagtataatacttgtttggaagtaaatttttgaggtgcattcattttccttcaatgtttaaaaaacttagctatgttttaattgaaggatttttaaagcatatatctcattgttctatcatcttga

SEQ ID NO: 23 - DNA, 1800 bp, Homo sapiens
tggattagagctgttagtgaagtgcgtattatgtgttaagtacttcataatgacagccttacaaaaaaatggccctaatgcaaacggaaaaaaaaactgtgtgtgtttttgtatatatatgtgtgcgcacacacacaattcaagtttagtacttgtggtttttattttgtgaactgtgtaaaataaattccctaagactgggttaagggaaaaaatgtaaccatgctttttaccaaataatcaccttaatttggtacatttctctcttctttctagcttcattttaggatcttagattacacacttactggatgttatgcagatcagcattcagttcaagtttttgcatcaggaaaaccaaaaataagtgcacgtaagttacacagattcaaactaacttaagggaattaaaatggtgtttgcagttattacctaaggtaaaatttgaatgtaacagaacttagagattagagaataatcccaatttataaaattctgggctgattctaccccacataacagtttgtgtactttacactgccctctgccgccttcttgtttataaagtggtctggataagcaaagattgggatagaggggaacttcagtgtaaaattggatttgtttttctctggttattcagagatgtgattcacagagactgtgatgttggacagacagggcttaagcctggctcagccatttcattagctgtataactttgggcaagttagcgaacttcttgaagttttaattttcttgtgtataaaatggaaataatagtgcgatttacctcaggattgttatgaggtttagataagattatattgacttagtatgatgcttagtgtatagtatagatttagtagatactagagatgattcttatttaaaacttttaaatatctttagcaaatgttgcaatcaataagcattatttagcaccttctctattctatcaataggaagacaataaaaaagacgtttcctgattttagtatgtttaatggttggtaaagtatagatctatatgtgtgtacacacacacatacacacacacacacacacacacacacacacgaaacatatgaaaatatgagacaaaaaacaatttataaatgaatcaaaagtatttaattcagatcaaatagaaatatgtaacatcgaatagtagctaaaatgaggtagagttatctaaacttaaacaacctttttgcctcatacattaaggagaataaagctgttatatatgtggataagaataatcccaatttacacacatttttattattatagttagataaatatttcttaaactaaggattagggttcctaaaataaaaagaataggaaaggactctccatgtacatttcaagagaattagagaactcagaactacagaattctaagcaactatttgtgcttgtcaatataatttctactatgtaatgtaaccagaataaaccagtattctttacttccctaggcttgccctgtgttttctcttacatcatttttttgttgtatatatgagagactgaaagcaaacagaccagtattttagattgtctatgggaaaataatttatttatgttaactaaacatactgcagtaatttccagagaacataatacaagataggaacatacctgcttcttaatacaaaatatgttgaaagaactcattttaaattaatattatgacccatcttttctttcctctgttagtttcagaatgtgactgccagtttgtgcaattatgtaaaactttattttgccatcatcttgacaataaaatct

SEQ ID NO: 24 - DNA, 1733 bp, Artificial Sequence
  Description of Artificial Sequence Synthetic polynucleotide
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattg

SEQ ID NO: 25 - DNA, 798 bp, Artificial Sequence
  Description of Artificial Sequence Synthetic polynucleotide
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 26 - DNA, 522 bp, Artificial Sequence
  Description of Artificial Sequence Synthetic polynucleotide
tcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 27 - DNA, 2348 bp, Artificial Sequence
  Description of Artificial Sequence Synthetic polynucleotide
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccgaattcgcggccgctaaacccagctttcttgtacaaagtggcaactttattatacatagttgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 28 - protein, 429 aa, Homo sapiens
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 29 - DNA, 1290 bp, Artificial Sequence
  Description of Artificial Sequence Synthetic polynucleotide
atgcagctgagaaaccccgaactgcacctgggatgtgccctggctctgagatttctggccctggtgtcttgggacatccctggcgctagagccctggataatggcctggccagaacacctacaatgggctggctgcactgggagagattcatgtgcaacctggactgccaagaggaacccgacagctgcatcagcgagaagctgttcatggaaatggccgagctgatggtgtccgaaggctggaaggatgccggctacgagtacctgtgcatcgacgactgttggatggcccctcagagagactctgagggcagactgcaggccgatcctcagagatttccccacggcattagacagctggccaactacgtgcacagcaagggcctgaagctgggcatctatgccgacgtgggcaacaagacctgtgccggctttcctggcagcttcggctactacgatatcgacgcccagaccttcgccgattggggagtcgatctgctgaagttcgacggctgctactgcgacagcctggaaaatctggccgacggctacaagcacatgtcactggccctgaatcggaccggcagatccatcgtgtacagctgcgagtggcccctgtacatgtggcccttccagaagcctaactacaccgagatcagacagtactgcaaccactggcggaacttcgccgacatcgacgatagctggaagtccatcaagagcatcctggactggaccagcttcaatcaagagcggatcgtggacgtggcaggacctggcggatggaacgatcctgacatgctggtcatcggcaacttcggcctgagctggaaccagcaagtgacccagatggccctgtgggccattatggccgctcctctgttcatgagcaacgacctgagacacatcagccctcaggccaaggctctgctgcaggacaaggatgtgatcgctatcaaccaggatcctctgggcaagcagggctaccagctgagacagggcgacaatttcgaagtgtgggaaagacccctgagcggactggcttgggccgtcgccatgatcaacagacaagagatcggcggaccccggtcctacacaattgccgtggcttctctcggcaaaggcgtggcctgtaatcccgcctgctttatcacacagctgctgcccgtgaagagaaagctgggcttttacgagtggaccagcagactgcggagccacatcaatcctaccggcacagtgctgctgcagctggaaaacaccatgcagatgagcctgaaggacctgctgtaa

SEQ ID NO: 30 - DNA, 1290 bp, Artificial Sequence
  Description of Artificial Sequence Synthetic polynucleotide
atgcagctgcgcaaccccgagctgcacctgggctgcgccctggccctgcgcttcctggccctggtgagctgggacatccccggcgcccgcgccctggacaacggcctggcccgcacccccaccatgggctggctgcactgggagcgcttcatgtgcaacctggactgccaggaggagcccgacagctgcatcagcgagaagctgttcatggagatggccgagctgatggtgagcgagggctggaaggacgccggctacgagtacctgtgcatcgacgactgctggatggccccccagcgcgacagcgagggccgcctgcaggccgacccccagcgcttcccccacggcatccgccagctggccaactacgtgcacagcaagggcctgaagctgggcatctacgccgacgtgggcaacaagacctgcgccggcttccccggcagcttcggctactacgacatcgacgcccagaccttcgccgactggggcgtggacctgctgaagttcgacggctgctactgcgacagcctggagaacctggccgacggctacaagcacatgagcctggccctgaaccgcaccggccgcagcatcgtgtacagctgcgagtggcccctgtacatgtggcccttccagaagcccaactacaccgagatccgccagtactgcaaccactggcgcaacttcgccgacatcgacgacagctggaagagcatcaagagcatcctggactggaccagcttcaaccaggagcgcatcgtggacgtggccggccccggcggctggaacgaccccgacatgctggtgatcggcaacttcggcctgagctggaaccagcaggtgacccagatggccctgtgggccatcatggccgcccccctgttcatgagcaacgacctgcgccacatcagcccccaggccaaggccctgctgcaggacaaggacgtgatcgccatcaaccaggaccccctgggcaagcagggctaccagctgcgccagggcgacaacttcgaggtgtgggagcgccccctgagcggcctggcctgggccgtggccatgatcaaccgccaggagatcggcggcccccgcagctacaccatcgccgtggccagcctgggcaagggcgtggcctgcaaccccgcctgcttcatcacccagctgctgcccgtgaagcgcaagctgggcttctacgagtggaccagccgcctgcgcagccacatcaaccccaccggcaccgtgctgctgcagctggagaacaccatgcagatgagcctgaaggacctgctgtaa

SEQ ID NO: 31 - protein, 419 aa, Homo sapiens
MGWRAAGALLLALLLHGRLLALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 32 - DNA, 1260 bp, Artificial Sequence
  Description of Artificial Sequence Synthetic polynucleotide
atgggctggcgagctgctggtgcacttctgctggctctgctgcttcatggcagactgcttgctctggacaacggcctggcccgcacccccaccatgggctggctgcactgggagcgcttcatgtgcaacctggactgccaggaggagcccgacagctgcatcagcgagaagctgttcatggagatggccgagctgatggtgagcgagggctggaaggacgccggctacgagtacctgtgcatcgacgactgctggatggccccccagcgcgacagcgagggccgcctgcaggccgacccccagcgcttcccccacggcatccgccagctggccaactacgtgcacagcaagggcctgaagctgggcatctacgccgacgtgggcaacaagacctgcgccggcttccccggcagcttcggctactacgacatcgacgcccagaccttcgccgactggggcgtggacctgctgaagttcgacggctgctactgcgacagcctggagaacctggccgacggctacaagcacatgagcctggccctgaaccgcaccggccgcagcatcgtgtacagctgcgagtggcccctgtacatgtggcccttccagaagcccaactacaccgagatccgccagtactgcaaccactggcgcaacttcgccgacatcgacgacagctggaagagcatcaagagcatcctggactggaccagcttcaaccaggagcgcatcgtggacgtggccggccccggcggctggaacgaccccgacatgctggtgatcggcaacttcggcctgagctggaaccagcaggtgacccagatggccctgtgggccatcatggccgcccccctgttcatgagcaacgacctgcgccacatcagcccccaggccaaggccctgctgcaggacaaggacgtgatcgccatcaaccaggaccccctgggcaagcagggctaccagctgcgccagggcgacaacttcgaggtgtgggagcgccccctgagcggcctggcctgggccgtggccatgatcaaccgccaggagatcggcggcccccgcagctacaccatcgccgtggccagcctgggcaagggcgtggcctgcaaccccgcctgcttcatcacccagctgctgcccgtgaagcgcaagctgggcttctacgagtggaccagccgcctgcgcagccacatcaaccccaccggcaccgtgctgctgcagctggagaacaccatgcagatgagcctgaaggacctgctgtaa

SEQ ID NO: 33 - DNA, 4347 bp, Homo sapiens
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgggctggcgagctgctggtgcacttctgctggctctgctgcttcatggcagactgcttgctctggacaacggcctggcccgcacccccaccatgggctggctgcactgggagcgcttcatgtgcaacctggactgccaggaggagcccgacagctgcatcagcgagaagctgttcatggagatggccgagctgatggtgagcgagggctggaaggacgccggctacgagtacctgtgcatcgacgactgctggatggccccccagcgcgacagcgagggccgcctgcaggccgacccccagcgcttcccccacggcatccgccagctggccaactacgtgcacagcaagggcctgaagctgggcatctacgccgacgtgggcaacaagacctgcgccggcttccccggcagcttcggctactacgacatcgacgcccagaccttcgccgactggggcgtggacctgctgaagttcgacggctgctactgcgacagcctggagaacctggccgacggctacaagcacatgagcctggccctgaaccgcaccggccgcagcatcgtgtacagctgcgagtggcccctgtacatgtggcccttccagaagcccaactacaccgagatccgccagtactgcaaccactggcgcaacttcgccgacatcgacgacagctggaagagcatcaagagcatcctggactggaccagcttcaaccaggagcgcatcgtggacgtggccggccccggcggctggaacgaccccgacatgctggtgatcggcaacttcggcctgagctggaaccagcaggtgacccagatggccctgtgggccatcatggccgcccccctgttcatgagcaacgacctgcgccacatcagcccccaggccaaggccctgctgcaggacaaggacgtgatcgccatcaaccaggaccccctgggcaagcagggctaccagctgcgccagggcgacaacttcgaggtgtgggagcgccccctgagcggcctggcctgggccgtggccatgatcaaccgccaggagatcggcggcccccgcagctacaccatcgccgtggccagcctgggcaagggcgtggcctgcaaccccgcctgcttcatcacccagctgctgcccgtgaagcgcaagctgggcttctacgagtggaccagccgcctgcgcagccacatcaaccccaccggcaccgtgctgctgcagctggagaacaccatgcagatgagcctgaaggacctgctgtaacaactttattatacatagttgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggatccctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaa

SEQ ID NO: 34 - protein, 3 aa, Artificial Sequence
  Description of Artificial Sequence Synthetic peptide
RGD

SEQ ID NO: 35 - protein, 5 aa, Artificial Sequence
  Description of Artificial Sequence Synthetic peptide
RGDSP

SEQ ID NO: 36 - protein, 6 aa, Homo sapiens
LKRHQR

SEQ ID NO: 37 - protein, 6 aa, Artificial Sequence
  Description of Artificial Sequence Synthetic peptide
GRGDSP

SEQ ID NO: 38 - protein, 7 aa, Artificial Sequence
  Description of Artificial Sequence Synthetic peptide
GGRGDSP

SEQ ID NO: 39 - protein, 8 aa, Artificial Sequence
  Description of Artificial Sequence Synthetic peptide
GGGRGDSP

SEQ ID NO: 40 - protein, 4 aa, Artificial Sequence
  Description of Artificial Sequence Synthetic peptide
  SITE (1)..(4): This sequence may encompass 1-4 residues
GGGG